SEQUENCE LISTING (8 sequences)

SEQ ID NO: 1 - protein, 15 aa, Homo sapiens
HLDMLRHLYQGCQVV

SEQ ID NO: 2 - protein, 17 aa, Homo sapiens
RLRIVRGTQLFEDNYAL

SEQ ID NO: 3 - protein, 18 aa, Homo sapiens
TQRCEKCSKPCARVCYGL

SEQ ID NO: 4 - protein, 15 aa, Homo sapiens
GVGSPYVSRLLGICL

SEQ ID NO: 5 - protein, 15 aa, Homo sapiens
PAREIPDLLEKGERL

SEQ ID NO: 6 - protein, 15 aa, Homo sapiens
TLERPKTLSPGKNGV

SEQ ID NO: 7 - protein, 9 aa, Homo sapiens
KIFGSLAFL

SEQ ID NO: 8 - protein, 9 aa, Homo sapiens
RLLQETELV

(19) United States
(12) Patent Application Publication
(10) Pub. No.: US 2017/0261508 A1
(43) Pub. Date: Sep. 14, 2017

(54) METHODS FOR MONITORING CD4+ T-HELPER TYPE 1 RESPONSE IN CANCER AND IMMUNE RESTORATION

(71) Applicants: Brian J. Czerniecki, Tampa, FL (US); Gary K. Koski, Akron, OH (US); Jashodeep Datta, Philadelphia, PA (US)

(72) Inventors: Brian J. Czerniecki, Tampa, FL (US); Gary K. Koski, Akron, OH (US); Jashodeep Datta, Philadelphia, PA (US)

(73) Assignee: THE TRUSTEES OF THE UNIVERSITY OF PENNSYLVANIA, Philadelphia, PA (US)

(21) Appl. No.: 15/417,204

(22) Filed: Jan. 26, 2017

Related U.S. Application Data
(63) Continuation-in-part of application No. PCT/US16/20987, filed on Mar. 4, 2016.
Application No. PCT/US16/20987 is a continuation-in-part of application No. 14/985,303, filed on Dec. 30, 2015, now abandoned.
Application No. 14/985,303 is a continuation-in-part of application No. 14/658,095, filed on Mar. 13, 2015, now abandoned.
Application No. 14/658,095 is a continuation-in-part of application No. PCT/US16/20987, filed on Mar. 4, 2016.
Application No. PCT/US16/20987 is a continuation-in-part of application No. PCT/US15/20613, filed on Mar. 13, 2015.
Application No. PCT/US15/20613 is a continuation-in-part of application No. 14/985,303, filed on Dec. 30, 2015, now abandoned.
Application No. 14/985,303 is a continuation-in-part of application No. 14/658,095, filed on Mar. 13, 2015, now abandoned.
(60) Provisional application No. 61/953,726, filed on Mar. 14, 2014.
(60) Provisional application No. 61/953,726, filed on Mar. 14, 2014.
(60) Provisional application No. 61/953,726, filed on Mar. 14, 2014.

(51) Int. Cl. G01N 33/574; A61K 39/00; C07K 16/30

(52) U.S. Cl. CPC G01N 33/57415; C07K 16/3015; A61K 39/0011; G01N 2333/57; G01N 2333/70596; A61K 2039/5158; A61K 2039/57; C07K 2317/24

(57) ABSTRACT

A method for diagnosing or treating a mammalian subject having, or at risk of developing cancer, comprising: generating a circulating anti-cancer CD4⁺ Th1 response from antigen presenting cells or their precursors and CD4⁺ T-cells from a sample of said subject's blood which causes secretion of interferon-gamma (“IFN-γ”); and detecting said anti-cancer CD4⁺ Th1 response to determine if said response is depressed. A method for restoring HER2-specific CD4⁺ Th1 immune response in a HER2-positive breast cancer patient in need thereof, comprising: administering to said patient a therapeutically effective amount of a dendritic cell (“DC”) vaccine comprising autologous DCs pulsed with HER2-derived MHC class II binding peptides (“DC vaccination”) to elevate said patient's anti-HER2 CD4⁺ Th1 response, and measuring said anti-HER2 Th1 response of said patient pre- and post-DC vaccination to determine the amount of increase in said response.

This application is a continuation-in-part application of, and claims priority and benefit from, International Application No. PCT/US2016/20987, filed Mar. 4, 2016, which in turn is a continuation-in-part application of U.S. patent application Ser. No. 14/985,303, filed Dec. 30, 2015, which in turn is a continuation-in-part application of U.S. patent application Ser. No. 14/658,095, filed Mar. 13, 2015, which in turn claims priority and benefit from U.S. Provisional Patent Application Ser. No. 61/953,726, filed on Mar. 14, 2014, the entireties of each such application being incorporated herein by reference.

This application also is a continuation-in-part application of, and claims priority and benefit from, International Application No. PCT/US2016/20987, filed Mar. 4, 2016, which in turn is a continuation-in-part application of, and claims priority and benefit from, International Application No. PCT/US2015/20613, filed Mar. 13, 2015 under the Patent Cooperation Treaty, which in turn claims priority and benefit from U.S. Provisional Patent Application Ser. No. 61/953,726, filed on Mar. 14, 2014, the entireties of each such application being incorporated herein by reference.

This application is also a continuation-in-part application of U.S. patent application Ser. No. 14/985,303, filed Dec. 30, 2015, which in turn is a continuation-in-part application of U.S. patent application Ser. No. 14/658,095, filed Mar. 13, 2015, which in turn claims priority and benefit from U.S. Provisional Patent Application Ser. No. 61/953,726, filed on Mar. 14, 2014, the entireties of each such application being incorporated herein by reference.

### ACKNOWLEDGMENT

The present invention was developed in part with government support under grant number R01 CA096997 and P30-CA016520 awarded by the National Institutes of Health. The government has certain rights in this invention.

### FIELD

The present embodiments are directed to progressive loss of immune response in cancer, in particular the loss of anti-HER2/neu CD4⁺ T-helper type 1 (“Th1”) response in HER2-driven breast cancer and the restoration thereof, and diagnostic monitoring methods, treatment methods and tools based thereon.

### BACKGROUND

Breast cancer (“BC”) is a leading cause of cancer-related mortality worldwide. See, Jemal, A., et al., Global Cancer Statistics. CA: A Cancer Journal for Clinicians 61:69-90 (2011). Through the development of gene expression signatures, at least four broad phenotypes of breast neoplasms are now recognized: luminal A and B, basal-like, and human epidermal growth factor receptor-2/neu (“HER2^{pos}”). See, Perou, C. M., et al., Nature 406:747-52 (2000). HER2 overexpression, a molecular oncodriver in several tumor types including about 20-25% of BCs (Meric, F., et al., J. Am Coll. Surg. 194:488-501 (2002)), is associated with an aggressive clinical course, resistance to chemotherapy, and a poor overall prognosis in BC. See, Henson, E. S., Clin. Can. Res. 12:845-53 (2006) (“Henson, et al.”) and Wang, G. S., Mol. Med. Rep. 6(4):779-82 (2012) (“Wang, G. S., et al.”). In incipient BC, HER2 overexpression is associated with enhanced invasiveness (Roses, R. E., et al., Cancer Epidemiol. Biomarkers & Prev. 18(5):1386-9 (2009)), tumor cell migration (Wolf-Yadlin, A., et al., Molecular Systems Biology 2:54 (2006)), and the expression of proangiogenic factors (Wen, X. F., et al., Oncogene 25:6986-96 (2006)), suggesting a critical role for HER2 in promoting a tumorigenic environment. Although HER2-targeted therapies (i.e., Herceptin®/trastuzunab), in combination with chemotherapy, have significantly improved survival in HER2^{pos} BC patients (Piccart-Gebhart, M. J., et al., N. Eng. J. Med. 353(16):1659-72 (2005) (“Piccart-Gebhart, et al.”), a substantial proportion of patients become resistant to such therapies (Pohlmann, P. R., et al., Clin. Can. Res. 15:7479-91 (2009) (“Pohlman, et al.”)). Strategies to identify patient subgroups at high risk of treatment failure, as well as novel approaches to improve response rates to HER2-targeted therapies, are needed.

Growing evidence indicates that robust cellular immune responses in the tumor microenvironment are associated with improved outcomes in BC, particularly in the HER2^{pos} subtype. See, Alexe., G., et al., Can. Res. 67:10669-76 (2007). To that end, progress has been made in deciphering the individual immune mediators of these antitumor effects. Although cytotoxic CD8 T lymphocytes (“CTL”) were historically considered the primary effectors of antitumor immunity (Mahmoud, S. M., et al., J. Clin. Oncol. 29:1949-55 (2011)), boosting CTL responses with peptide vaccines in HER2-driven BC has yielded minimal clinical impact (Amin., A., et al., Cancer Immunol. Immunother. 57(12): 1817-25 (2008)), possibly because CTLs function suboptimally without adequate CD4⁺ T-lymphocyte help as reported by Bos, R., et al., Cancer Res. 70:8368-77 (2010). In addition to being critical for the generation and persistence of CTLs, CD4⁺ T-helper (“Th”) cells mediate antitumor effects through other mechanisms, including direct cytotoxic tumoricidal activity, modulation of antitumor cytokine responses, and potentiation of long-term immunologic memory (Cintolo, J. A., et al., Future Oncol. 8:1273-99 (2012)). By facilitating immunoglobulin class switching, Th cells also contribute to antitumor humoral immunity and effector B-cell responses. See, Parker, D. C., et al., Arm. Rev. Immunol. 11:331-60 (1993) (“Parker, et al.”). Indeed, the infiltration of interferon (“IFN”)-γ producing CD4⁺ T-helper type 1 (“Th1”) cells in the tumor microenvironment is associated with improved prognosis in BC. See, Gu-Trantien, C., et al., J. Clin. Inv. 123:2873-92 (2013).

The role of systemic anti-HER2 CD4⁺ Th1 responses in HER2-driven breast tumorigenesis, however, remains unclear. There remains an unmet need for strategies to predict patient subgroups at high risk of treatment failure, as well as approaches to improve response rates to HER2-targeted therapy with trastuzumab and chemotherapy. Thus, one or more present embodiments are directed to addressing one or more of the problems identified herein.

### BRIEF SUMMARY

In one broad aspect, there is provided a method for diagnosing or treating a mammalian subject having, or at risk of developing cancer, comprising: generating a circulating anti-cancer CD4⁺ Th1 response from antigen-presenting cells (“APCs”) or their precursors and CD4⁺ T-cells from a sample of the subject's blood which causes secretion of interferon-gamma (“IFN-γ”); and detecting the anti-cancer CD4⁺ Th1 response to determine if the response is depressed.

In another aspect, the generating step further comprises: isolating unexpanded peripheral blood mononuclear cells (“PBMCs”) from the blood sample; and pulsing the PBMCs and APC-precursor monocytes therein with a composition comprising immunogenic MHC class II binding peptides based on the type of cancer that afflicts the subject, thereby activating CD4⁺ Th1 cells in the PBMC's to secrete IFN-γ; and the detection step comprises detecting the secreted IFN-γ.

In an alternative aspect, the generation step further comprises: co-culturing purified CD4⁺ T-cells from the subject sample with APC immature or mature dendritic cells (“DCs”) from the subject sample pulsed with a composition comprising immunogenic MHC class II binding peptides based on the type of cancer that afflicts the subject, thereby activating the CD4⁺ T-cells to secrete IFN-γ; and the detection step comprises detecting the secreted IFN-γ.

In another aspect, the cancer is selected from the group consisting of breast, brain, bladder, esophagus, lung, pancreas, liver, prostate, ovarian, colorectal, and gastric cancer or any combination thereof.

In another aspect, the cancer is HER2-expressing.

In a further aspect, the cancer is HER2-positive breast cancer, the subject is a human female, and the immunogenic MHC class II binding peptides are based on the HER2 molecule

In preferred embodiments, the composition further comprises HER2 MHC class II antigen binding peptides which comprise:
Peptide 42-56 (SEQ ID NO: 1); Peptide 98-114 (SEQ ID NO: 2); Peptide 328-345 (SEQ ID NO: 3); Peptide 776-790 (SEQ ID NO: 4); Peptide 927-941 (SEQ ID NO: 5); and Peptide 1166-1180 (SEQ ID NO: 6).

In preferred embodiments, the IFN-γ production is measured by IFN-γ enzyme-linked immunospot assay (“ELISPOT”).

In another aspect there is a method for restoring HER2-specific CD4⁺ Th1 immune response in a HER2-positive breast cancer patient in need thereof, comprising: administering to the patient a therapeutically effective amount of a DC vaccine comprising autologous DCs pulsed with immunogenic HER2 MHC class II binding peptides (“DC vaccination”) to elevate the patient's anti-HER2 CD4⁺ Th1 response; and measuring the anti-HER2 CD4-Th1 response of the patient pre- and post-DC vaccination according to the generating and detecting steps of the above aspects to determine the amount of increase in the response, wherein the method for restoring further comprises: measuring the status of the anti-HER2 CD4⁺ Th1 response restoration of the patient post-DC vaccination by conducting the generating and detecting steps of the above aspects at one or more additional time intervals to monitor said response restoration.

In another aspect, there is a method for screening individuals for breast or other cancer, comprising: detecting anti-HER2 CD4⁺ Th1 responses of the individuals according to the method of the generating and detecting steps of the above aspects to determine if the responses are depressed as compared to healthy individuals.

In another aspect, there is a method for screening individuals at risk for developing breast or other cancer, comprising: detecting anti-HER2 CD4⁺ Th1 responses of the individuals according to the method of the generating and detecting steps of the above aspects to determine if the responses are depressed as compared to healthy individuals.

In another aspect, there is a method for predicting whether a patient with HER-positive breast cancer will respond well to standard non-immune therapy such as chemotherapy and trastuzumab, comprising: detecting the anti-HER2 CD4⁺Th1 response of the patient according to the method of the generating and detecting steps of the above aspects.

In another aspect, there is a method of predicting new breast events in HER2-positive-invasive breast cancer (“HER2^{pos}-IBC”) patients treated with trastuzumab and chemotherapy, comprising: measuring the anti-HER2 CD4⁺Th1 response of the patient according to the method of the generating and detecting steps of the above aspects to determine if said response is depressed.

In another aspect there is a method of predicting pathologic response of HER2-positive breast cancer following neoadjuvant trastuzumab and chemotherapy (“T/C”) therapy in a HER2-positive breast cancer patient, comprising: measuring the degree of anti-HER2 CD4⁺ Th1 responsiveness in said patient post-T/C treatment according to the method of the generating and detecting steps of the above aspects to determine if said response is a significantly higher anti-HER2 CD4⁺ Th1 response associated with neoadjuvant pathological complete response (no residual invasive breast cancer on postoperative pathology) or a lower response associated with non-pathological complete response and further, wherein in the case of a non-pathological complete response in said patient, the anti-HER2 CD4⁺ Th1 response of said patient is restored by DC vaccination.

In another broad aspect there is a method for diagnosing or treating a mammalian subject having, or at risk of developing cancer, comprising: obtaining blood from the subject; performing a blood test thereon which measures suppression in anti-cancer CD4+ Th1 response, and in the case of suppression; administering to the subject a cancer medicament in an effective amount selected from the group consisting of DC vaccine, targeted cancer therapy such as trastuzumab, conventional cancer therapy such as chemotherapy, surgery, and radiation.

In another aspect there is a method of monitoring a HER2^{pos}-IBC patient following completion of a targeted breast cancer therapy plus chemotherapy to assess the risk of recurrence of said cancer, comprising: measuring the degree of anti-HER2 CD4⁺ Th1 responsiveness in said patient post-therapy according to the method of the generating and detecting steps of the above aspects to determine if said response is a significantly depressed anti-HER2 CD4⁺ Th1 response that correlates with recurrence of said cancer or a higher anti-HER2 CD4⁺ Th1 response that correlates with non-recurrence. In a further aspect, said targeted therapy comprises administration of the drug trastuzumab to said patient.

Another broad aspect is an immune therapy for restoration of the pre-existing component of a subject's immunity that is lost upon the development of cancer in the subject, wherein the pre-existing component of immunity that is lost upon the development of cancer in the subject is anti-HER2 TH1 immune response. In this aspect, the subject has HER2^{pos} DCIS or HER2^{pos} IBC breast cancer and further the subject's anti-HER2 TH1 immune response is restored via administration of HER2-pulsed DC1 vaccine.

For a better understanding of exemplary embodiments, together with other and further features and advantages thereof, reference is made to the following description, taken in conjunction with the accompanying drawings, and the scope of the claimed embodiments will be pointed out in the appended claims.

### BRIEF DESCRIPTION OF THE DRAWINGS

The following detailed description of preferred embodiments will be better understood when read in conjunction with the appended drawings. For the purpose of illustrating the embodiments, there are shown in the drawings embodiments which are presently preferred. It should be understood, however, that the preferred embodiments are not limited to the precise arrangements and instrumentalities of the embodiments shown in the drawings.

FIG. 1 is a hierarchy diagram representing patient/donor groups included in the Reference Example study described herein. Cohorts are labeled A-H. Treatment schedules in cohorts G and H, as well as time-points at which blood was drawn are indicated in red callout boxes. Specifically, in the T/C-treated HER2^{pos}-IBC cohort (G), patients received either neoadjuvant T/C, followed by surgery and completion of adjuvant trastuzumab; patients selected for a surgery-first approach completed adjuvant TIC. Blood was drawn either <6 months or ≧6 months from completion of adjuvant trastuzumab.

FIG. 2 shows dendritic cell (“DC”) vaccination strategy. Patients' monocytes are first separated from other white blood cells by leukapheresis and elutriation. These monocytes are then cultured in serum-free medium (“SFM”) with granulocyte-macrophage colony-stimulating factor (“GM-CSF”) and interleukin (“IL”)-4 to become immature dendritic cells (“IDCs” or “iDCs”). These cells are then pulsed with six HER2 MHC class II binding peptides, and interferon (“IFN”)-γ and lipopolysaccharide (“LPS”) are added to complete the maturing and activation process to achieve full DC activation to DC1s before injecting back into the patient. See, Fracol, M., et al., Ann. Surg. Oncol. 20(10):3233 (2013). In the case of HLA-A2^{pos} patients, half of the cells were pulsed with a MHC class I binding peptide and the other half with a different MHC class 1 binding peptide.

FIGS. 3A and 3B are graphs showing inter-assay precision of ELISPOT. For the FIG. 3A studies, three parallel replicates over three days were run for samples from five donors (represented by different symbols) with known varying anti-HER2 reactivity in ELISPOT assays. The mean coefficient of variance (“Mean CV”) was plotted against cumulative anti-HER2 Th1 response (“Mean SFC (“spot forming cells”)/2×10⁵ cells”) for donors stimulated ex vivo with a HER2 extracellular domain (“ECD”) peptide mix (peptide 42-56 (SEQ ID NO: 1), peptide 98-114 (SEQ ID NO: 2), and peptide 328-345 (SEQ ID NO: 3)). Error bars represent standard deviation (“SD”) of the replicates. FIG. 3B shows the standard deviation (“SD”) of three assays on separate days plotted against cumulative Th1 response (“Mean SFC/2×10⁵ cells”) for each donor (represented by different symbols) as a measure of inter-assay variability. The connecting line represents linear regression of the SD generated, with 95% confidence intervals of the regression shown with parallel dotted lines.

FIG. 4 shows graphs which show the linearity of ELISPOT. Triplicate samples of peripheral blood mononuclear cells (“PBMCs”) from two high-responding HER2-reactive donors (DONOR #1, (triangles) and DONOR #2, (circles)) were serially diluted into PBMCs from a known allogeneic non-HER2 responder (same PBMC donor for all assays), and stimulated ex vivo with a HER2 ECD peptide mix (peptide 42-56 (SEQ ID NO: 1), peptide 98-114 (SEQ ID NO: 2), and peptide 328-345 (SEQ ID NO: 3)). Unstimulated background was subtracted for each dilution point in the ELISPOT assays.

FIGS. 5A-5D show anti-HER2 CD4⁺ Th1 response and IgG1/IgG4 reactivity are progressively lost in HER2^{pos} breast tumorigenesis. FIG. 5A shows histograms (left panels) of IFN-γ ELISPOT analysis of systemic CD4⁺ T-cells and anti-HER2 CD4⁺ Th1 response; corresponding post-hoc Scheffé p-value comparisons between patient groups are shown alongside the histograms (right panels). The patient groups studied were: HD (healthy donors); BD (benign breast biopsy); HER2^{neg} DCIS; HER2^{neg} IBC (non-equivocal HER2^{neg} (HER2 0 and 1+) invasive breast cancer); HER2^{pos} DCIS (HER2^{pos} ductal carcinoma in situ); and HER2^{pos} IBC (Stage I/II HER2^{pos} invasive breast cancer). The top histogram shows overall anti-HER2 responsivity (%100) (percentage of patients responding to ≧1 reactive peptide) (also referred to as “anti-HER2 responsivity”); the middle histogram shows mean number of reactive peptides (n) (the mean number of reactive peptides (“n”) the patients in the group reacted to as a whole) (also referred to as “response repertoire”); and the bottom histogram shows mean total SFC/10⁶ cells (total sum of reactive spots (spot-forming cells “SFC” per 10⁶ cells from IFN-γ ELISPOT analysis) from all 6 MHC Class II binding peptides from each subject group) (also referred to as “cumulative response”) (all ANOVA p<0.001). A progressive loss of CD4⁺ Th1 response in HER2^{pos} breast tumorigenesis is shown (i.e. HD/BD→HER2^{pos}-DCIS→HER2^{pos}-IBC) when assessed by anti-HER2 responsivity, response repertoire, and cumulative response. No differences in Th1 responses were found between HER2^{neg}-DCIS and HER2^{neg}-IBC (IHC 0/1+) and HD/BD subjects. FIG. 5B shows IFN-γ production by ELISPOT (cumulative response (mean total SFC/2×10⁵ cells)) in the same respective patient groups as in FIG. 5A, with the addition of the T/C-treated HER2-IBC patient group (“T/C” means trastuzumab and chemotherapy). Results are presented as median±interquartile range (“IQR”) IFN-γ SFC per 2×10⁵ cells in box- and whiskers plots. FIG. 5C shows histograms for variations in anti-HER2 Th1 cumulative responses in HD/BDs stratified by donor age (<50 years v.≧50 years) (upper left panel), menopausal status (pre-menopausal v. post-menopausal) (upper right panel), race (white v. other) (lower left panel) and gravidity (zero v.≧1 pregnancies) (lower right panel) Within each Th1 metric, results are expressed as proportion or mean (±SEM). FIG. 5D shows ELISA results of serum reactivity against recombinant HER2 ECD peptides. ELISA measurements are shown as optical density (“OD”) at 1:100 sera dilutions (grouped scatter plot, with horizontal lines indicating mean OD). Anti-HER2 IgG1 antibody levels (top panel) and anti-HER2 IgG4 antibody levels (bottom panel) were measured in HD (circles/left), HER2^{pos}-DCIS (squares/middle), and HER2^{pos}-IBC (triangles/right) patients (***p<0.001 by unpaired t-test or ANOVA with post-hoc Scheffé testing, as applicable). Significantly elevated anti-HER2 IgG1 and IgG4 antibody levels were present in HER2^{pos}-DCIS patients compared with HDs, that decayed in HER2^{pos}-IBC patients.

FIG. 6 shows individual HER2 peptide contributions to cumulative CD4⁺ Th1 immunity in HER2^{pos} breast tumorigenesis for HD (healthy donors); BD (benign breast biopsy); HER2^{neg} DCIS; HER2^{neg} IBC (non-equivocal HER2^{neg} (HER2 0 and 1+) invasive breast cancer); HER2^{pos} DCIS (HER2^{pos} ductal carcinoma in situ); and HER2^{pos} IBC (Stage I/II HER2^{pos} invasive breast cancer) patients do not reflect immune sculpting. HER2 extracellular domain (“ECD”)-restricted peptides and intracellular domain (“ICD”)-restricted peptides were used. Th1 reactivity profiles are shown for ECD peptide 42-56 (“ECD p42-56”) (SEQ ID NO: 1) (top left); ECD peptide 98-114 (“ECD p98-114”) (SEQ ID NO: 2) (middle left) and ECD peptide 328-345 (“ECD p328-345”) (SEQ ID NO: 3) (bottom left) and for ICD peptide 776-790 (“ICD p776-790”) (SEQ ID NO: 4) (top right); ICD peptide 927-941 (“ICD p927-941”) (SEQ ID NO: 5) (middle right), and ICD peptide 1166-1180 (“ICD p1166-1180”) (SEQ ID NO: 6) (bottom right). Individual peptide-specific responses are depicted as mean IFN-γ SFC per 2×10⁵ PBMCs by ELISPOT. Th1 reactivity profiles show a significant stepwise decline in anti-HER2 Th1 immunity across a continuum (HD→BD→HER2^{neg}-DCIS→HER2^{neg}-IBC→HER2^{pos}-DCIS→HER2^{pos}-IBC) in HER2^{pos} breast tumorigenesis (all p<0.005 by ANOVA). Results are expressed as mean±SEM.

FIG. 7 shows minimal temporal variability in donor anti-HER2 Th1 responses. Donor-matched anti-HER2 Th1 cumulative response (left panel) and response repertoire (right panel), generated from blood samples obtained at least 6 months apart, are plotted for paired HD (green triangles; n=4) and treatment-naïve HER2^{pos}-IBC subjects (blue squares; n=4). Minimal within-donor Th1 response variability was observed in both HD and treatment-naïve HER2^{pos}-IBC subjects over time (all p=NS).

FIGS. 8A-8E show anti-HER2 Th1 deficit in HER2^{pos}-IBC is not attributable to lack of immunocompetence or increase in immunosuppressive phenotypes, but is associated with a functional shift in IFN-γ:IL-10-producing phenotypes. FIG. 8A shows IFN-γ production by measuring cumulative Th1 response (mean total SFC/10⁵ cells) to recall stimuli tetanus toxoid or Candida albicans in IFN-γ ELISPOT. Results are presented as median±interquartile range (IQR) IFN-γ SFC per 2×10⁵ cells in box-and-whiskers plots. PBMCs from HER2^{pos}-IBC patients, both treatment-naïve and T/C treated, did not differ significantly from those of HDs. FIG. 8B, top panels, show representative flow cytometry stainings using PBMCs from HD, HER2^{pos}-IBC (Stage I/II) and HER2^{pos}-IBC s/p T/C (patient T/C-treated) patients to determine their immunophenotype. Relative proportions of CD4⁺ (CD3⁺CD4⁺) (top stainings) or CD8⁺ (CD3⁺CD8⁺) T-cells (bottom stainings) are shown and are represented in the bottom histograms which show respectively, relative proportions of CD4⁺ (CD3⁺CD4⁺) T-cells (left) and CD8⁺ (CD3⁺CD8⁺) T-cells (right) for the patient groups: HD (dark bars), Stage I/II HER^{pos}-IBC (medium bars) and HER2^{pos}-IBC s/p T/C (light bars). PBMCs from HER2^{pos}-IBC patients, both treatment-naïve and T/C treated, did not differ significantly from those of HDs. FIG. 8C, top panels, show representative flow cytometry stainings using PBMCs from HD, HER^{pos}-IBC (Stage I/II) and HER2^{pos}-IBC s/p T/C to determine their immunophenotype. Relative proportions of regulatory T-cells (“T_{reg}”; CD4⁺CD25⁺FoxP3⁺) (top stainings) and myeloid-derived suppressor cells (“MDSC”; CD11b⁺CD33⁺HLA-DR⁻ CD83⁻) (bottom stainings) are shown and are represented in the lower histograms which show respectively relative proportions of, regulatory T-cells (T_{reg}; CD4⁺CD25⁺FoxP3⁺) (left) or myeloid-derived suppressor cells (“MDSC”; CD11b⁺CD33⁺ HLA-DR⁻ CD83⁻) (right) for the patient groups: HD (dark bars), Stage I/II HER^{pos}-IBC (medium bars) and HER2^{pos}-IBC s/p T/C (light bars). PBMCs from HER2^{pos}-IBC patients, both treatment-naïve and T/C treated, did not differ significantly from those of HDs. FIG. 8D shows circulating HER2-specific IL-10 production does not vary between patient groups. PBMCs from HER2^{pos}-IBC patients, both treatment-naïve (HER2^{pos}-IBC) and those receiving T/C (HER2^{pos}-IBC s/p T/C), did not differ significantly from HDs in anti-HER2 IL-10 production via ELISPOT, assessed by overall anti-HER2 responsivity (top), repertoire (middle), and cumulative response. (bottom). Results are expressed as proportion or mean±SEM. FIG. 8E shows relative HER2-specific IFN-γ and IL-10 production in HER2^{pos} breast tumorigenesis. Donor-matched cumulative IFN-γ production and IL-10 production (SFC/10⁶ cells) across six HER2 Class II peptides in HD, HER2^{pos}-IBC (treatment-naïve), and HER2^{pos}-IBC s/p T/C (T/C-treated) patients were compared. The bar graphs show the relative HER2-specific IFN-γ to IL-10 proportions via percentage of SFC contribution (% depicted in graphs) across the patient groups for HER2 antigen-specific reaction (top panel) and positive control (CD3 or CD8/28) (bottom panel); (IFN-γ production (green); IL-10 production (red)). Relative HER2-specific IFN-γ to IL-10 proportions decreased significantly from HDs to HER2^{pos}-IBC patients with or without T/C-treatment. Absolute IFN-γ:IL-10 production ratio changed from 6.6:1 (HDs) to 0.97:1 (T/C-treated) and 0.74:1 (HER2^{pos}-IBC), respectively (top panel). No significant relative shifts in IFN-γ:IL-10 production were observed to positive controls (anti-CD3/anti-CD3/CD28) (bottom panel).

FIGS. 9A-9B show systemic loss in anti-HER2 CD4⁺ TH1 subsets is not related to disproportionate peritumoral T lymphocyte trafficking in HER2^{pos} breast lesions. FIG. 9A shows two photographs of representative hematoxylin and eosin (“H&E”) stainings of tissue samples from HER2^{pos}-DCIS lesions (top) and HER2^{pos}-IBC tumors (bottom) (magnification bars 25 μm). The arrows point to a relative paucity of lymphocytic infiltrate observed in the peritumoral stroma of HER2^{pos}-IBC tumors (bottom) as compared with HER2^{pos}-DCIS lesions (top) by immunohistochemical staining. Stromal lymphocyte infiltration in evaluable HER2^{pos}-DCIS (n=14) and HER2^{pos}-IBC (n=8) is quantified as low (<15% involvement), moderate (15-24%) and high (≧25%) in the adjoining table. FIG. 9B shows four photographs of the results of multiplex-labeled immunofluorescence in representative HER2^{pos}-DCIS (left) and HER2^{pos}-IBC (right) lesions. A striking paucity of CD4⁺ T-cells (green signal) was observed in 5/5 (100%) HER2^{pos}-IBC tumors, where the predominant infiltrating and stromal lymphocytic infiltrate is CD8⁺ (yellow signal). By comparison, a predominantly CD4⁺ T-cell infiltrate was seen in DCIS-containing ducts (4/4 tumors). Representative HER2^{pos}-DCIS and IBC lesions are depicted; multiplexed-labeled images are shown above corresponding H&E sections (magnification bar 25 μm).

FIGS. 10A-10D show CD4⁺ Th1 induces apoptosis of HER2^{high}, but not HER2^{low}, human and murine breast cancer cells. FIG. 10A shows (top panels) photographic results of western blot analysis for detection of cleaved caspace-3. SK-BR-3 cells were co-cultured with: Lane 1)—complete medium alone (complete medium); Lane 2)—10⁶ CD4⁺ T-cells alone (CD4⁺ only); Lanes 3 and 4)—10⁶ CD4⁺ T-cells plus 10⁵ HER2 Class II peptide (“iDC H”)—or irrelevant Class II BRAF peptide (“iDC B”)-pulsed immature DCs (“iDCs”), respectively; Lanes 5 and 6)—10⁶ CD4⁺ T-cells plus 10⁵ each HER2 (“DC1 H”)—or BRAF (“DC1 B”)-pulsed DC1s, respectively; Lane 7)—CD4⁺ 10⁶ DC1 H 10⁵+IFN-γ & TNF-α neutr Ab and Lane 8)-CD4⁺ 10⁶ DC1 H 10⁵+IgG isotype control Ab. Increased caspase-3 cleavage indicated dose-dependent apoptosis of SK-BR-3 cells when co-cultured with DC1 H:CD4⁺ T-cells, but not DC1 B, iDC H, or iDC B groups. Vinculin was used as a loading control. The displayed western blot is representative of three experiments. The middle panel bar graph (red bars) shows results expressed as mean caspace-3/vinculin ratios±SEM indicating fold induction of apoptosis (quantified using ImageJ software) that corresponds to western blot Lanes 1-6 in the top panel. In the bar graph to the right (black bars) (corresponding to western blot Lanes 7-8 in the top panel) the bars represent % rescue of apoptosis/mean caspase-3/vinculin ratio±SEM (31.415.3% IFN-γ/TNF-α neutralization vs. control) over three experiments. Compared with IgG isotype control, CD4⁺:DC1 H-induced SK-BR-3 apoptosis was significantly rescued by neutralizing IFN-γ and TNF-α. The bottom panel shows corresponding production of IFN-γ (left y-axis) (solid bars) and TNF-α (right y-axis) (lined bars) in respective co-cultures by ELISA. Results are expressed in pg/mL, and are representative of three experiments. FIG. 10B shows photographs of the cells of the “CD4⁺ only,” “CD4⁺+DC1 B”, and “CD4⁺+DC1 H,” cell groups. Apoptotic cells were revealed by DAPI staining. In the CD4⁺+DC H group, a greater number of apoptotic cells (asterisks) were observed when compared with CD4⁺+DC1 B or CD4⁺ only groups. The bar graph (right) shows % apoptotic cells (fold induction) of apoptotic cells for the three cell groups pictured, with a 25-fold increase in apoptosis for the CD4⁺+DC H group that correlates with the visual results. Results are representative of three experiments, and expressed as mean % apoptotic cells±SEM. FIG. 10C shows photographs of the results of western blot analysis in which HER2^{high} SK-BR-3, HER2^{intermediate} MCF-7, and HER2^{low} MDA-MB-231 human BC cells uniformly maintained expression of IFN-γ-Rα and TNF-α-R1 receptors. Vinculin was used as a loading control. FIG. 10D shows that in transgenic murine HER2^{high} mammary carcinoma TUBO (top graph) and MMC15 (HER2^{high}) cells (middle graph), combination treatment with recombinant murine (“rm”) Th1 cytokines rmIFN-γ and rmTNF-α resulted in significantly greater apoptosis compared with untreated controls (no Rx) or treatment with either cytokine alone. This effect was not reproduced with dual rmIFN-γ+rmTNF-α treatment in murine HER2^{low/neg} cells 4T1 (bottom graph). Results are representative of three experiments, and expressed as mean % apoptotic cells±SEM, detected by proportion of PI^{pos}/Annexin V^{pos} cells by flow cytomety. (*p≦0.05, **p<0.01, ***p<0.001).

FIGS. 11A-11C show HER2^{high}, but not HER2^{low}, human BC cells are sensitive to CD4⁺ Th1-mediated apoptosis, by virtue of Th1-elaborated cytokines IFN-γ and TNF-α. FIG. 11A shows (top panels) photographic results of western blot analysis for detection of cleaved caspace-3. Using a transwell system, 50×10³ MCF-7 (HER2^{intermediate}) and 50×10 MDA-MB-231 (HER2^{low}) cells were co-cultured with medium alone (complete medium), 10⁶ CD4⁺ T-cells alone (CD4⁺ only), and 10⁶ CD4⁺ T-cells+10⁵ each HER2 (DC1 H)- or BRAF control (DC1 B)-pulsed DC1s. Caspase-3 cleavage shown in the western blots and represented in the corresponding bar graphs below (lower panel) indicated increased apoptosis of MCF-7 (left panels), but not MDA-MB-231 cells (right panels) when co-cultured with DC1 H:CD4⁺ T-cells. Vinculin was used as loading control. The displayed western blots are representative of three experiments, and results are expressed as mean caspase-3/vinculin ratios±SEM (indicating fold induction of apoptosis FIG. 11B shows photographs of western blot results of co-culturing SK-BR-3 cells with the supernatants from the following treatment conditions in FIG. 10A [complete medium alone; 10⁶ CD4⁺ T-cells alone (CD4⁺ only); CD4⁺ T-cells+HER2-pulsed iDC (“iDC H”); CD4⁺ T-cells+BRAF-pulsed iDC (“iDC B”); CD4⁺ T-cells+10⁵ HER2-pulsed DC1 (“DC1 H”); and CD4⁺ T-cells+10⁵ BRAF-pulsed DC1 (“DC1 B”)] were co-cultured with 50×10 SK-BR-3 cells. Relatively higher cleaved caspase-3 levels were detected in the DC1 H:CD4 group compared with DC1 B, iDC H, iDC B, or CD4⁺ only groups. Results are representative of three experiments. FIG. 11C shows photographs of western blot results (top panels) of culturing SK-BR-3 (left), MCF-7 (center), and MDA-MB-231 cells (right) with indicated amounts of TNF-α and IFN-γ for detection of cleaved caspace-3. The bars of the lower panel bar graph correspond to the lanes of the western blot displayed in the top panels. Combination treatment with Th1 cytokines IFN-γ and TNF-α resulted in greater apoptosis in SK-BR-3 (HER2^{high}; 10 ng/mL TNF-α+100 U/mL IFN-γ) and MCF-7 (HER2^{intermediate}; 100 ng/mL TNF-α+1000 U/mL IFN-γ) cells, compared with untreated controls. MDA-MB-231 cells (HER2^{low}; 200 ng/mL TNF-α+2000 U/mL IFN-γ) remained largely unaffected by dual IFN-γ+TNF-α treatment. Results are representative of three experiments. (*p≦0.05, **p<0.001).

FIGS. 12A-12E show anti-HER2 CD4⁺ Th1 immunity is differentially restored following HER2-pulsed DC1 immunization, but not after HER2-targeted therapies. FIG. 12A is a graph of CD4⁺ Th1 responses in treatment-naïve HER2^{pos}-IBC patients (“HER2^{pos}-IBC no tx”) (black) and HER2^{pos}-IBC patients receiving trastuzumab and chemotherapy (“t/C-treated HER2^{pos}-IBC”) (red), assessed by overall anti-HER2 responsivity (top), response repertoire (middle), and cumulative response (bottom). Compared with treatment-naïve Stage I/II HER2^{pos}-IBC patients (no tx), anti-HER2 Th1 responses were not globally augmented following T/C treatment in stage I-III HER2^{pos}-IBC patients (T/C-treated), illustrated by anti-HER2 responsivity (top), repertoire (middle), or cumulative response (bottom). The relative proportion of IFN-γ:IL-10 reactive cells (% depicted in lower panel histograms; IFN-γ: solid; IL-10: diagonal lines) following HER2-specific and tetanus (positive control) stimuli did not improve in T/C-treated (n=5) compared with no tx (n=5). FIG. 12B is a graph of CD4 Th1 responses in HER2-IBC patients immediately prior to and following HER2 pulsed-DC1 immunization (“HER2^{pos}-IBC PRE vax”) (black) and (“HER2^{pos}-IBC POST vax”) (green) respectively, assessed by overall anti-HER2 responsivity (top), response repertoire (middle), and cumulative response (bottom). Significant improvements in all anti-HER2 Th1 immune metrics were observed in 11 Stage I HER2^{pos}-IBC (PRE vax) patients immediately following HER2 pulsed-DC immunization (POST vax). While relative proportion of IFN-γ to IL:10 reactive cells (% depicted in lower panel histograms-IFN-γ: solid; IL-10: diagonal lines) did not change appreciably following tetanus stimulation, HER2-pulsed vaccination significantly increased the relative proportion of IFN-γ to IL: 10 reactive cells in POST vax (n=5) compared with PRE vax (n=5) patients. FIG. 12C shows stage-matched effects of DC vaccination and trastuzumab/chemotherapy on anti-HER2 Th1 immunity. Matched comparison between AJCC Stage I treatment-naïve (“No tx”), T/C-treated (“TIC-treated”), and HER2-pulsed DC1 immunization (“POST-vax”) HER2^{pos}-IBC patients were assessed by overall anti-HER2 responsivity (top), response repertoire (middle), and cumulative response (bottom). The differential Th1 restoration following HER2-pulsed DC1 immunization, but not T/C treatment, persisted on stage-matched comparisons in Stage I HER2^{pos}-IBC patients. Results are expressed as proportion or mean±SEM; (**p<0.01, ***p<0.001). FIGS. 12D and 12E show the durability of CD4⁺ Th1 immune response after DC vaccination. Immune responses in were compared in Stage I/II HER2^{pos}-IBC patients pre-DC vaccination (“PRE VACCINE”), immediately after DC vaccination (“IMMEDIATE POST VACCINE”) and ≧6 months after vaccination (“≧6 MO POST VACCINE”). Beyond the immediate post-vaccination period, anti-HER2 CD4⁺ Th1 immunity remained durably augmented in 9 of II evaluable patients≧6 months following vaccination, despite initiation/completion of systemic chemotherapy in all patients by this time-point (broken arrows). Scatter plots demonstrate CD4⁺ Th1 reactivity profiles by response repertoire (FIG. 12D) and cumulative response (FIG. 12E) for individual vaccinated subjects.

FIGS. 13A-13E show depressed anti-HER2 Th1 responses following T/C treatment correlate with adverse clinical and pathologic outcomes. The graphs of FIGS. 13A-13D show subgroup analysis of T/C-treated HER2^{pos}-IBC patients demonstrated no appreciable differences in anti-HER2 responsivity (top graphs), repertoire (middle graphs), or cumulative response (bottom graphs) when stratified by FIG. 13A—sequencing of chemotherapy (neoadjuvant vs. adjuvant); FIG. 13B-time from completion of trastuzumab to enrollment in study (<6 vs. ≧6 months); FIG. 13C—estrogen-receptor status (ER^{pos} vs. ER^{neg}) and FIG. 13D—pathologic stage (I vs. II vs. III). FIG. 13E shows that compared with HER2^{pos}-IBC patients who did not incur breast events (“No BE”) following completion of T/C, patients incurring BEs (“+BE”) had significantly depressed anti-HER2 responsivity (left top graph) and cumulative Th1 responses (bottom left graph). In HER2^{pos}-IBC patients achieving pathologic complete response (pCR) following neoadjuvant T/C, anti-HER2 Th1 response repertoire (right middle graph) and cumulative response (right bottom graph) was significantly greater compared to non-pCR patients.

FIG. 14 is a flow diagram of study populations for Experimental Example 2 in which 95 HER2^{pos} breast cancer patients were enrolled. All tumors were histologically confirmed invasive breast cancer with HER2 overexpression (3+ or 2+/FISH-positive). Time-points at which blood was drawn are indicated in red callout boxes. Median follow-up in the trastuzumab and chemotherapy (T+C)-treated cohort was 44 (IQR 31) months.

FIGS. 15A-15D show variation in anti-HER2 CD4⁺ Th1 response of HER2^{pos}-IBC non-recurrence and recurrence patient cohorts. FIG. 15A shows IFN-γ⁺ anti-HER2 CD4⁺ T-cell response variations between HER2^{pos}-IBC breast cancer patient cohorts (treatment-naïve [n=22], recurrence [n=25], and non-recurrence [n=48]), stratified by anti-HER2 responsivity (top panel), response repertoire (middle panel), and cumulative response (lower panel). Results are expressed as proportion (%) or mean±SEM. One-way ANOVA p-values are shown alongside; *p<0.05, ***p<0.001 on post-hoc Bonferroni testing. FIG. 15B shows PBMCs from non-recurrence and recurrence cohorts did not differ significantly in immune competence—measured by IFN-γ production to anti-CD3/anti-CD28 stimulus (top panel) or recall stimuli tetanus toxoid (bottom left panel) and Candida albicans (bottom right panel)—by ELISPOT. Results are presented as median±interquartile range (“IQR”) IFN-γ SFC/2×10⁵ cells. FIG. 15 C shows relative contributions of Th1 (T-bet⁺IFN-γ⁺) “T-bet” versus Th2 (GATA-3⁺IFN-γ⁺) “GATA-3” phenotypes to HER2 peptide-specific IFN-γ⁺ cells in PBMCs of non-recurrence and recurrence cohorts. Representative stainings within these groups are shown after gating on CD4⁺ cells (top panel); results in adjoining histograms are expressed as mean proportions (%)±SEM as indicated (middle panel). The bottom panel shows circulating HER2-specific IL-4 production does not vary between non-recurrence and recurrence cohorts, when assessed by anti-HER2 Th1 responsivity (left), repertoire (center), and cumulative response (right). Results are expressed as proportion or mean±SEM. FIG. 15D shows relative proportions of Treg (CD4⁺CD25⁺FoxP3⁺) by flow cytometry. Representative stainings within groups are shown (top panel); results in adjoining histograms are expressed as mean proportions (%)±SEM (middle panel). The bottom panel shows HER2-specific IL-10 production is similar between non-recurrence and recurrence cohorts across all three Th1 metrics. Results are expressed as proportion or mean±SEM.

FIG. 16 is a graph showing Cox proportional hazards modeling of disease-free survival (“DFS”) in completely treated HER2^{pos} breast cancer patients, stratified by anti-HER2 Th1 responsivity. Non-Th1-responsive patients demonstrate markedly worse risk-adjusted DFS relative to Th1-responsive patients.

FIG. 17 is a schematic of a representative calcium mobilizing pre-treatment regimen, for example, calcium mobilizing pre-treatment of immature DC followed later with combinations of activating agents selected to amplify dendritic cell 3^{rd} signals leading to alterations in T cell phenotype. Immature monocyte-derived DCs are treated for 4 hours with calcium-mobilizing agents (ionophores). DCs are then exposed to additional activation agents. As seen by the upper route, to induce the production of the Th1-polarizing cytokine IL-12, immature DCs are activated with combinations selected from the cytokine IFN-γ, the TLR agonists bacterial LPS and R848. This induces the Th1 phenotype that produces IFN-γ. Alternatively, the bottom route for the Th-17 phenotype which secretes IL-17 and IL-22, begins with 4 hour calcium ionophore-pretreatment of immature DCs followed by activation with combinations selected from the TLR agonist LPS, ATP, bacterial LTA, and prostaglandin E2 (PGE2). This causes the third-signal agents IL-23, IL-1β, and IL-6 to be amplified, thereby inducing the Th17 phenotype which leads an immune response dominated by IL-17 and IL-22-secreting Th17 cells.

### DETAILED DESCRIPTION

It is to be understood that the figures, images and descriptions of the present embodiments have been simplified to illustrate elements that are relevant for a clear understanding, while eliminating, for the purposes of clarity, many other elements which may be found in the present embodiments. Those of ordinary skill in the pertinent art will recognize that other elements are desirable and/or required in order to implement the present embodiments. However, because such elements are well known in the art, and because such elements do not facilitate a better understanding of the present embodiments, a discussion of such elements is not provided herein.

Reference throughout this specification of “one embodiment” or “an embodiment” or the like means that a particular feature, structure, or characteristic described in connection with the embodiment is included in at least one embodiment. Thus appearances of the phrases “in one embodiment” or “in an embodiment” or the like in various places throughout this specification are not necessarily all referring to the same embodiment.

In addition, for the purpose of promoting an understanding of the principles of the present disclosure, reference will now be made to the embodiments shown and described herein, and specific language will be used to describe the same. It will, nevertheless, be understood that no limitation of the scope of the disclosure is thereby intended; any alterations and further modifications of the described or illustrated embodiments and any further applications of the principles of the disclosure as illustrated herein are contemplated as would normally occur to one skilled in the art to which the disclosure relates. All limitations of scope should be determined in accordance with and as expressed in the eventual claims of one or more issued patents.

### Definitions

Unless defined otherwise, all technical and scientific terms used herein have the same meaning as commonly understood by one of ordinary skill in the art to which the inventive subject matter of this disclosure belongs. Although any methods and materials similar or equivalent to those described herein can be used in the practice or testing of the present embodiments, the preferred methods and materials are described.

Generally, the nomenclature used herein and the laboratory procedures in cell culture, molecular genetics, organic chemistry, and nucleic acid chemistry and hybridization are those well-known and commonly employed in the art.

Standard techniques are used for nucleic acid and peptide synthesis. The techniques and procedures are generally performed according to conventional methods in the art and various general references (e.g., Sambrook and Russell, 2012, Molecular Cloning, A Laboratory Approach, Cold Spring Harbor Press, Cold Spring Harbor, N.Y., and Ausubel et al., 2012, Current Protocol in Molecular Biology, John Wiley & Sons, NY), which are provided throughout this document.

The nomenclature used herein and the laboratory procedures used in analytical chemistry and organic syntheses described below are those well-known and commonly employed in the art. Standard techniques or modifications thereof are used for chemical syntheses and chemical analyses.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, or ±10%, or ±5%, or ±1%, or ±0.1% from the specified value, as such variations are appropriate to perform the disclosed methods.

“Adjuvant therapy” for breast cancer as used herein refers to any treatment given after primary therapy (i.e., surgery) to increase the chance of long-term survival. “Neoadjuvant therapy” is treatment given before primary therapy.

The term “antigen” or “ag” as used herein is defined as a molecule that provokes an immune response. This immune response may involve either antibody production, or the activation of specific immunologically-competent cells, or both. One of ordinary skill in the art will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full-length nucleotide sequence of a gene. It is readily apparent that the present embodiments include, but are not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated or synthesized or can be derived from a biological sample. Such a biological sample can include, but is not limited to a tissue sample, a tumor sample, a cell or a biological fluid.

An “antigen presenting cell” or “APC” is a cell that is capable of activating T cells, and includes, but is not limited to, monocytes/macrophages, B cells and dendritic cells (“DCs”).

“Antigen-pulsed APC” or an “antigen-loaded APC” includes an APC which has been exposed to an antigen and activated by the antigen. For example, an APC may become Ag-loaded in vitro, e.g., during culture in the presence of an antigen. An APC may also be loaded in vivo by exposure to an antigen. An “antigen-loaded APC” is traditionally prepared in one of two ways: (1) small peptide fragments, known as antigenic peptides, are “pulsed” directly onto the outside of the APCs; or (2) the APC is incubated with whole proteins or protein particles which are then ingested by the APC. These proteins are digested into small peptide fragments by the APC and are eventually transported to and presented on the APC surface. In addition, an antigen-loaded APC can also be generated by introducing a polynucleotide encoding an antigen into the cell.

“Anti-HER2 response” is the immune response specifically against HER2 protein.

The term “anti-tumor effect” as used herein, refers to a biological effect which can be manifested by a decrease in tumor volume, a decrease in the number of tumor cells, a decrease in the number of metastases, an increase in life expectancy, or amelioration of various physiological symptoms associated with the cancerous condition. An “anti-tumor effect” can also be manifested by the ability of binding peptides, polynucleotides, cells and antibodies in prevention of the occurrence of tumor in the first place.

“Apoptosis” is the process of programmed cell death. Caspase-3 is a protease frequently activated during apoptotic cell death.

“ATP” means adenosine triphosphate, a high-energy molecule used for energy transfer within a cell. Damaged mammalian cells “leak” ATP, and some bacteria secrete it into their surroundings. Dendritic cells sense ATP as evidence of either cell damage or infection through purinergic receptors on their surface.

As used herein, the term “autologous” refers to any material derived from the same individual to which it is later to be introduced.

The term “B cell” as used herein is defined as a cell derived from the bone marrow and/or spleen. B cells can develop into plasma cells that produce antibodies, and can also serve as APC.

“Binding peptides.” See, “HER2 binding peptides.”

“Calcium ionophore” as used here refers to a class of drugs that renders the cell membrane permeable to calcium ions. In some instances, calcium ionophores keep the calcium concentrations in the cytoplasm of the cell artificially high, and can be used to activate cells, including DCs. Commonly used calcium ionophores include but are not limited to A23187 and ionomycin.

The term “cancer” as used herein is defined as a hyperproliferation of cells whose unique trait—loss of normal control—results in unregulated growth, lack of differentiation, local tissue invasion, and/or metastasis. Examples include but are not limited to, breast cancer, prostate cancer, ovarian cancer, cervical cancer, skin cancer, bladder cancer, esophageal cancer, pancreatic cancer, colorectal cancer, gastric cancer, renal cancer, liver cancer, brain cancer, lymphoma, leukemia, lung cancer, germ-cell tumors, and the like.

“CD4⁺ Th1 cells,” “Th1 cells,” “CD4⁺ T-helper type 1 cells,” “CD4⁺ T cells,” and the like are defined as a subtype of T-helper cells that express the surface protein CD4 and produce high levels of the cytokine IFN-γ. See also, “T-helper cells.”

The term “cryopreserved” or “cryopreservation” as used herein refers to cells that have been resuspended in a freezing medium and frozen at a temperature of around −70° C. or lower.

“Cumulative response” means the combined immune response of a patient group expressed as the total sum of reactive spots (spot-forming cells “SFC” per 10⁶ cells from IFN-γ ELISPOT analysis) from all 6 MHC class II binding peptides from a given patient group.

“DC vaccination,” “DC immunization,” “DC1 immunization,” and the like refer to a strategy using autologous dendritic cells to harness the immune system to recognize specific molecules and mount specific responses against them.

The term “dendritic cell” or “DC” is an antigen presenting cell existing in vivo, in vitro, ex vivo, or in a host or subject, or which can be derived from a hematopoietic stem cell or a monocyte. Dendritic cells and their precursors can be isolated from a variety of lymphoid organs, e.g., spleen, lymph nodes, as well as from bone marrow and peripheral blood. DCs have a characteristic morphology with thin sheets (lamellipodia) extending in multiple directions away from the dendritic cell body. Typically, dendritic cells express high levels of MHC and costimulatory (e.g., CD80 (B7-1) and CD86 (B7-2)) molecules. Dendritic cells can induce antigen specific differentiation of T cells in vitro, and are able to initiate primary T cell responses in vitro and in vivo. In the context of vaccine production, an “activated DC” is a DC that has been exposed to a Toll-like receptor agonist such as lipopolysaccharide “LPS.” An activated DC may or may not be loaded with an antigen. See also, “mature DC.”

“DC-1 polarized dendritic cells,” “DC1s” and “type-1 polarized DCs” refer to mature DCs that secrete Th1-driving cytokines, such as IL-12, IL-18, and IL-23. DC1s are fully capable of promoting cell-mediated immunity. DC1s are pulsed with HER2 MHC class II-binding peptides in preferred embodiments herein.

“Effective amount” or “therapeutically effective amount” are used interchangeably herein, and refer to an amount of a compound, formulation, material, or composition, as described herein effective to achieve a particular biological result. Such results may include, but are not limited to, the inhibition of virus infection as determined by any means suitable in the art.

“Estrogen receptor (“ER”) positive” or “ER^{pos}” cancer is cancer which tests positive for expression of estrogen. Conversely, “ER negative” cancer tests negative for such expression. Analysis of ER status can be performed by any method known in the art.

“HER2” is a member of the human epidermal growth factor receptor (“EGFR”) family. HER2 is overexpressed in approximately 20-25% of human breast cancer and is expressed in many other cancers.

“HER2 binding peptides,” “HER2 MHC class 1: binding peptides,” “binding peptides,” “HER2 peptides,” “immunogenic MHC class II binding peptides,” “antigen binding peptides,” “HER2 epitopes,” “reactive peptides,” and the like as used herein refer to MHC Class II peptides derived from or based on the sequence of the HER2/neu protein, a target found on approximately 20-25% of all human breast cancers and their equivalents. HER2 extracellular domain “ECD” refers to a domain of HER2 that is outside of a cell, either anchored to a cell membrane, or in circulation, including fragments thereof. HER2 intracellular domain “ICD” refers to a domain of the HER2/neu protein within the cytoplasm of a cell. According to a preferred embodiment HER2 epitopes or otherwise binding peptides comprise 6 HER2 binding peptides which include 3 HER2 ECD peptides and 3 HER2 ICD peptides.

Preferred HER2 ECD peptides comprise:

| | |
|---|---|
| Peptide 42-56: | |
| HLDMLRHLYQGCQVV; | (SEQ ID NO: 1) |
| Peptide 98-114: | |
| RLRIVRGTQLFEDNYAL; | (SEQ ID NO: 2) |
| and | |
| Peptide 328-345: | |
| TQRCEKCSKPCARVCYGL; | (SEQ ID NO: 3) |

Preferred HER2 ICD peptides comprise:

| | |
|---|---|
| Peptide 776-790: | |
| GVGSPYVSRLLGICL; | (SEQ ID NO: 4) |
| Peptide 927-941: | |
| PAREIPDLLEKGERL; | (SEQ ID NO: 5) |
| and | |
| Peptide 1166-1180: | |
| TLERPKILSPGKNGV. | (SEQ ID NO: 6) |

In embodiments where donors have HLA A2.1 blood type (HLA-A2pos) HER2 MHC class I peptides or epitopes comprise:

| | | |
|---|---|---|
| Peptide 369-377: | KIFGSLAFL; | (SEQ ID NO: 7) |
| and | | |
| Peptide 689-697: | RLLQETELV. | (SEQ ID NO: 8) |

“HER2^{pos}” is the classification or molecular subtype of a type of breast cancer as well as numerous other types of cancer. HER2 positivity is currently defined by gene amplification by FISH (fluorescent in situ hybridization) assay and 2+ or 3+ on intensity of immunohistochemical staining.

“HER2^{neg}” is defined by the lack of gene amplification by FISH, and can encompass a range of immunohistochemical staining from 0 to 2+ in most cases.

“Isolated” means separated or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

“LPS” means lipopolysaccharide, a bacterial cell wall component recognized by dendritic cells through Toll-like receptor 4.

“LTA” is defined as lipoteichoic acid which is a bacterial cell wall component recognized by dendritic cells through Toll-like receptor 2.

The term “major histocompatibility complex” or “MHC” as used herein is defined as a specific cluster of genes, many of which encode evolutionary related surface proteins involved in antigen presentation, which are among the most important determinants of histocompatibility. Class I MHC, or MHC class I, function mainly in antigen presentation to CD8 T lymphocytes. Class II MHC, or MHC class II, function mainly in antigen presentation to CD4⁺ T lymphocytes (T-helper cells).

“Mature DC” as used herein means a dendritic cell that expresses molecules, including high levels of MHC class II, CD80 (B7.1) and CD86 (137.2) molecules. In contrast, immature DCs (“iDCs” or “IDCs”) express low levels of MHC class II, CD80 (B7.1) and CD86 (B7.2) molecules, yet can still take up an antigen. “Mature DC” also refers to an antigen presenting cell existing in vivo, in vitro, ex vivo, or in a host or subject that may also be DC1-polarized (i.e., fully capable of promoting cell-mediated immunity.)

“Metrics” of CD4⁺ Th1 responses (or Th1 responses) are defined for each subject group analyzed for anti-HER2 CD4⁺ Th1 immune response: (a) overall anti-HER2 responsivity (expressed as percent of subjects responding to ≧1 reactive peptide); (b) response repertoire (expressed as mean number of reactive peptides (n) recognized by each subject group); and (c) cumulative response (expressed as total sum of reactive spots (spot-forming cells “SFC” per 10 cells from IFN-γ ELISPOT analysis) from 6 MHC Class II binding peptides from each subject group.

“Non-equivocal HER2^{neg} is defined as non-gene amplified and 0 or 1+ on immunohistochemical staining. “Equivocal HER2^{neg}” is defined as non-gene amplified but 2+ on immunohistochemical staining.

“Responsivity” or “anti-HER2 responsivity” are used interchangeably herein to mean the percentage of subjects responding to at least 1 of 6 binding peptides.

“Response repertoire” is defined as the mean number (“n”) of reactive peptides recognized by each subject group.

“Sample” or “biological sample” as used herein means a biological material from a subject, including but is not limited to blood, organ, tissue, exosome, plasma, saliva, urine and other body fluid. A sample can be any source of material obtained from a subject.

“Signal 1” as used herein generally refers to the first biochemical signal passed from an activated DC to a T cell. Signal 1 is provided by an antigen expressed at the surface of the DC and is sensed by the T cell through the T cell receptor.

“Signal 2” as used herein generally refers to the second signal provided by DCs to T cells. Signal 2 is provided by “costimulatory” molecules on the activated DC, usually CD80 and/or CD86 (although there are other costimulatory molecules known), and is sensed by the T cell through the surface receptor CD28.

“Signal 3” as used herein generally refers to the signal generated from soluble proteins (usually cytokines) produced by the activated DC. These are sensed through receptors on the T lymphocyte. The 3^{rd} signal instructs the T cell as to which phenotypical or functional features they should acquire to best deal with the current threat.

The terms “subject,” “patient,” “individual,” and the like are used interchangeably herein, and refer to any animal, or cells thereof whether in vitro or in situ, amenable to the methods described herein. In certain non-limiting embodiments, the patient, subject or individual is a human.

The term “targeted therapies” as used herein refers to cancer treatments that use drugs or other substances that interfere with specific target molecules involved in cancer cell growth usually while doing little damage to normal cells to achieve an anti-tumor effect. Traditional cytotoxic chemotherapy drugs, by contrast, act against all actively dividing cells. In breast cancer treatment monoclonal antibodies, specifically trastuzumab/Herceptin® targets the HER2/neu receptor.

“T/C” is defined as trastuzumab and chemotherapy. This refers to patients that receive both trastuzumab and chemotherapy before/after surgery for breast cancer.

The terms “T-cell” or “T cell” as used herein are defined as a thymus-derived cell that participates in a variety of cell-mediated immune reactions.

The terms “T-helper cells,” “helper T cells,” “Th cells,” and the like are used herein with reference to cells indicates a sub-group of lymphocytes (a type of white blood cell or leukocyte) including different cell types identifiable by a skilled person in the art. In particular, T-helper cells are effector T-cells whose primary function is to promote the activation and functions of other B and T lymphocytes and/or macrophages. Helper T cells differentiate into two or more major subtypes of cells including “Th1” or “Type 1” and “Th2” or “Type 2” phenotypes. These Th cells secrete cytokines, proteins, or peptides that stimulate or interact with other leukocytes. “Th1 cell,” “CD4⁺ Th1 cell,” “CD4⁺ T-helper type1 cell,” “CD4⁺ T cell” and the like as used herein refer to a mature T-cell that has expressed the surface glycoprotein CD4. CD4⁺ T-helper cells become activated when they are presented with peptide antigens by MHC class II molecules which are expressed on the surface of antigen-presenting peptides (“APCs”) such as dendritic cells. Upon activation of a CD4⁺ T helper cell by the MHC-antigen complex, it secretes high levels of cytokines such as interferon-γ (“IFN-γ”). Such cells are thought to be highly effective against certain disease-causing microbes that live inside host cells, and are critical in antitumor response in human cancer.

“Th17 T cell” as used herein refers to a T cell that produces high levels of the cytokines IL-17 and IL-22 and is thought to be highly effective against disease-causing microbes that live on mucousal surfaces.

“Treg” “T_{reg}” and “regulatory T-cells” are used herein to refer to cells which are the policemen of the immune system, and which act to regulate the anti-cancer activities of the immune system. They are increased in some cancers, and are mediators in resistance to immunotherapy in these cancer types.

“Therapeutically effective amount” or “effective amount” are used interchangeably herein, and refer to an amount of a compound, formulation, material, or composition, as described herein, that when administered to a patient, is effective to achieve a particular biological result. The amount of a compound, formulation, material, or composition described herein, which constitutes a “therapeutically effective amount” will vary depending on the compound, formulation, material, or composition, the disease state and its severity, the age of the patient to be treated, and the like. The therapeutically effective amount can be determined routinely by one of ordinary skill in the art having regard to his/her own knowledge and to this disclosure.

The term “Toll like receptor” or “TLR” as used herein is defined as a class of proteins that play a role in the innate immune system. TLRs are single membrane-spanning, non-catalytic receptors that recognize structurally conserved molecules derived from microbes. TLRs activate immune cell responses upon binding to a ligand.

The terms “treat,” “treating,” and “treatment,” refer to therapeutic or preventative measures described herein. The methods of“treatment” employ administration to a subject, in need of such treatment, a composition or method of the present embodiments, for example, a subject afflicted with a disease or disorder, or a subject who ultimately may acquire such a disease or disorder, in order to prevent, cure, delay, reduce the severity of, or ameliorate one or more symptoms of the disorder or recurring disorder, or in order to prolong the survival of a subject beyond that expected in the absence of such treatment.

The term “vaccine” as used herein is defined as a material used to provoke an immune response after administration of the material to an animal, preferably a mammal, and more preferably a human. Upon introduction into a subject, the vaccine is able to provoke an immune response including, but not limited to, the production of antibodies, cytokines and/or other cellular responses.

Ranges: throughout this disclosure, various aspects of the embodiments can be presented in a range format. It should be understood that the description in range format is merely for convenience and brevity and should not be construed as an inflexible limitation on the scope of the embodiments. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Reference will now be made in detail to several embodiments, examples of which are also illustrated in the accompanying drawings, photographs, and/or illustrations.

### DESCRIPTION

The lifetime risk of breast cancer development is nearly one in eight. The erb-B2 oncogene (HER-2/neu) is a molecular driver that is overexpressed in a significant number of breast, ovarian, gastric esophageal, lung, pancreatic, prostate and other solid tumors. HER2 overexpression (“HER2^{pos}”), a molecular oncodriver in several tumor types including approximately 20-25% of breast cancers, is associated with a more clinically aggressive disease, resistance to chemotherapy, higher rates of recurrence and metastasis, and worse overall prognosis. In incipient breast cancer, HER2 overexpression is associated with enhanced invasiveness, tumor cell migration, and the expression of proangiogenic factors, suggesting a critical role for HER2 in promoting a tumorigenic environment. In a retrospective analysis of ductal carcinoma in situ (“DCIS”) patients, DCIS lesions overexpressing HER2 were over six times as likely to be associated with invasive breast cancer than were DCIS lesions without HER2 overexpression.

Although molecular targeting therapies targeting HER2, i.e., trastuzumab, has resulted in tremendous positive clinical effect in this type of breast cancer, the almost universal resistance to the existing HER2 therapies in advanced disease states, plus disease relapse in a sizeable proportion of women who receive the targeted therapy prove the need for additional strategies targeting HER2. The promise of vaccines that activate the immune system against HER2 which seek to mitigate tumor progression and preventing recurrence while encouraging, is yet to be fully realized. Therefore there remains a need for additional tests and therapies to diagnose and treat HER2 breast cancer. The present embodiments described herein address these issues.

The role of systemic anti-HER2 CD4⁺ Th1 responses in HER2-driven breast tumorigenesis, however, remains unclear. The embodiments described herein are based on the identification of a progressive loss of anti-HER2 CD4⁺ Th1 response across a tumorigenic continuum in HER2-breast cancer, which appears to be HER2-specific and regulatory T-cell (T_{reg})-independent. Specifically, there is an inverse correlation of anti-HER2 CD4⁺ Th1 responses with HER2 expression and disease progression. Additionally, the depressed anti-HER2 Th1 responses in HER2^{pos}-invasive breast cancer were differentially restored after HER2-pulsed type-1 polarized dendritic cell (“DC1”) vaccinations, but the depressed responses were not restored following HER2-targeted therapy with trastuzumab and chemotherapy (“T/C”) as will be detailed herein or by other standard therapies such as surgical resection or radiation. The restored anti-HER2 Th1 responses also appear to be durable for at least about six months or longer.

Preferred embodiments described herein provide materials and methods for generating, and detecting the circulating anti-cancer CD4⁺ Th1 response in mammalian subjects. Blood tests/assays are provided which generate a circulating anti-cancer CD4⁺ Th1 response (i.e., IFN-γ-secreting) and the resulting IFN-γ production is detected and measured. In other preferred embodiments, subject blood samples containing CD4⁺ Th1 cells and antigen-presenting cells or precursors thereof are pulsed with MHC class II immunogenic peptides based on the type of cancer the subject is afflicted with and which are capable of inducing an immune response in said subject. Preferably the antigen-presenting cells or precursors thereof are mature or immature dendritic cells or monocyte precursors thereof. In particularly preferred embodiments, the cancer is preferably HER2-expressing and the mammalian subject is preferably a human, and more preferably the cancer is HER2^{pos} breast cancer and the human subject is a female.

The herein identified anti-HER2 CD4⁺ Th1 response decrement allows the detected immune response generated in such blood tests to be used as a cancer diagnostic/response predictor alone or in tandem with the use of specialized vaccines to restore a patient's immune response. The preferred embodiments described herein thus shift the focus of cancer diagnosis and therapy to patient immunity and use of blood tests to determine and/or predict the immune response against a cancer, including patients at risk for recurrence, as opposed to diagnosis and treatment methods that rely on identification of tumor cells.

A preferred embodiment is provided for generating a circulating anti-HER2 CD4⁺ Th1 response in a mammalian subject by isolating unexpanded peripheral blood mononuclear cells (“PBMCs”) from a subject and pulsing the PBMCs with a composition comprising HER2-derived MHC class II antigenic binding peptides capable of generating an immune response in the subject. Without wishing to be bound by any particular theory, when the binding peptides are presented to CD4⁺ Th1 cells that are present in the PBMC sample they activate the CD4⁺ Th1 cells and the activated CD4⁺ Th1 cells produce interferon-γ (“IFN-γ”). DC s (type-1 polarized dendritic cells) derived from precursor pluripotent monocytes contained in the subject's PBMC sample are antigen-presenting cells (“APCs”) which upon exposure to the binding peptides become antigen-loaded APCs which present the MHC class II antigen binding peptides to the subject's CD4⁺ Th1 cells in the sample thereby activating the CD4⁺ Th1 cells to produce/secrete IFN-γ. The IFN-γ thereby produced is subsequently measured for analysis.

In an alternate preferred embodiment, a circulating anti HER2 CD4⁺ Th1 response is generated in a mammalian subject by co-culturing previously unstimulated purified CD4⁺ T-cells from a subject blood sample with autologous immature or mature dendritic cells (“iDCs” or “mature DCs”, collectively, “DCs”) pulsed with a composition comprising HER2-derived MHC class I antigenic binding peptides capable of generating an immune response in the subject. Without wishing to be bound by any particular theory, when the binding peptides are presented to CD4⁺ Th1 cells present in the T-cell sample they activate the CD4⁺ Th1 cells and the activated CD4⁺ Th1 cells produce/secrete IFN-γ. The immature DCs are matured to DC1's, which present the MHC class II binding peptides to the subject's CD4⁺ Th1 cells that are present in the sample thereby activating the CD4⁺ Th1 cells to produce IFN-γ, which is subsequently measured for analysis.

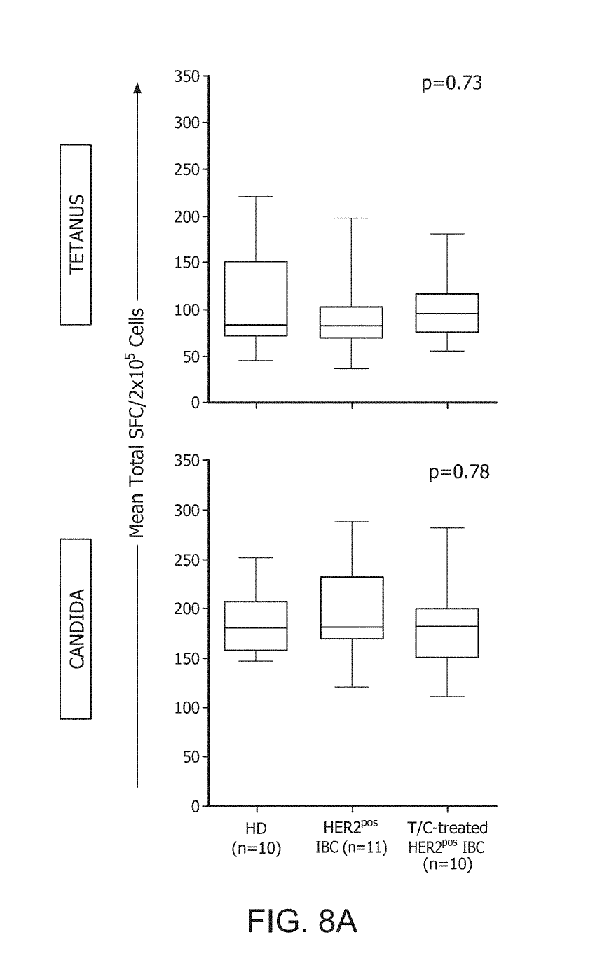

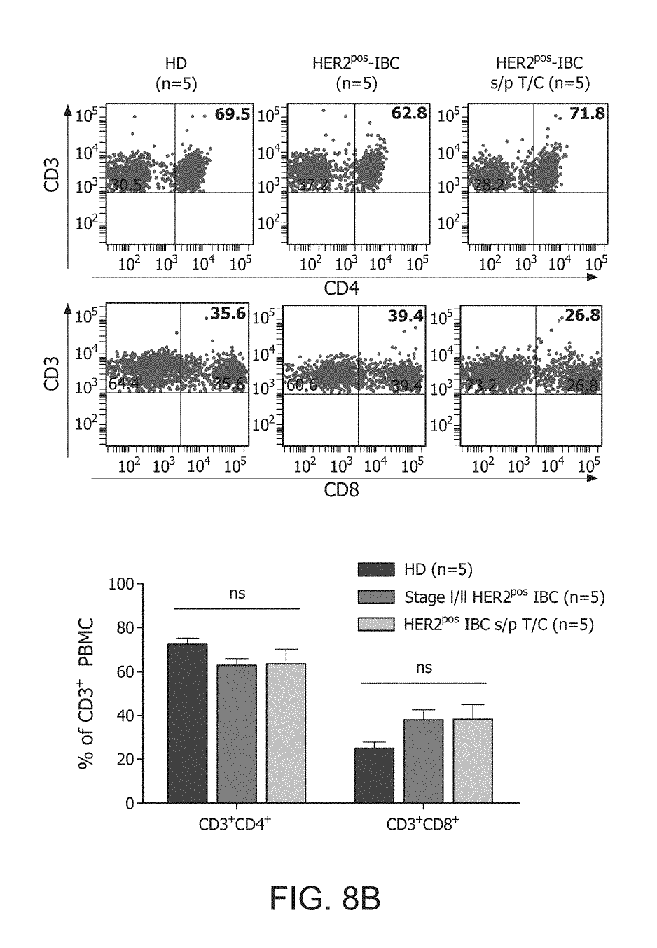

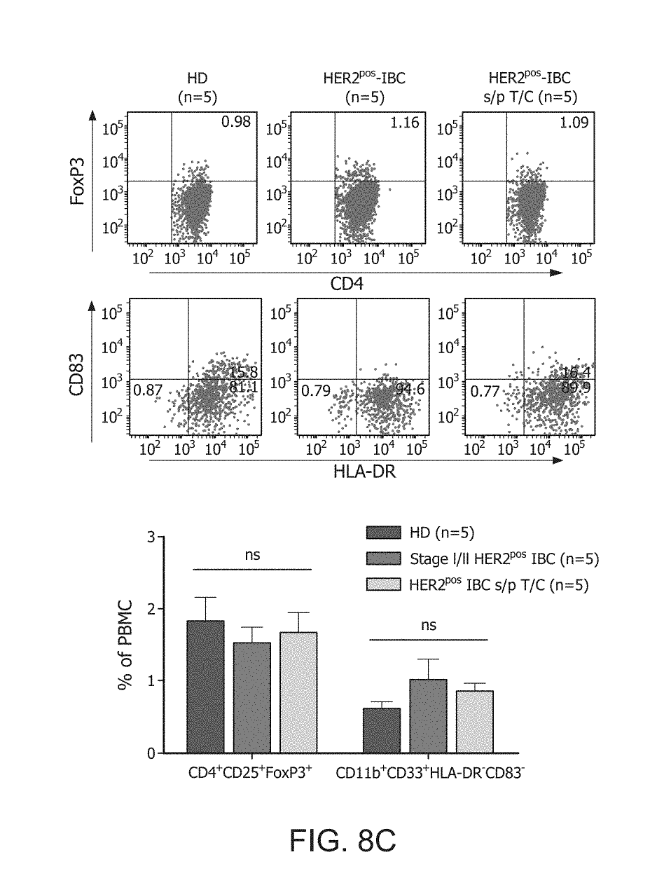

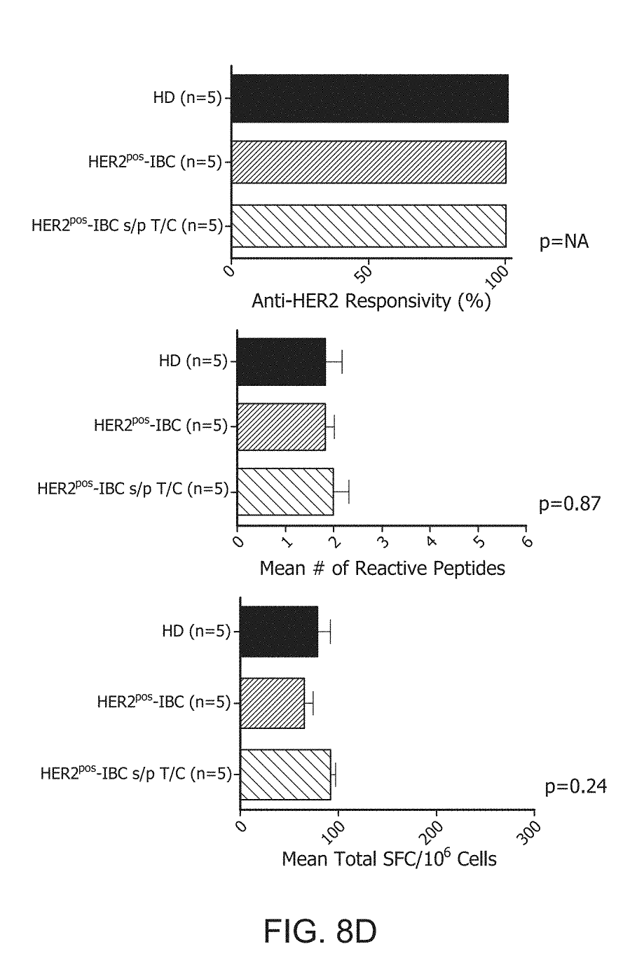

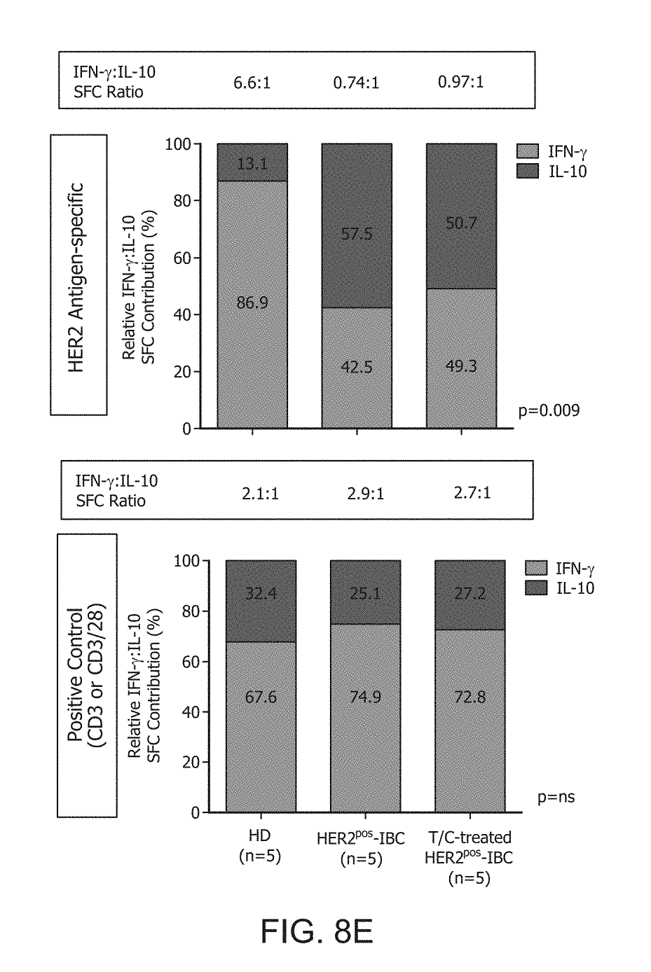

In both alternate preferred embodiments for generating anti-HER2 immune response in a subject, IFN-γ produced by anti-HER2 CD4⁺ Th1 cells is detected and measured via IFN-γ enzyme-linked immunospot (“ELISPOT”) assay, although it should be understood by one skilled in the art that other detection methods may be used. For example, flow cytometry, enzyme-linked immunosorbent assay (“ELISA”), and immunofluorescence (“IF”) can be used for monitoring immune response. Alternatively, in instances of immune monitoring of patients, it can be advantageous to measure the ratio of IFN-γ to IL-10 (as was done in the Reference Example and shown in FIG. 8E) as opposed to, or in addition to, a straight IFN-γ test such as ELISPOT which shows total CD4⁺ cell spots. Such testing would be particularly advantageous for patients at risk. Further, the use of immunofluorescence provides other ways to measure and visualize immune response via use of ELISPOT readers that read results by fluorescence. In such instances the results can be arranged to show 2, 3, or more cytokines/other secreted immune molecules, each showed in a different color, in the same patient sample.

Those skilled in the art can readily appreciate, other suitable APC's may be used in addition to dendritic cells and monocytes, such as, for example, macrophages, and B cells.

In preferred embodiments IFN-γ ELISPOT assays are performed to detect IFG-γ production (positive peptide response: threshold minimum 20 SFC/2×10 and 2× greater than unstimulated control). Results are preferably expressed as three metrics of Th1 response: (a) overall anti-HER2 responsivity (expressed as percent of subjects responding to ≧1 reactive peptide); (b) response repertoire (expressed as mean number of reactive peptides (n) recognized by each subject group); and (c) cumulative response (expressed as total sum of reactive spots (spot-forming cells “SFC” per 10⁶ cells from IFN-γ ELISPOT analysis) from all 6 MHC class II binding peptides from each subject group.

In preferred embodiments for HER2^{pos} cancers, DCs, immature or type-1 polarized DC1s, are pulsed with a composition comprising 6 MHC class II binding peptides or epitopes derived from or based on HER2 that are capable of generating an immune response in a patient and optionally pulsed with 2 MHC class I binding peptides or epitopes when patients have an HLA A2.1 blood type. HER2 MHC class II binding peptides or epitopes include:

| | |
|---|---|
| Peptide 42-56: | |
| HLDMLRHLYQGCQVV; | (SEQ ID NO: 1) |
| Peptide 98-114: | |
| RLRIVRGTQLFEDNYAL; | (SEQ ID NO: 2) |
| Peptide 328-345: | |
| TQRCEKCSKPCARVCYGL; | (SEQ ID NO: 3) |
| Peptide 776-790: | |
| GVGSPYVSRLLGICL; | (SEQ ID NO: 4) |
| Peptide 927-941: | |
| PAREIPDLLEKGERL; | (SEQ ID NO: 5) |
| and | |
| Peptide 1166-1180: | |
| TLERPKTLSPGKNGV. | (SEQ ID NO: 6) |

In embodiments where donors have A2.1 blood type HER2 MHC class I peptides or epitopes include:

| | | |
|---|---|---|
| Peptide 369-377: | KIFGSLAFL; | (SEQ ID NO: 7) |
| and | | |
| Peptide 689-697: | RLLQETELV. | (SEQ ID NO: 8) |

As described further herein, the HER2 binding peptides/epitopes of the preferred embodiments are not limited to the six above-referenced peptides and also include peptides that are functional equivalents or alternatives of the binding peptides identified by SEQ ID NOS: 1-6 as will be discussed in more detail herein. There are additional class I peptides that may be used for subjects with A2.1 and A3.1 blood types as well as other blood types (e.g., A5, A6) which comprise class I peptides that bind any phenotype.

There are many other HER2^{pos} solid cancers in addition to breast cancer, such as, for example, brain, bladder, esophagus, lung, pancreas, liver, prostate, ovarian, colorectal, and gastric, and others, for which the materials and methods of the embodiments described herein can be used for diagnosis and treatment. Therefore, the six anti-HER2 binding peptides described above may be used in accordance with the herein embodiments to generate immune responses capable of detection and useful for diagnostics for these and other HER2-expressing cancers.

Vaccines can be developed to target HER2-expressing tumors using the same anti-HER2 binding peptides described above or may employ any composition of HER2 that is immunogenic such as, for example, DNA, RNA, peptides, or proteins or components thereof such as the ICD and ECD domains. For example, subjects can be vaccinated against the whole HER2 protein and the six above-referenced binding peptides can be used to monitor the patient's immune response. Similarly, vaccines can be developed for other types of cancer such as other members of the HER2 family which includes HER1, HER3, and c-MET.

Although the present preferred embodiments are directed to treating and diagnosing HER2^{pos} breast cancer in women it should be readily appreciated by the skilled artisan that the present embodiments are not limited to female humans. The presently preferred embodiments include male humans, for example, HER2-expressing prostate cancer, as well as other mammalian subjects

### Compositions

The preferred embodiments include use of isolated peptides derived from or otherwise based on the HER2 protein. The binding peptides of the preferred embodiments represent epitopes of the corresponding HER2 protein. Although a presently preferred embodiment features six HER2 MHC class II binding peptides/epitopes, other possible MHC class II HER2 peptides can be used in the present embodiments in that any components of the entire HER2 molecule can be used as a source for other binding peptides so long as they are sufficiently immunologically active in patients.

In preferred embodiments, the HER2 binding peptides comprise six HER2 MHC class II binding peptides, having the sequences:

| | |
|---|---|
| Peptide 42-56: | |
| HLDMLRHLYQGCQVV; | (SEQ ID NO: 1) |
| Peptide 98-114: | |
| RLRIVRGTQLFEDNYAL; | (SEQ ID NO: 2) |
| Peptide 328-345: | |
| TQRCEKCSKPCARVCYGL; | (SEQ ID NO: 3) |
| Peptide 776-790: | |
| GVGSPYVSRLLGICL; | (SEQ ID NO: 4) |
| Peptide 927-941: | |
| PAREIPDLLEKGERL; | (SEQ ID NO: 5) |
| and | |
| Peptide 1166-1180: | |
| TLERPKTLSPGKNGV. | (SEQ ID NO: 6) |

The HER2 epitope identified by SEQ ID NO: 1 represents positions 42-56 of the HER2 protein. The HER2 epitope identified by SEQ ID NO: 2 represents positions 98-114 of the HER2 protein. The HER2 epitope identified by SEQ ID NO: 3 represents positions 328-345 of the HER2 protein. The HER2 epitope identified by SEQ ID NO: 4 represents positions 776-790 of the HER2 protein. The HER2 epitope identified by SEQ ID NO: 5 represents positions 927-941 of the HER2 protein. The HER2 epitope identified by SEQ ID NO: 6 represents positions 1166-1180 of the HER2 protein.

Further, the skilled artisan can further appreciate that embodiments described herein are not limited to the use of all 6 of the binding peptides described in connection with preferred embodiments herein. Any number of the described binding peptides may be employed in patient blood tests, with the lower range being about two or three, with the caveat that there must be sufficient immunological activity with the patient's CD4⁺ t-cells so as to cause production of IFN-γ. Therefore, in instances where HER-derived Class II biding peptides are used which are fewer than/different than those of the set of six described in connection with the preferred embodiments herein, the number of binding peptides may well be substantially less than or greater than six depending on the immune responses generated in subjects.

As described herein, the HER2 binding peptides of the preferred embodiments also encompass peptides that are functional equivalents of the peptides identified by SEQ ID NOS: 1-6. Such functional equivalents may have an altered sequence in which one or more of the amino acids in the corresponding HER2 peptide sequence are substituted or in which one or more amino acids are deleted from or added to the corresponding reference sequence. For example, 1 to 3 amino acids may be added to the amino terminus, carboxy terminus, or both. In some examples, the HER2 peptides can be glycosylated.

The HER2 binding peptides or any peptide in accordance with the present embodiments may be cyclized or linear. When cyclized, the epitopes may be cyclized in any suitable manner. For example, disulfide bonds may be formed between selected cysteine (“Cys”) pairs in order to provide a desired confirmation. It is believed that the formation of cyclized epitopes may provide conformations that improve the immune response.

In other instances, the HER2 binding peptides may be the retro-inverso isomers of the HER2 binding peptides. The retro-inverso modification comprises the reversal of all amide bonds within the peptide backbone. This reversal may be achieved by reversing the direction of the sequence and inverting the chirality of each amino acid residue by using D-amino acids instead of the L-amino acids. This retro-inverso isomer form may retain planarity and conformation restriction of at least some of the peptide bonds.

Non-conservative amino acid substitutions and/or conservative substitutions may also be made. Substitutions are conservative amino acid substitutions when the substituted amino acid has similar structural or chemical properties with the corresponding amino acid in the reference sequence. By way of example, conservative amino acid substitutions involve substitution of one aliphatic or hydrophobic amino acid, e.g., alanine, valine, leucine and isoleucine, with another; substitution of one hydroxyl-containing amino acid, e.g., serine and threonine, with another, substitution of one acidic residue, e.g., glutamic acid or aspartic acid, with another, replacement of one amide-containing residue, e.g., asparagine and glutamine, with another, replacement of one aromatic residue, e.g., phenylalanine and tyrosine, with another, replacement of one basic residue, e.g., lysine, arginine and histidine, with another, and replacement of one small amino acid, e.g., alanine, serine, threonine, methionine, and glycine, with another.

In some instances, the deletions and additions are located at the amino terminus, the carboxy terminus, or both, of one of the sequences of the binding peptides of the preferred embodiments. For example, a HER2 binding peptide equivalent has an amino acid sequence which is at least 70% identical, at least 80% identical, at least 85% identical, at least 90% identical, at least 91%, at least 92%, at least 93%, at least 94%, at least 95%, at least 96%, at least 97%, at least 98%, or at least 99% identical to the corresponding HER2 binding peptide sequences. Sequences which are at least 90% identical have no more than 1 alteration, i.e., any combination of deletions, additions or substitutions, per 10 amino acids of the reference sequence. Percent identity is determined by comparing the amino acid sequence of the variant with the reference sequence using known or to be developed programs in the art.

For functional equivalents that are longer than a corresponding HER2 binding peptide sequence, the functional equivalent may have a sequence which is at least 90% identical to the HER2 peptide sequence and the sequences which flank the HER2 peptide sequences in the wild-type HER2 protein.

Functional equivalents of the HER2 binding peptides may be identified by modifying the sequence of the peptide and then assaying the resulting polypeptide for the ability to stimulate a subject's monocytes, DC's or other antigen-presenting cells that present the binding peptides/epitopes to CD4⁺ Th1 cells.

In accordance with other embodiments, chimeric peptides and compositions comprising one or more chimeric peptides are provided. According to various embodiments, the chimeric peptides comprise a HER2 peptide, another peptide, and a linker joining the HER2 peptide to the other peptide. It will be further understood that any suitable linker may be used. For example, depending upon the peptide used, the HER2 binding peptide may be linked to either the amino or the carboxy terminus of the other binding peptide. The location and selection of the other peptide depends on the structural characteristics of the HER2 peptide, whether alpha helical or beta-turn or strand.

In another embodiment, the linker may be a peptide of from about 2 to about 15 amino acids, about 2 to about 10 amino acids, or from about 2 to about 6 amino acids in length. The chimeric peptides may be linear or cyclized. Additionally, the HER2 peptides, the other peptides, and/or the linker may be in retro-inverso form. Thus the HER2 peptide along could be in retro inverso form. Alternatively, the HER2 peptide and the other peptide could be in retro inverso form. In another example, the HER2 peptide, the other epitope, and the linker could be in retro inverso form.

Peptides, including chimeric peptides can be prepared using well known techniques. For example, the peptides can be prepared synthetically, using either recombinant DNA technology or chemical synthesis. Peptides of the present embodiments may be synthesized individually or as longer polypeptides composed of two or more peptides. The peptides of the presently preferred embodiments are preferably isolated, i.e., substantially free of other naturally occurring host cell proteins and fragments thereof.

The peptides and chimeric peptides of the present embodiments may be synthesized using commercially available peptide synthesizers. For example, the chemical methods described in Kaumaya, P. T. P., et al., “De Novo” Engineering of Peptide Immungenic and Antigenic Determinants as Potential Vaccines, in Peptides, Design. Synthesis and Biological Activity, pp 133-164 (1994), may be used. For example, HER2 binding peptides may be synthesized co-linearly with another binding peptide to form a chimeric peptide. Peptide synthesis may be performed using Fmoc/t-But chemistry. The peptides and chimeric peptides may be cyclized in any suitable manner. For example, disulfide bonds may be achieved using differentially protected cysteine residues, iodine oxidation, the addition of water to boost removal of Acm group and the concomitant formation of a disulfide bond, and/or the silyl chloride-sulfoxide method.

The peptides and chimeric peptides may also be produced using cell-free translation systems and RNA molecules derived from DNA constructs that encode the epitope or binding peptide. Alternatively, the epitopes or chimeric peptides may be made by transfecting host cells with expression vectors that comprise a DNA sequence that encodes the respective epitope or chimeric peptide and then inducing expression of the polypeptide in the host cells. For recombinant production, recombinant constructs comprising one or more of the sequences which encode the binding peptide epitope, chimeric peptide, or a variant thereof are introduced into host cells by conventional methods such as calcium phosphate transfection, DEAE-dextran mediated transfection, microinjection, cationic lipid-mediated transfection, electroporation, transduction, scrape lading, ballistic introduction or infection.

The binding peptides of the present embodiments may contain modifications, such as glycosylation, side chain oxidation, or phosphorylation; so long as the modifications do not destroy the biological activity of the binding peptides. Other modifications include incorporation of D-amino acids or other amino acid mimetics.

The binding peptides of the embodiments can be prepared as a combination, which includes two or more peptides. The peptides may be in a cocktail or may be conjugated to each other using standard techniques. For example, the peptides can be expressed as a single polypeptide sequence. The peptides in the combination may be the same or different.

The present embodiments should also be construed to encompass “mutants,” “derivatives,” and “variants” of the peptides of the embodiments (or of the DNA encoding the same) which mutants, derivatives and variants are peptides which are altered in one or more amino acids (or, when referring to the nucleotide sequence encoding the same, are altered in one or more base pairs) such that the resulting peptide (or DNA) is not identical to the sequences recited herein, but has the same biological property as the peptides disclosed herein.

Some embodiments also provide a polynucleotide encoding at least one peptide selected from a peptide having the sequence of any one or more of SEQ ID NOS: 1-6. The nucleic acid sequences include both the DNA sequence that is transcribed into RNA and the RNA sequence that is translated into a peptide. According to other embodiments, the polynucleotides are inferred from the amino acid sequence of the peptides of the preferred embodiments. As is known in the art several alternative polynucleotides are possible due to redundant codons, while retaining the biological activity of the translated peptides.

Further, preferred embodiments encompass an isolated nucleic acid encoding a peptide having substantial homology to the binding peptides disclosed herein. Preferably, the nucleotide sequence of an isolated nucleic acid encoding a peptide of the invention is “substantially homologous”, that is, is about 60% homologous, more preferably about 70% homologous, even more preferably about 80% homologous, more preferably about 90% homologous, even more preferably, about 95% homologous, and even more preferably about 99% homologous to a nucleotide sequence of an isolated nucleic acid encoding a binding peptide of preferred embodiments.

It is to be understood explicitly that the scope of the preferred embodiments encompasses homologs, analogs, variants, derivatives and salts, including shorter and longer peptides and polynucleotides, as well as peptide and polynucleotide analogs with one or more amino acid or nucleic acid substitution, as well as amino acid or nucleic acid derivatives, non-natural amino or nucleic acids and synthetic amino or nucleic acids as are known in the art, with the stipulation that these modifications must preserve the immunological activity of the original binding peptide. Specifically, any active fragments of the active binding peptides as well as extensions, conjugates and mixtures are encompassed according to the principles described herein.

The preferred embodiments should be construed to include any and all isolated nucleic acids which are homologous to the nucleic acids described and referenced herein, provided these homologous DNAs have the biological activity of the binding peptides disclosed herein.

The skilled artisan will understand that the nucleic acids of the preferred embodiments encompass an RNA or a DNA sequence encoding a peptide of a preferred embodiment, and any modified forms thereof, including chemical modifications of the DNA or RNA which render the nucleotide sequence more stable when it is cell free or when it is associated with a cell. Chemical modifications of nucleotides may also be used to enhance the efficiency with which a nucleotide sequence is taken up by a cell or the efficiency with which it is expressed in a cell. Any and all combinations of modifications of the nucleotide sequences are contemplated in the preferred embodiments.

Further, any number of procedures may be used for the generation of mutant, derivative or variant forms of a peptide of the preferred embodiments using recombinant DNA methodology well known in the art such as, for example, that described in Sambrook and Russell, supra, and Ausubel et al., supra. Procedures for the introduction of amino acid changes in a peptide or polypeptide by altering the DNA sequence encoding the polypeptide are well known in the art and are also described in these, and other, treatises.

The nucleic acids encoding the binding peptides of the preferred embodiments can be incorporated into suitable vectors e.g., retroviral vectors. These vectors are well known in the art. The nucleic acids or the vectors containing them usefully can be transferred into a desired cell, which cell is preferably from a patient.

### Cryopreservation

After PBMCs are obtained from subjects and separated, they can be cryopreserved before performing any blood tests/assays described herein using methods well known to the skilled artisan and as described further herein below.

### Use as Diagnostic/Prognostic/Treatment Monitoring Tool

As described herein, it has been found that loss of circulating HER2-reactive IFN-γ^{pos} CD4⁺ Th1 cells begins as early as DCIS breast cancer and substantially declines in early invasive Stage I HER2^{pos} tumors. More specifically, there is a stepwise anti-HER2 CD4⁺ Th1 response decrement across the continuum in breast tumorigenesis from healthy donors to HER2^{pos} DCIS (ductal carcinoma in situ) to HER2^{pos} IBC (invasive breast cancer). There are reasons for what this loss of immune response is not due to, namely, it is not due to cancer-related global immunosuppression, it is not likely related to an increase in translocation to invasive lesions, and it is independent of regulatory T cells.

An embodiment based on this finding of loss of anti-HER2 CD4⁺ Th1 immune response provides a method for screening apparently healthy individuals for breast and other cancers that might not be detected via mammography or other screening approaches, comprising performing rapid immune tests/assays, the blood tests of the preferred embodiments, for detecting anti-HER2 CD4⁺ Th1 responsiveness. Test results for such individuals that are lower than those for healthy individuals, would allow for more definitive testing and quicker exercising of therapeutic options. For example, the blood tests herein can be advantageously used to identify patients at risk in whom vaccination may be considered to reduce risk of HER-2 expressing breast cancer. For instance, patients at risk may be those following completion of lactation, pregnancies, and other life stressing events that may reduce the response.

Such a screening method can also be beneficial for patients at high risk for developing breast cancer, due to factors such as genetic disposition or lifestyle factors. From a diagnostic perspective, an immune biomarker can be developed to screen such high-risk patients for fluctuations in their anti-HER2 Th1 immunity. While IHC staining or FISH profiling of breast biopsy specimens offer only an isolated snapshot of a tumor's evolution, immune profiling (such as with this potential biomarker) may provide a glimpse into the natural history and immune repercussions of a tumor. It can be used to predict patients diagnosed with any breast cancer whether they may be at risk for a HER-2 expressing new breast event or recurrence.

In another embodiment, diagnostic or monitoring tests based on loss of anti-HER2 Th1 response may be used to predict whether a patient with HER2^{pos} breast cancer will respond well to standard non-immune therapy such as chemotherapy plus trastuzumab.

According to a further embodiment, as will be detailed herein, CD4⁺ Th1 responses are capable of being preferentially restored via autologous DC1 vaccination with HER2-derived Class II peptides (DC1 immunization) as compared with targeted (e.g., trastuzumab) or conventional (i.e., chemotherapy) breast cancer therapies. As such, in HER2^{pos}-IBC patients, CD4+Th1 responses were effectively restored after HER2-pulsed DC vaccination, but not following trastuzumab/cytotoxic chemotherapy (“T/C”) treatment. The blood tests of the preferred embodiments are therefore performed pre-vaccination and post-vaccination to determine the extent of restoration, or non-restoration of Th1 immune response. Thus, a patient can have their CD4⁺ Th1 response easily reevaluated after breast cancer therapy via the blood tests herein to determine if any previously found CD4⁺ Th1 immune loss has been restored by vaccination.

In another embodiment, a blood test relying on the anti HER2 CD4⁺ Th1 response decrement may be used to determine whether DC vaccination has adequately restored or increased anti-HER2 immunity to levels capable of providing protection against further incursions of cancer. Post-DC1 vaccination, the blood tests of the preferred embodiments can be performed numerous times, preferably on a schedule as recommended by the patient's physician, so as to track the patient's CD4⁺ Th1 immune status. These additional tests may take place many months, e.g., at least up to about 60 months or more, after vaccination due to the durability of the vaccine-induced sensitization to the HER2 tumor target and the likeliness of protection over long periods of time.

Use of the blood tests herein may be used to show the degree of HER2-responsiveness post-chemo/trastuzumab treatment for HER2-expressing invasive breast cancer patients. A correlation is shown herein with how well such patients will respond to therapy, and thereby are predictive of outcome. For example, depressed anti-HER2 th1 responses predict an increased risk of subsequent recurrence in adjuvant-T/C-treated patients.

Other embodiments provide methods for an immune strategy, i.e., DC vaccination, to enhance or restore the anti-HER2 CD4⁺ Th1 loss found in HER2^{pos} invasive breast cancer patients. This capacity for “immunorestoration” can be exploited for therapy in combination with current trastuzumab regimens. It would also provide a rationale for combining vaccination with standard therapies including chemotherapy plus trastuzumab.

Another embodiment suggests an immune correlate for predicting risk of new breast events. In HER2^{pos}-IBC patients treated with chemotherapy/trastuzumab, it was shown that response variations, and more particularly, depressed anti-HER2 CD4⁺ Th1 responses, are associated with an increased risk of new breast cancer events. Thus such depressed responses can be used to predict outcomes as to whether a patient will likely endure a new breast event and if so, will likely require additional therapy. A biomarker can thus be developed based thereon.

Further, the observation described herein that HER2^{pos} breast cancer cells, expressing IFN-γ and TNF-α receptors, undergo apoptosis upon exposure to Th1-derived cytokines (including IFN-γ and TNF-α, the archetypical cytokines produced by Th1 cells) suggests that anti-HER2 Th1 cells may be instrumental in controlling or eliminating HER2-expressing cells during physiologic processes such as breast involution. IFN-γ and TNF-α receptor expression was found on all HER2^{pos} breast cancer cell lines tested as described herein, and it was seen that these anti-HER2 CD4⁺ Th1 cells produce soluble factors that cause apoptosis of HER2-expressing breast cancer cell lines. This suggests that anti-HER2 Th1 may be instrumental in controlling or eliminating HER2-expressing cells during physiologic processes such as breast involution and may explain how CD4⁺ Th1 cells, which cannot recognize Class II^{neg} Class I^{pos} tumor cells, can nonetheless mediate tumor cell destruction.

As described in detail herein, a further embodiment provides an immune correlate for predicting pathologic responsiveness to standard neoadjuvant therapy in HER2^{pos} breast cancer. Experiments were designed to study how the degree of HER2 responsiveness post-chemo/trastuzumab treatment for HER2-expressing invasive breast cancer patients correlates with how well they will respond to therapy. Such experiments revealed an immune correlate for predicting pathologic responsiveness to standard neoadjuvant therapy. An association was found between neoadjuvant complete responders and significantly higher anti-HER2 CD4⁺ Th1 responses, compared with patients who did not have pathologic complete responses.

While the magnitude of HER2-specific Th1 depression for T/C-treated HER2^{pos}-IBC patients correlates with an increased risk of subsequent recurrence of new breast events, in contrast, the above-described preservation of anti-HER2 CD4⁺ Th1 immunity is associated with complete pathologic response to neoadjuvant chemotherapy. Taken together, these data suggest that anti-HER2 Th1 immune reactivity may be used as a biomarker to help identify vulnerable patient subgroups at risk of clinical or pathologic failure.

Although the present embodiments as described herein may include specific reference to HER2-expressing breast tumors, it should be understood by those skilled in the art that other types of HER2-expressing tumors such as, for example only, ovarian, gastric esophageal, lung, pancreatic, liver, prostate and other solid tumors, may benefit from the teachings of the present embodiments. Similarly, those skilled in the art can appreciate that the teachings herein can extend to non-HER2-expressing breast cancer, including triple-negative and ER-positive as well as other tumors.

Additionally, there are other HER family targets from the receptor tyrosine kinase family that can be used in accordance with the preferred embodiments. The HER family consists of four related signaling molecules: HER1, HER2, HER3, and HER4 that are involved in a variety of cancers. While it is known that overexpression of HER-2 is found in about 20% to 25% of breast cancers, it has been found that other HER family members are involved in both early and invasive breast cancer, as well as other cancers. For example, HER1 is expressed on a small number of breast cancers, generally those that are triple negative. C-Met is a growth factor receptor involved in recurrence of many cancers that activates HER3. HER3 is overexpressed in colon, prostate, breast and melanoma. HER3 is expressed in a large number of DCIS lesions and breast cancers. HER3 can be detected in the residual DCIS at the time of surgery in some patients who received the DC HER2 vaccination. Thus, other HER family targets such as HER3, HER1 and c-Met that cause breast cancer and other solid cancers may be beneficially targeted and peptide vaccines against these other targets developed as was done for HER2 described herein. Accordingly, a breast cancer panel containing oncodrivers/proposed oncodrivers such as HER2, HER3, HER1 and C-Met for identifying which molecules are expressed in a patient's breast tumor can be developed as a therapy aid and used as vaccine target molecules. Thus it is contemplated that in addition to the DC1 vaccine described herein for HER2 similar vaccines can be developed for the non-HER2-expressing breast cancer types.

### EXAMPLES

The preferred embodiments are further described in detail by reference to the following experimental examples. These examples are provided for purposes of illustration only, and are not intended to be limiting unless otherwise specified. Thus, the preferred embodiments should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present embodiments and practice the claimed methods. The following working examples therefore, specifically point out the preferred embodiments, and are not to be construed as limiting in any way the remainder of the disclosure.

The following Reference Example includes Methods, Results, and Discussion sections.

### REFERENCE EXAMPLE

### Methods

### Patient Selection and Study Design

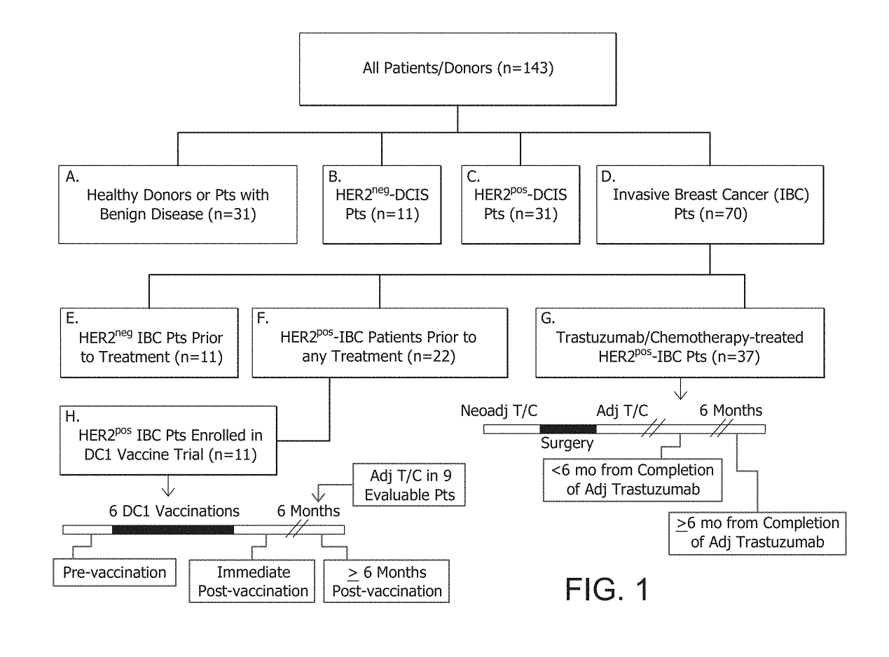

After approval by the Institutional Review Board of the University of Pennsylvania, 143 patients were consecutively recruited to participate in the presently described study and informed consent was obtained. Anti-HER2 CD4⁺ Th1 (“Th1”) responses were examined in healthy donors (“HD”) (n=21), and patients with benign breast disease (“BD”) (n=10), HER2^{neg}-DCIS (n=11), HER2^{neg} (0/1+) IBC (n=11), HER2^{pos}-DCIS (n=31), and HER2^{pos}-IBC (n=22) patients. Th1 responses of patients enrolled in neoadjuvant DC1 immunization trials for HER2^{pos}-DCIS and found to have Stage I HER2^{pos}-IBC at surgery (n=1), were analyzed pre- and post-immunization (immediately and ≧6 months after). Th1 responses in treatment-naïve HER2^{pos}-IBC patients were compared with responses in T/C-treated Stage I-III HER2^{pos}-IBC patients (n=37). FIG. 1 shows study-eligible patient and donor cohorts. T/C-treated HER2^{pos}-IBC patients were surveilled for development of BEs, defined as any locoregional or distant recurrence. Table 1 below shows the demographic and tumor-related characteristics of the present study populations (age, race, AJCC pathological stage, hormone receptor status, timing of chemotherapy, and time from completion of trastuzumab (when applicable) for individual patient subgroups) (“IBC”: invasive breast cancer; “DCIS”: ductal carcinoma in situ; “T/C”: trastuzumab/chemotherapy). Following T/C treatment, HER2^{pos}-IBC patients were observed for development of subsequent breast events (“BEs”), defined as any locoregional or distant recurrence. Th1 immune responses of all subjects were generated and analyzed prospectively.

### Vaccine Trial Design and Immunization Procedure

Two neoadjuvant trials of HER2-pulsed, type 1-polarized DC vaccination “DC1 vaccination” for patients with HER2^{pos}-DCIS were conducted. DC vaccines were prepared as described previously. See, Koski, G. K., et al., J. Immonother. 35(1): 54 (2012) (“Koski, et al.”); Sharma, A., et al., Cancer 118(17):4354 (2012) (“Sharma, et al.”); Fracol, M., et al., Ann. Surg. Oncol. 20(10):3233 (2013); Lee, M. K. 4th, et al., Expert Rev. 8(11):e 74698 (2013); Czerniecki, B. J., et al., Cancer Res. 67(4):1842 (2007); Czerniecki, B. J., et al., Cancer Res. 67(14):6531 (2007); and U.S. Published Application US 2013/0183343 A1.

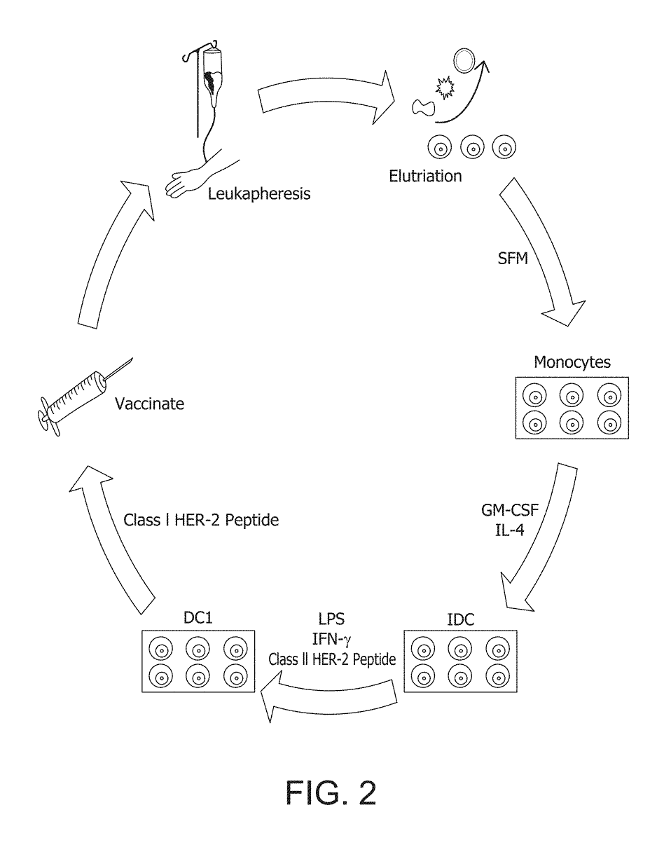

DC vaccination strategy used in the present studies is shown in FIG. 2. As shown therein, monocytic DC precursors (CD14⁺ peripheral blood monocytes) were obtained from subjects via tandem leukapheresis/countercurrent centrifugal elutriation. DCs were cultured overnight in macrophage serum-free medium (“SFM”) (Cellgro/Mediatech, Manassas, Va.) with granulocyte macrophage colony stimulating factor (“GM-CSF”) (250 IU/mL; Berlex, Wayne, N.J.) and IL-4 (1000 u/mL; R&D Systems, Minneapolis, Minn.)—these are considered immature DCs (“iDC”). The following day iDCs were pulsed with six HER2 MHC class II binding peptides (42-56 (SEQ ID NO: 1); 98-114 (SEQ ID NO: 2); and 328-345 (SEQ ID NO: 3) (extracellular domain of HER2), and 776-790 (SEQ ID NO: 4); 927-941 (SEQ ID NO: 5); and 1166-1180 (SEQ ID NO:6) (intracellular domain of HER2)) (see, Disis, M. L., et al., Clin. Can. Res. 5(6):1289 (1999) (“Disis, et al.”)) (United Biochemical Research, Seattle, Wash.; peptides stored lyophilized and reconstituted in sterile PBS for use). After 8-12 hours of incubation, IFN-γ (1,000 U/mL) was added. The following day, NIH reference standard lipopolysaccharide (“LPS”) was added (10 ng/mL) to achieve full DC activation to a type 1-polarized phenotype (“DC1”) 6 hours before harvest. For HLA-A2.1^{pos} patients, DC1s were pulsed with two additional MHC class I binding peptides (peptide 369-377 (SEQ ID NO: 7) and peptide 689-697 (SEQ ID NO: 8). Harvested cells were washed and lot release criteria of >70% viability, negative Gram stain, and endotoxin<5 EU/kg were confirmed.

Intra-nodal and/or intra-lesion vaccine injection was performed as described by Koski, et al. Briefly, immunizations were administered in the National Institutes of Health-designated General Clinical Research Center at the Hospital of the University of Pennsylvania. Injections comprised 10-20 million HER2-pulsed DC1s suspended in 1 ml sterile saline, and administered by ultrasound guidance into groin lymph nodes, breast, or both. Immunizations were administered once weekly for 6 weeks, and all patients completed 6 immunizations. Immunization-related safety and toxicity data has been reported previously by Sharma, et al.

### Immune Response Detection

Circulating anti-HER2 CD4⁺ Th1 responses were generated from patient unexpanded peripheral blood mononuclear cells (“PBMCs”) pulsed with the six above-referenced HER2 class II binding peptides, by measuring IFN-γ production via enzyme-linked immunosorbent spot (“ELISPOT”) assays. ELISPOT was performed according to methods described by Koski, et al. Briefly, PVDF membrane plates (Mabtech Inc., Cincinnati, Ohio) were coated overnight with anti-IFN-γ capture antibody (1-D1K (Mabtech)). Cryopreserved PBMCs that were isolated using density gradient centrifugation, were thawed into pre-warmed DMEM medium supplemented with 5% human serum. After plates were washed and blocked, PBMCs were plated in triplicate (2×10⁵ cells/well), and the plates were incubated at 37° C. for 24-36 hours with either HER2-derived Class II binding peptides (4 μg) (peptide 42-56 (SEQ ID NO: 1); peptide 98-114 (SEQ ID NO:2); peptide 328-345 (SEQ ID NO:3); peptide 776-790 (SEQ ID NO:4); peptide 927-941 (SEQ ID NO:5); and peptide 1166-1180 (SEQ ID NO:6), media alone (unstimulated control), or positive control (anti-human CD3 and anti-CD28 antibodies (0.5 μg/mL each), both BD Pharmingen, San Diego, Calif.). After washing, detection antibody (7 B6-1-biotin (Mabtech); 100 μg/mL;) was added to each well, and the plates were incubated at 37° C. for 2 hours. Next, 1:1000 diluted streptavidin-horseradish peroxidase in PBS+0.5% FCS was added before incubation for an additional 1 hour at 37° C. TMB substrate solution (Kirkegaard & Perry Laboratories, Gaithersburg, Md.) was then added to reveal spot formation. After color development, wells were washed with tap water. Spot forming cells (“SFC”) were counted using an automated ELISPOT reader (ImmunoSpot CTL, Cleveland, Ohio).

Additionally, recall Th1 responses were examined by stimulating evaluable PBMCs from specific patient subsets with 1:100-diluted recall stimuli Candida albicans (Allermed Laboratories. San Diego, Calif.) and tetanus toxoid (Santa Cruz Biotechnology, Dallas, Tex.). In order to determine the relative functional activity of T_{reg} and/or Th2 phenotypes, IL-10 production was measured by ELISPOT, as described by Guerkov, R. E., et al., J. Immunol. Meth. 279:111 (2003), with 2.5 μg/ml of anti-CD3 antibody used as a positive control.

Since inter-replicate variability in ELISPOT assays was low (data not shown), an empiric method of determining antigen-specific response was carried out. A positive response to an individual HER2 Class II peptide was defined as: (1) threshold minimum of 20 SFC/2×10⁵ cells in experimental wells after subtracting unstimulated background; and (2) at least a two-fold increase of antigen-specific SFCs over the background. Three separate metrics of CD4⁺ Th1 responses were defined for each patient group: (a) overall anti-HER2 responsivity (i.e., proportion of patients responding to ≧1 peptide) (“responsivity”), (b) mean number of reactive peptides (“response repertoire”), and (c) cumulative response across 6 peptides (reported as SFC/10⁶ cells) (“cumulative response”).

### Inter-Assay Precision of ELISPOT

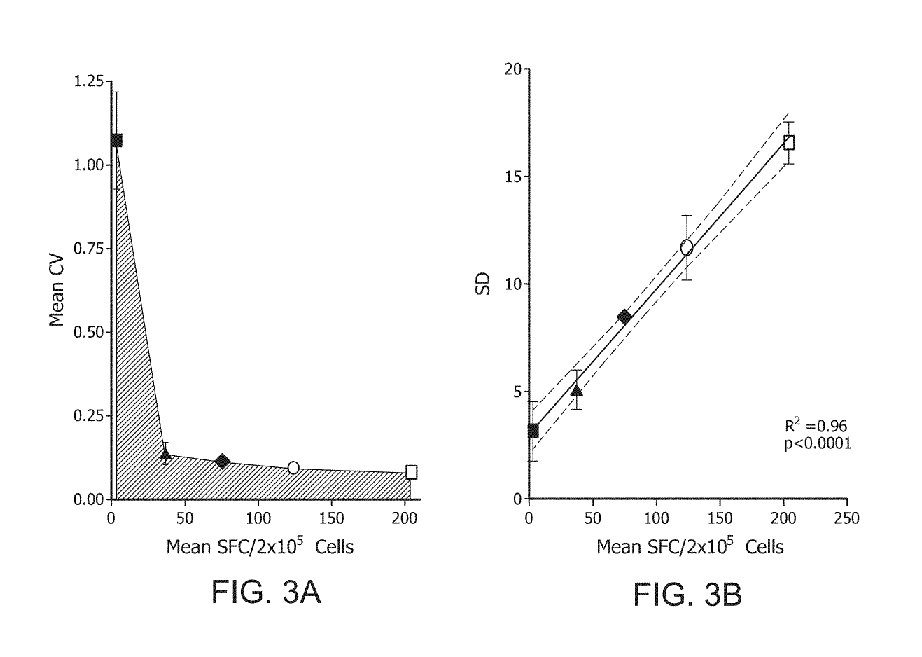

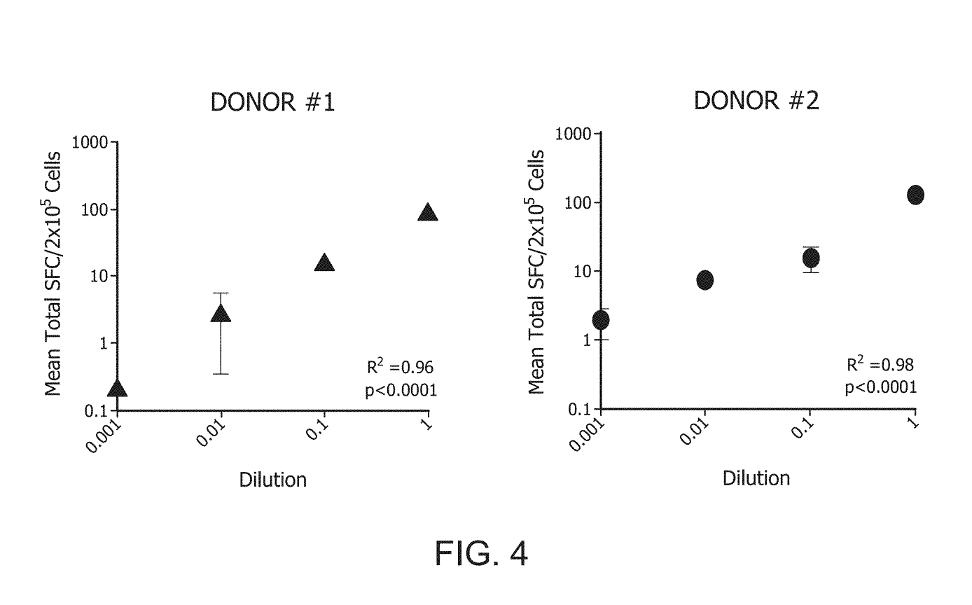

Inter-assay precision of ELISPOT was performed as described previously by Maecker, H. T., et al. BMC Immunology 9:9 (2008) (“Maecker, et al.”) When the mean coefficient of variance (“CV”) (three parallel replicates over three days) was plotted against cumulative Th1 response for five donors stimulated ex vivo with a HER2 extracellular domain (“ECD”) peptide mix (peptide 42-56 (SEQ ID NO: 1); peptide 98-114 (SEQ ID NO: 2); and peptide 328-345 (SEQ ID NO: 3)) a characteristic non-linear relationship was observed. Mean CV increased dramatically as cumulative response approached zero as shown in FIG. 3A. Due to the non-linear relationship between CV and cumulative response level, standard deviation (“SD”) of three assays on 3 separate days was plotted against cumulative Th1 response as a measure of inter-assay variability. Id. As shown in FIG. 3B, SD was found to be linearly related with the cumulative response (connecting line represents linear regression of the SD generated, with 95% confidence intervals of the regression shown with parallel dotted lines) (R²=0.96. p<0.0001).

Linearity studies were conducted in which triplicate samples of PBMCs donated from two high-responding HER2-reactive responders were serially diluted into PBMCs from a known allogenic non-HER2 responder, and stimulated ex vivo with a HER2 ECD peptide mix (peptide 42-56 (SEQ ID NO: 1); peptide 98-114 (SEQ ID NO: 2); and peptide 328-345 (SEQ ID NO: 3)). The same non-responding donor was used for all assays. Unstimulated background was subtracted for each dilution point. A significant linear relationship between Th1 response and dilution concentration was observed in both donors (#1: triangles; #2: circles). Collectively, these data suggest that EISPOT assays are precise, reliable, and reproducible.

### HER2 Antibody Detection

ELISA was performed to test patient sera for endogenous IgG1 and IgG4 anti-HER2 antibodies. EIA/RIA plates were coated with HER2 ECD peptides (5 μg/ml; Speed Biosystems, Rockville, Md.) in bicarbonate buffer, and incubated overnight at room temperature (“RT”). The following day, plates were blocked with 1% casein in PBS, sera (1:100 dilution) added in quadruplicate in blocking buffer, incubated for 2 hours, and washed three times before the addition of 1:500-diluted HRP-conjugated anti-human secondary antibody specific for either IgG1 or IgG4 (Life Technologies, Grand Island, N.Y.). After incubation for 1 hour, plates were washed and developed with TMB substrate solution (Kirkegaard & Perry Laboratories).

### Flow Cytometry

PBMC suspensions were prepared in FACS buffer (PBS+1% FCS+0.01% azide), and anti-human-CD3, -CD4, -CD8, -CD83, -HLA-DR, -CD11b, -CD33, -CD19, -CD56, -CD16 (all BD Biosciences, San Jose, Calif.), -CD4, and -CD25 (both Biolegend, San Diego, Calif.) were used to determine relative PBMC immunophenotype. After washing, cells were incubated for 30 minutes at RT with antibody mixtures. Following incubation, cells were washed three times with FACS buffer and fixed with 2% paraformaldehyde. Stained samples were analyzed within 24 hours. Intracellular staining of PBMCs with anti-FoxP3 (eBioscience, San Diego, Calif.) using a FoxP3 fixation/permeabilization kit (Biolegend) was performed according to the manufacturer's instructions. Flow cytometric analysis was performed using a BD LSR-II cytometer, and datasets were analyzed using CellQuest Pro™ software (BD Biosciences).

### Pathologic Staining

Formalin-fixed, paraffin-embedded tissue blocks from HER2^{pos}-DCIS and -IBC tumors were sectioned and stained with hematoxylin and eosin (“H&E”) to assess peritumoral lymphocytic infiltrates. Multiplex-labeled IF (PerkinElmer, Waltham, Mass.) was used to examine lymphocyte subpopulations in sample cases from HER2^{pos}-DCIS and -IBC tumors (see, Wang. C., et al., Journal for Immunotherapy of Cancer 118:1(Suppl. 1) 54 (2013) (“Wang, C., et al.”). Tumors were stained for CD4, CD8, CD20, and 4′,6′-diamino-2-phenylindole (“DAPI”) with same-species fluorescence labeling using tyramide signal amplification. Images were analyzed using a Vectra multispectral microscope with pattern recognition software to identify tumor, stroma, and T-/B-lymphocytes.

### Apoptosis Assays

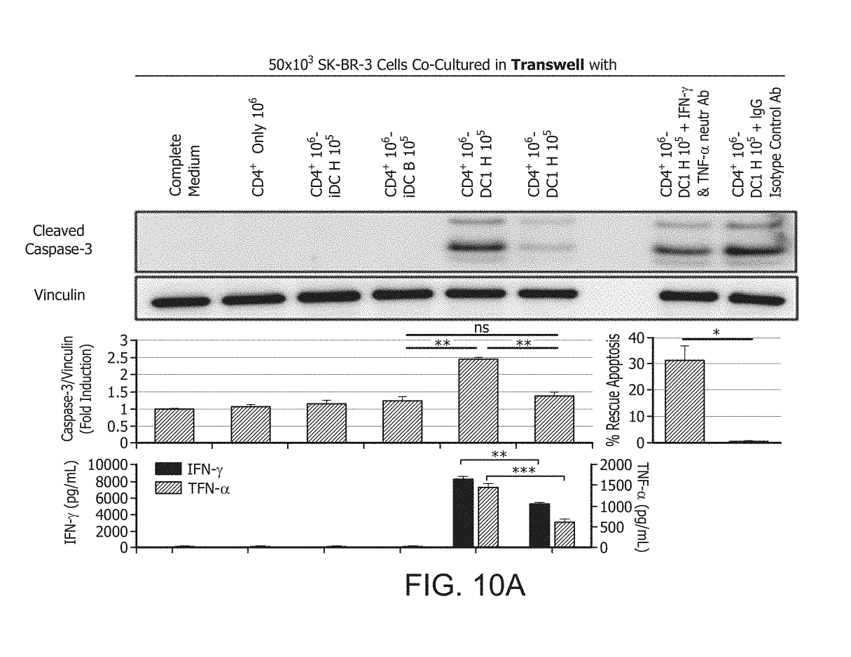

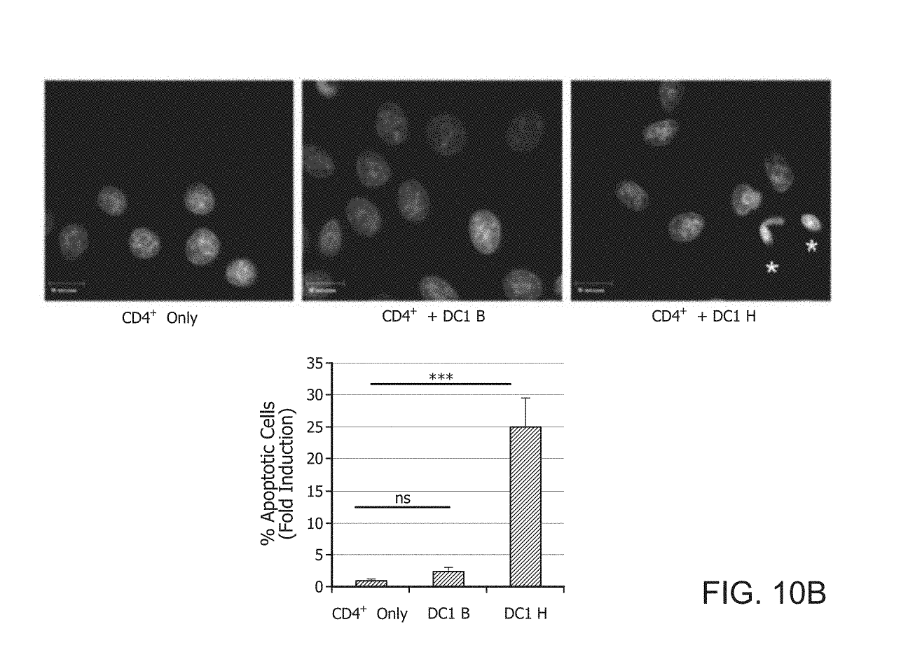

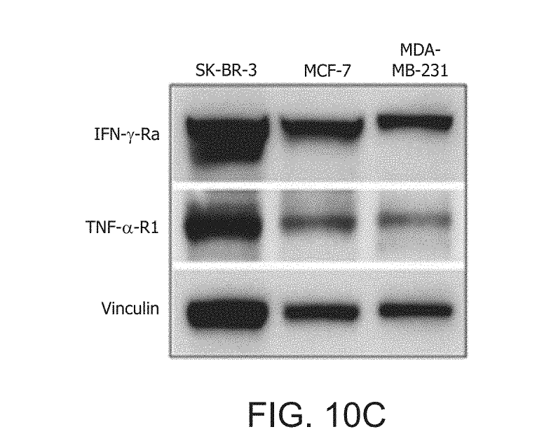

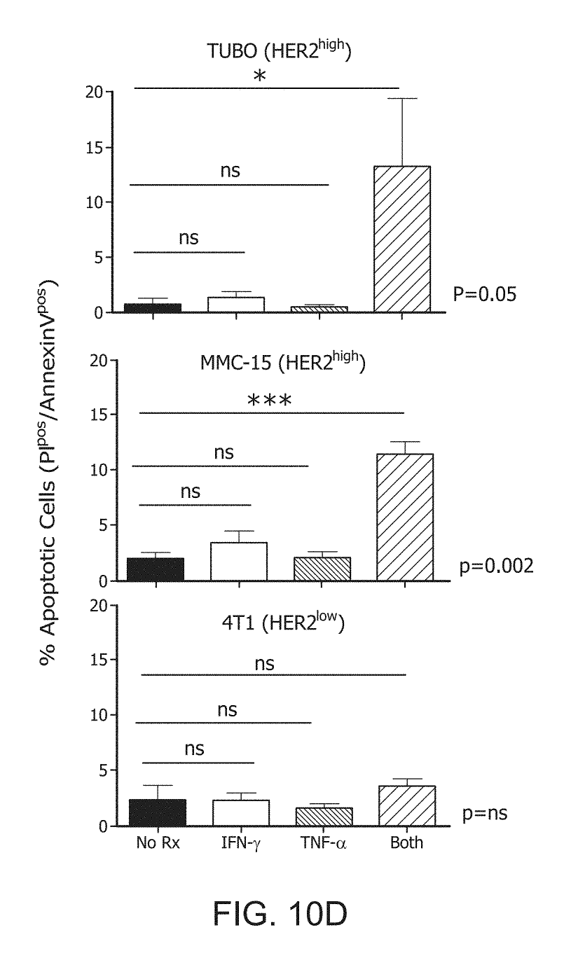

BC cell lines with a spectrum of HER2 expression (Ithimakin, S., et al., Can. Res. 73:1635-46 (2013))—HER2^{high} SK-BR-3, HER2^{intermediate} MCF-7, HER2^{low} MDA-MB-231 (American Type Culture Collection)—were cultured in RPMI-1640+10% FBS (Cellgro/Mediatech, Manassas, Va.). 50×10³ BC cells were plated in a transwell system (BD Biosciences), and co-cultured with 10⁶ CD4⁺ T-cells and 10⁵ DC1s (mature DCs) or iDCs (immature DCs). DC1s, iDCs and CD4⁺ T-cells were obtained from select post-vaccinated patients as described by Sharma, et al. DC1s/iDCs were pulsed with Class II HER2 or irrelevant control BRAF peptides (20 μg/ml) for 24 hours at 37° C. Specifically, as shown in FIG. 10A, 50×10³ SK-BR-3 cells were co-cultured with medium alone (“complete medium”), 10⁶ human CD4⁺ T-cells alone (“CD4⁺ only”), 10⁶ CD4⁺ T-cells+10⁵ each of HER2 Class II peptide (“iDC H”)- or irrelevant Class II BRAF peptide (“iDC B”)-pulsed iDCs, and 10⁶ CD4⁺ T-cells+10⁵ each HER2 (“DC1 H”)- or BRAF (“DC1 B”)-pulsed DC1s. DC1s/iDCs were pulsed with Class II HER2 or irrelevant control BRAF peptides (20 μg/ml) for 24 hours at 37° C. Control wells contained culture medium or CD4⁺ T-cells only.

Polyclonal goat IgG anti-human TNF-α (0.06 μg/mL per 0.75 ng/mL TNF-α) and IFN-γ (0.3 μg/mL per 5 ng/mL IFN-γ) antibodies (R&D Systems, Minneapolis, Minn.) were used to neutralize Th1 cytokines, with goat IgG isotype as control. Following treatments, BC cells were lysed and subjected to western blot analysis for cleaved caspase-3 detection. Degree of nuclear fragmentation was assessed by DAPI staining. Additionally, apoptosis in 50×10 BC cells incubated with (i) supernatants from iDC:CD4⁺ or DC1:CD4⁺ T-cell co-cultures, or (ii) TNF-α (10-200 ng/mL as indicated)+IFN-γ (100-2000 U/mL as indicated) (R&D Systems) was examined by cleaved caspase-3 detection.

Transgenic murine mammary carcinoma lines expressing high levels of rodent HER2/ErbB2 (HER2^{high} TUBO and MMC15 [the latter a generous gift of Li-Xin Wang, Cleveland Clinic]) and HER2^{low/neg} (4T1) were incubated with medium (RPMI-1640+10% FCS) alone, recombinant mouse rmTNF-α (1 ng/ml; Peprotech) alone, rmIFN-γ (12.5 ng/ml; Peprotech) alone, or combination rmTNF-α+rmIFN-γ for 72 hours at 37° C. Following trypsinization, harvested cells were washed and resuspended in FACS buffer, and FITC-Annexin V (4 μl) and P1 (2 μl) added. Cells were incubated at 4° C. for 20 minutes, washed twice, and subjected to flow cytometry. Apoptotic cells were defined as those staining positive for both markers. Vinculin was used as a loading control. Corresponding mean caspase-3/vinculin ratios±SEM, indicating fold induction of apoptosis, were quantified using ImageJ software.

### ELISA

Capture and biotinylated detection antibodies and standards for IFN-γ and TNF-α (BD Pharmingen) were used according to the manufacturer's protocols.

### Statistical Analysis

Descriptive statistics were employed to summarize distributions of patient characteristics and immune response variables. Continuous variables were summarized by mean, SEM, and range and categorical variables by frequency and percentage. Data transformation (natural log or square root) was applied, when necessary, to meet assumptions of parametric testing. ANOVA with post-hoc Scheffé paired testing (parametric) or Kruskal-Wallis testing (non-parametric) were employed to compare continuous variables for >3 groups. Student's t-test was used for 2-group comparisons. Fisher's exact test was employed to compare categorical variables in multi-level tables. Student's paired t-test and McNemar's exact test were used to evaluate within-patient paired changes (e.g., pre-vaccination vs. post-vaccination) in Th1 response variables. A p-value p≦00.05 was considered statistically significant. All tests were two-sided. Statistical analyses were performed in either SPSS (IBM Corp.). or StatXact (Cytel Corp. San Diego, Calif.).

### Results

### Patient Characteristics

After random consecutive enrollment, 143 subjects met study inclusion criteria. Mean age of participants was 53.1±1.4 (range, 21-88) years and a majority (79.0%) were Caucasian. Patient/donor cohorts, with time-points at which blood was drawn, are indicated in FIG. 1 and Methods section above. Donors' demographic and tumor-related characteristics of study participants are detailed in Table 1 above. Twenty-six (83.9%) and 11 (50.0%) patients in the HER2^{pos}-DCIS and -IBC cohorts, respectively, were previously enrolled in neoadjuvant type 1-polarized (“DC1”) vaccination trials for HER2^{pos}-DCIS; their patient/tumor characteristics have been reported by Sharma, et al.

Loss of Systemic Anti-HER2 Th1 Immunity Correlates with Progression of Breast Tumorigenesis

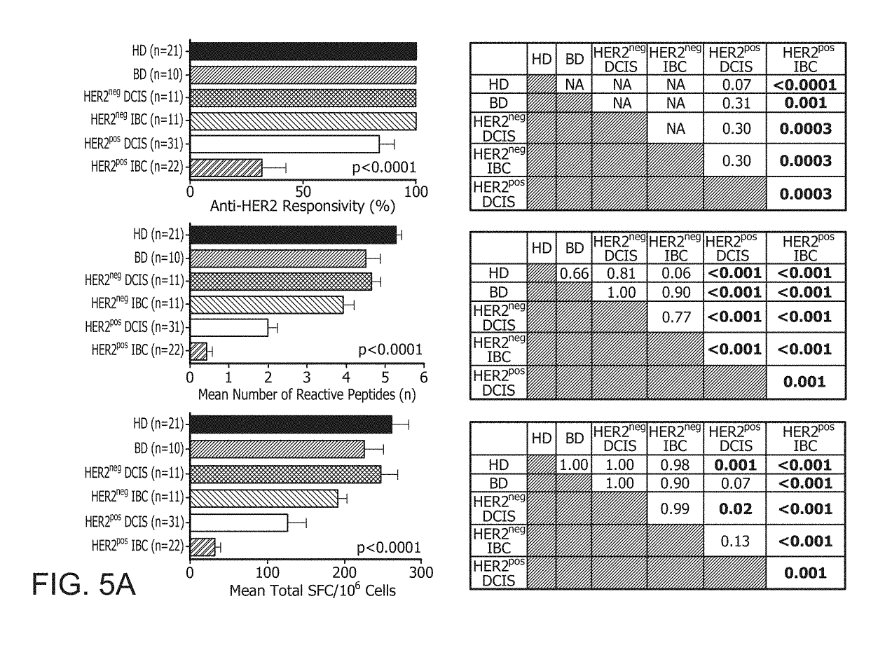

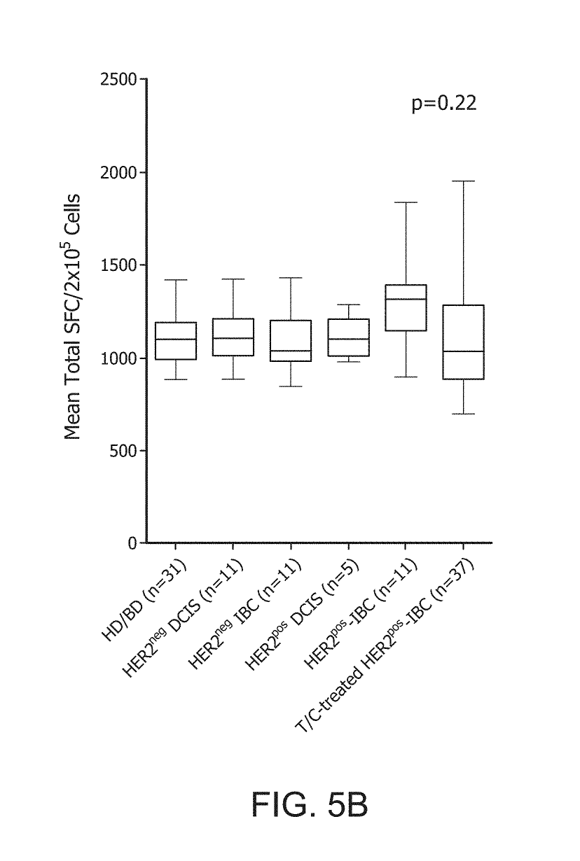

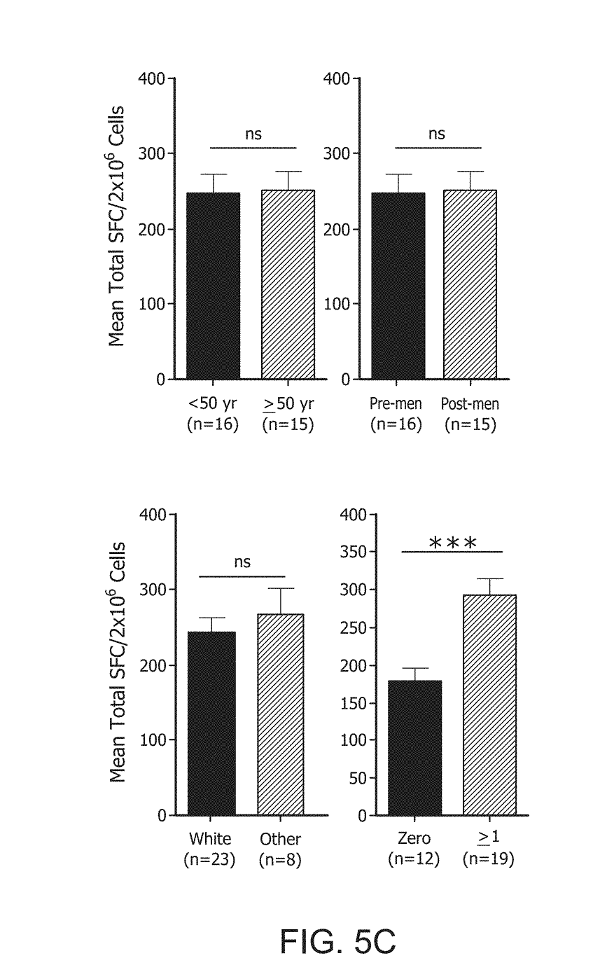

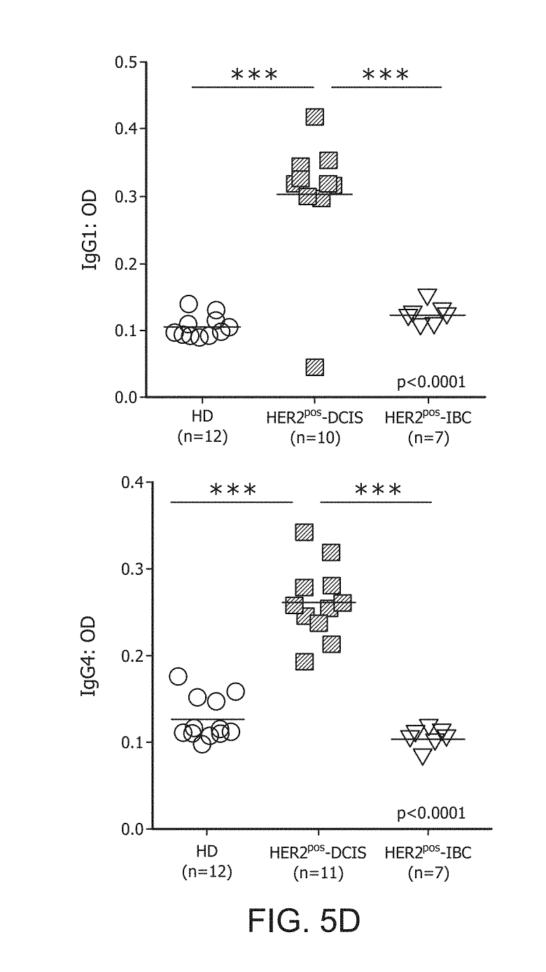

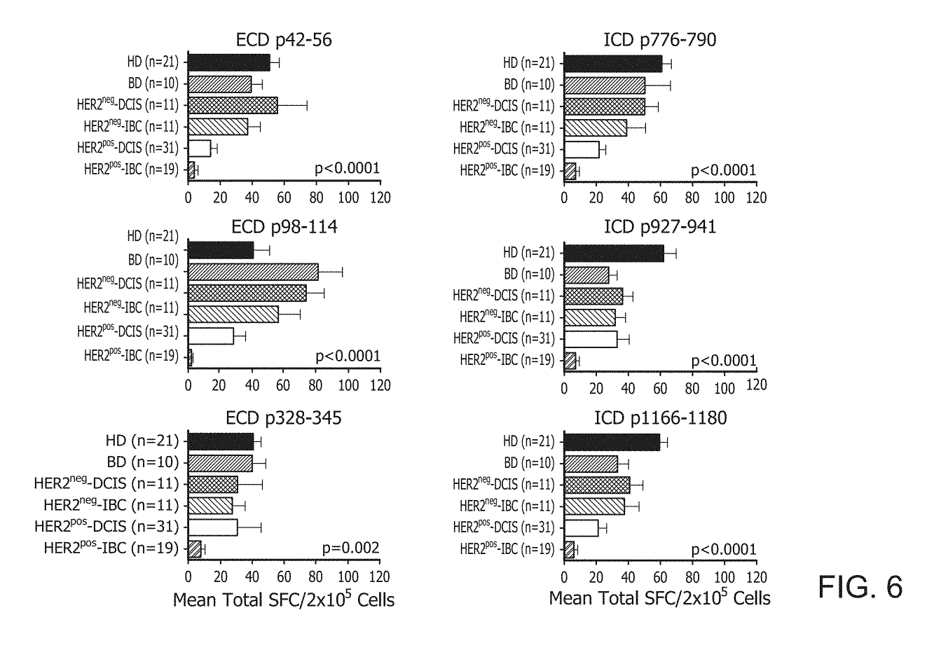

Using peripheral blood mononuclear cells (“PBMCs”), variations in systemic anti-HER2 CD4⁺ Th1 response across a tumorigenesis continuum were examined prospectively by ex vivo HER2 peptide-stimulated IFN-γ ELISPOT assays. Three Th1 response metrics were compared between groups: (a) overall anti-HER2 responsivity (proportion of patients responding to ≧1 peptide), (b) mean number of reactive peptides (repertoire), and (c) cumulative response across 6 class II peptides described above. When compared with healthy donors (“HD”) or patients with benign breast disease (“BD”) (FIG. 1, cohort A), a significant stepwise decline in Th1 response was observed in HER2^{pos} breast cancer patients. Beginning in treatment-naïve HER2^{pos}-DCIS (FIG. 1, cohort C) and reaching a low point in treatment-naïve Stage I/II HER2^{pos}-IBC (FIG. 1, cohort F), this progressive loss of Th1 immunity was observed uniformly across all Th1 response metrics. For instance, the overall anti-HER2 responsivity decreased from 100% in HD/BD to 84% in HER2^{pos}-DCIS to 32% in HER2^{pos}-IBC patients (p<0.0001). Similar significant stepwise decrements in response repertoire (5.2±0.2 vs. 4.5.0.4 vs. 2.0±0.3 vs. 0.4±02; p<0.0001), and cumulative response (259.9±23.5 vs. 225.1±25.5 vs. 126.1±24.4 vs. 32.3±5.4 spot-forming cells (“SFC”)/10⁶ cells, p<0.0001) were observed across HD, BD, HER2′⁺-DCIS, and Stage I/II HER2^{pos}-IBC patients, respectively, as shown in FIG. 5A. On post-hoc comparison, Th1 responses in HER2^{pos}-DCIS patients were significantly lower than in HDs when assessed by response repertoire (p<0.001) and cumulative response (p=0.001) but not overall responsivity (p=0.07). Th1 responses in HER2^{pos}-IBC patients were further suppressed in that these patients had significantly lower overall responsivity (p=0.0003), repertoire (p=0.001), and cumulative response (p<0.001) compared with HER2^{pos}-DCIS patients. The percentage of reactive cells per 10⁶ PBMCs ranged from 0.03% in HD to 0.003% in HER2^{pos}-IBC patients.

It is to be noted that Th1 responses in treatment-naïve HER2^{neg}-DCIS (FIG. 1 cohort B) or HER2^{neg}-IBC (FIG. 1 cohort D) patients and HD/BD patients did not vary appreciably. Compared with HER2^{neg}-DCIS patients, however, HER2^{pos}-DCIS patients demonstrated significantly lower anti-HER2 Th1 repertoire (p<0.001) and cumulative response (p=0.02). Similarly, compared with HER2^{neg}-IBC patients, HER2^{pos}-IBC patients had lower responsivity (p=0.0003), repertoire (p<0.001), and cumulative response (p<0.001) as seen in FIG. 5A.

Individual HER2 peptide-specific contributions to cumulative Th1 responses across patient groups demonstrated similar stepwise Th1 decrements from HD/BD to HER2^{pos}-IBC patients, across all HER2 extracellular domain (“ECD”) and intracellular domain (“ICD”) peptide reactivity profiles (p<0.0050) as shown in FIG. 6. Disproportionate focusing of Th1 immune responses towards a selective HER2 epitope(s) in DCIS/IBC patients may not explain the progressive Th1 loss in HER2^{pos} tumorigenesis.

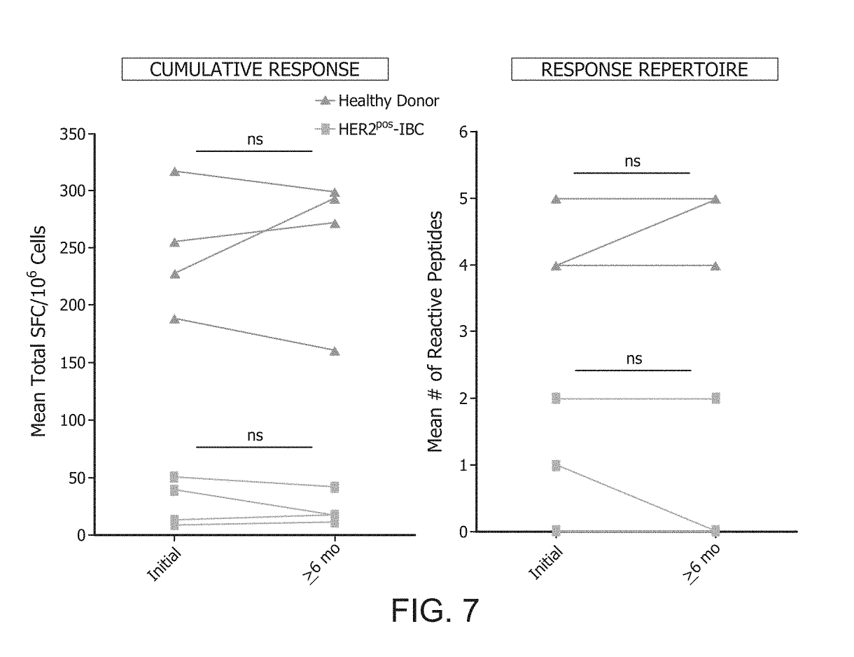

In order to investigate if Th1 responses in HD/BD donors were disproportionately higher in certain subgroups, responses were compared by age (<50 yr (n=16), ≧50 yr (n=15)), menopausal status (pre-menopausal (n=16), post-menopausal (n=15)), race (White (n=23), other (Black/Asian/etc.; n=8)), or gravidity (zero (n=12), ≧1 (n=19) pregnancies). No significant differences in anti-HER2 Th1 repertoire or cumulative response were observed in HD subgroups stratified by age, race, or menopausal status; however, gravid donors (i.e. ≧1 pregnancies) had a significantly higher anti-HER2 Th1 repertoire (5.3±0.2 vs. 4.6±0.2, p=0.01) and cumulative response (293.1±21.2 vs. 178.219.0, p=0.0008) compared with non-gravid donors (FIG. 5C). Temporal variability in Th1 responses was examined in HD/BDs and HER2^{pos}-IBC donors (n=4 each); in blood drawn from the same patients at ≧6 month intervals, relatively unchanged Th1 repertoires and cumulative responses were observed over time as seen in FIG. 7.

### Anti-HER2 IgG1 and IgG4 Antibody Responses are Lost in HER2^{pos}-IBC

After noting pre-existing anti-HER2 Th1 responses in HDs that decay in HER2^{pos} breast tumorigenesis, serum reactivity was examined against recombinant HER2 ECD peptides using available sera from HDs, HER2^{pos}-DCIS and HER2^{pos}-IBC patients. Both IgG1, associated with Th1 immunity, and IgG4, associated with chronic antigen exposure, were evaluated. Compared with HDs (n=12) and treatment-naïve HER2^{pos}-IBC patients (n=7), a relative increase in both anti-HER2 IgG1 and IgG4 (both p<0.0001) levels was observed in HER2^{pos}-DCIS patients (n=10 IgG1, n=11 IgG4) by ELISA as shown in FIG. 5D. Comparatively lower anti-HER2 antibody levels in HER2^{pos}-IBC patients suggest that endogenous anti-HER2 response is lost upon disease progression.

CD4⁺ Th1 Response in Equivocal HER2-Expressing IBC Differs Significantly from Non-Equivocal HER2^{neg}-IBC

Th1 profiles in HER2^{neg}-IBC patients were examined in order to identify subgroups with a relative decline in anti-HER2 Th1 immunity. When compared with non-equivocal HER2^{neg}-IBC (IHC 0/1+) patients (n=11), equivocal HER2-expressing (IHC 2+/FISH negative) IBC patients (n=7) demonstrated significantly lower overall responsivity (28.6% [IHC 2+] vs. 100% in [IHC 0/1+], p=0.002), repertoire (0.3±0.2 vs. 3.9±0.3, p<0.0001), and cumulative response (21.4±6.5 vs. 191.2±11.7 SFC/10⁶ cells, p=0.002). Th1 responses in equivocal HER2-expressing IBC patients resembled those seen in HER2^{pos}-IBC patients represented in FIG. 5A. IL-10 production measured via ELISPOT and the relative proportion of T_{reg} (CD4⁺CD25⁺FoxP3⁺) cells by flow cytometry did not differ significantly between equivocal and non-equivocal HER2^{neg}-IBC patients (data not shown).

### Th1 Response Loss is not Related to Host-Level T-Cell Anergy or Increasingly Immunosuppressive Circulating Immune Phenotype

Immunocompetence in evaluable donor subgroups was assessed by measuring Th1 response to anti-CD3/anti-CD28 via IFN-γ ELISPOT; these responses also served as donor-specific positive controls in all ELISPOT assays. Median anti-CD3/CD28 responses did not differ (1098 vs. 1104 vs. 1032 vs. 1099 vs. 1318 vs. 1032 SFC/2×10⁵ cells, p=0.22) between HD/BD (n=31), HER2^{neg}-DCIS (n=11), HER2^{neg}-IBC (n=11), HER2^{pos}-DCIS (n=S) HER2^{pos}-IBC (n=11), and T/C-treated HER2^{pos}-IBC (n=37) cohorts, respectively, as seen in FIG. 5B. Moreover, Th1 responses to recall stimuli [tetanus toxoid (105±17.0 vs. 96±15.6 vs. 101±11.3 SFC/2×10⁵), and Candida albicans (185±10.2 vs. 199±15.3 vs. 181±14.6 SFC/2×10⁵)] were similar between evaluated HD (n=10), HER2^{pos}-IBC (n=11), and T/C-treated IBC (n=10) cohorts, respectively, as seen in FIG. 8A. Collectively, these data suggest that the progressive anti-HER2 Th1 response loss in HER2-driven BC is not attributable to host-level T-cell anergy or impaired antigen-presenting capacity in IBC patients' PBMCs.

Using flow cytometry, the mean proportion of CD3+CD4+(72.8±2.3% vs. 62.6±3.2% vs. 63.3±6.9%, p=0.26) and CD3+CD8+(25.1±2.9% vs. 37.9±4.7% vs. 38.2±6.6%, p=0.15) cells did not differ significantly between PBMCs from HDs, HER2pos-IBC, and T/C-treated HER2pos-IBC cohorts, respectively as is shown in FIG. 8B. No differences in proportions of B-cells (CD19+) or natural killer (NK) cells (CD3-CD16+) were observed between groups (data not shown). Systemic immunosuppressive phenotypes were then compared between the following groups. As shown in FIG. 8C mean proportions of CD4+CD25+FoxP3+ cells (Treg) (1.8±0.3% vs. 1.5±0.2% vs. 1.7±0.3%, p=0.78), and CD11b+CD33+HLA-DR−CD83− cells (Myeloid-derived Suppressor Cells “MDSCs”) (0.6±0.1% vs. 1.0±0.3% vs. 0.9±0.1%, p=0.33) did not differ significantly between HD, HER2pos-IBC, and T/C-treated HER2pos-IBC subgroups, respectively.

HER2-specific IL-10 production, a surrogate for T-helper type 2 (“Th2”) and/or T_{reg} function, was also examined across patient subgroups via ELISPOT. FIG. 8D shows anti-HER2 responsivity (all 100%), repertoire (1.8±0.4 vs. 1.8±0.2 vs. 2.0±0.3), and cumulative response (77.4±15.2 vs. 66.6±8.2 vs. 92.8±4.7) did not differ significantly between HD, HER2^{pos}-IBC, and T/C-treated IBC cohorts, respectively. FIG. 8E shows IL-10 production to anti-CD3 stimulus was similar across all evaluated groups. While overall IL-10 production did not differ between subgroups, donor-matched HER2-specific IFN-γ:IL-10 production ratios dramatically shifted from 6.6:1 (relative Th1-favoring phenotype) in HDs to 0.74:1 and 0.97:1 (relative T_{reg}/Th2-favoring phenotype) in untreated and T/C-treated HER2^{pos}-IBC patients, respectively (p=0.009) (top panel).

### Systemic Th1 Response Loss is Unrelated to Disproportionate T-Lymphocyte Trafficking to HER2^{pos}-IBC Lesions

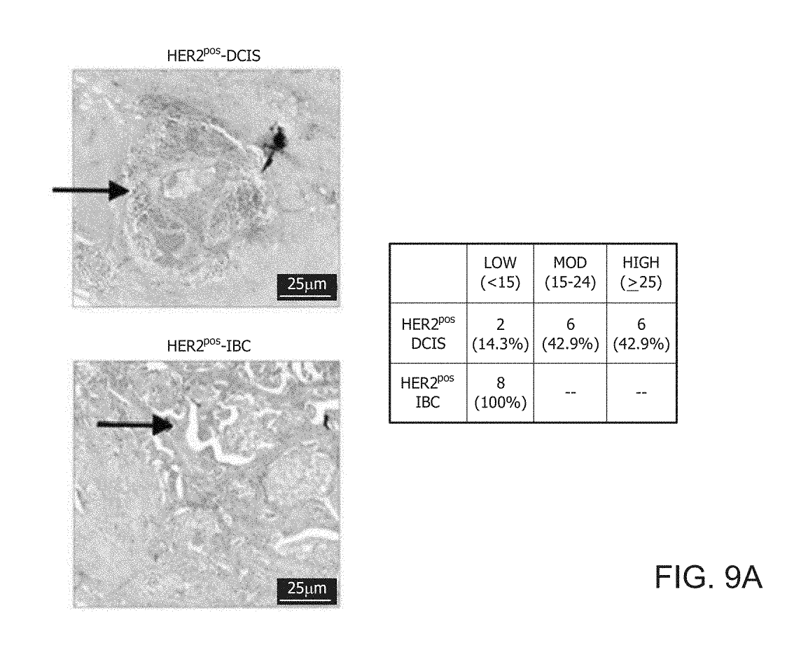

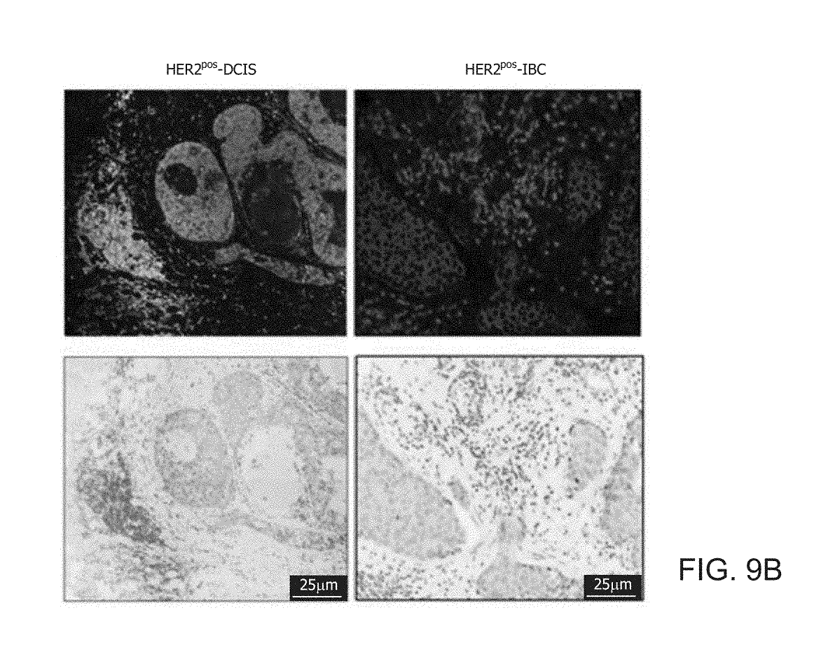

Immunohistochemical (“IHC”) analysis of 14 HER2^{pos}-DCIS and 8 HER2^{pos}-IBC lesions, available for pathologic review, was performed to determine if the systemic IFN-γ^{pos} CD4⁺ response loss was related to disproportionate lymphocyte trafficking to IBC lesions. The results are shown in FIG. 9A. Whereas moderate (≧15% stromal involvement) to high (≧25%) lymphocyte levels were observed aggregating in stromal regions outside DCIS-containing ducts in a majority (12/14; 85.7%) of evaluable patients (top) (shown by arrow), a relative paucity of lymphocytes (arrow) was seen around invasive foci in all 8 IBC patients 98/8; 100%) (bottom).

Lymphocytic phenotypes were analyzed by a novel multiplex-labeled immunofluorescence (“IF”) imaging technique which discriminates tumor and stromal regions, and reliably detects relative CD4⁺ (green signal), CD8⁺ (yellow), and CD20⁺ (red) subpopulations as described by Wang, C., et al. The results are shown in FIG. 9B. A majority of stromal (“StL”) and tumor-infiltrating lymphocytes (“TIL”) in HER2^{pos}-IBC tumors comprised CD8⁺ cells (upper right panel). Moreover, a relative paucity of CD4⁺ TIL/StL was observed in HER2^{pos}-IBC tumors compared with DCIS lesions (upper left panel). Disproportionate peritumoral CD4⁺ T-cell trafficking to HER2^{pos}-IBC lesions may not explain the systemic depletion of IFN-γ^{pos} CD4⁺ T-cell subsets.

High/Intermediate HER2-Expressing, but not Low HER2-Expressing. BC Cells are Susceptible to CD4⁺ Th1-Mediated Apoptosis

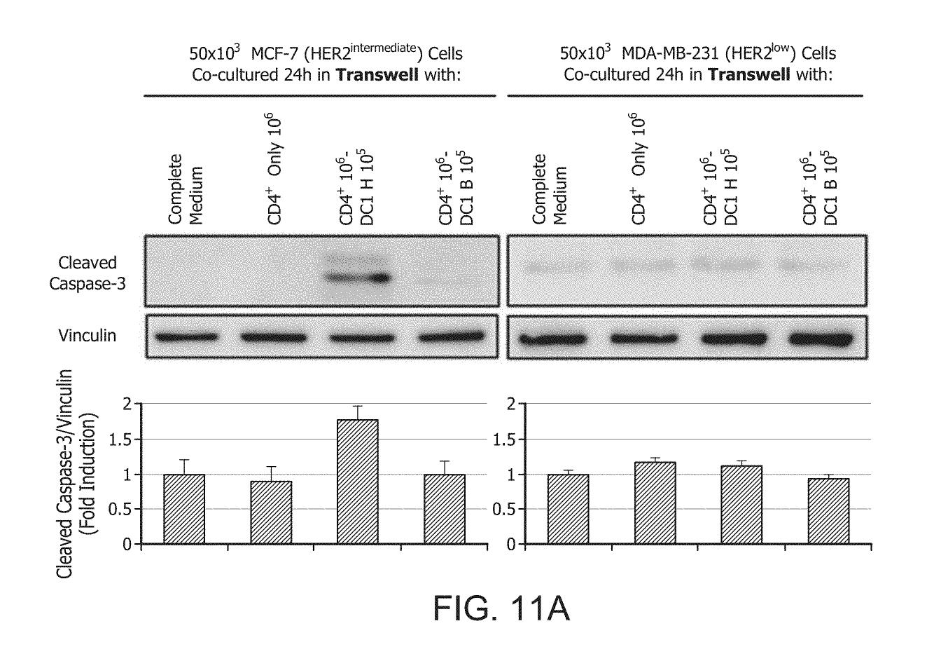

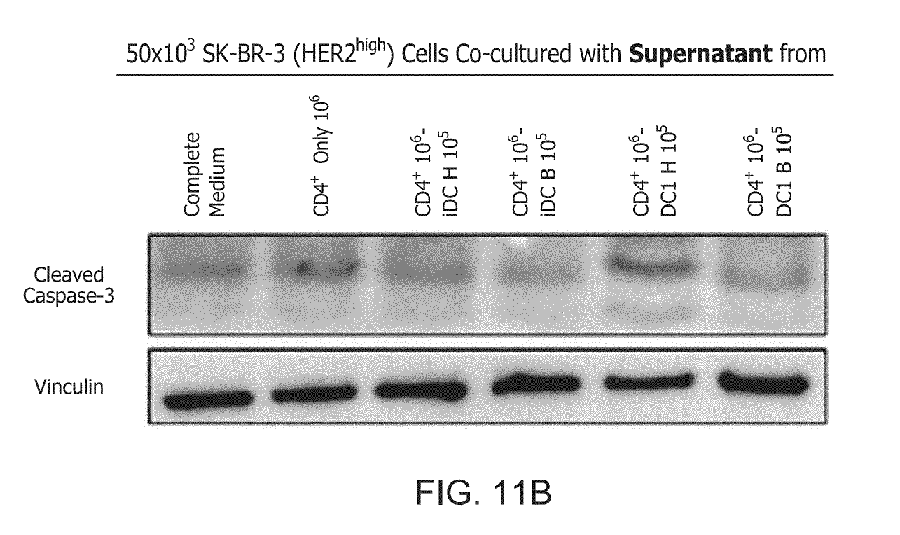

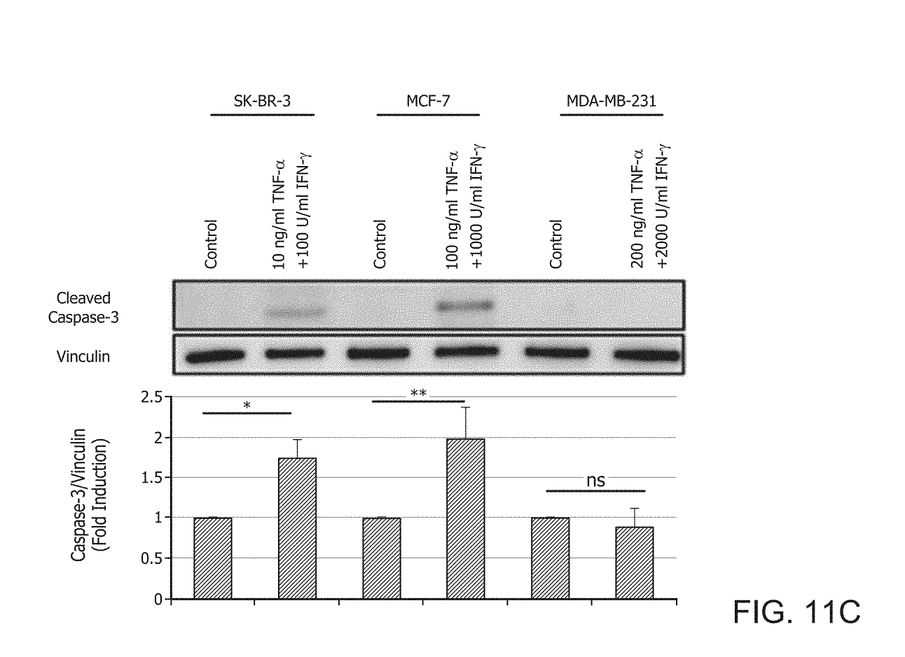

Th1-mediated effects on HER2^{high} SK-BR-3, HER2^{intermediate} MCF-7, and HER2^{low} MDA-MB-231 BC cell lines in vitro were also evaluated. Co-culture of increasing proportions of HER2 Class II peptide-specific CD4⁺ Th1 cells, sensitized with HER2-pulsed DC1, with the above types of HER-expressing BC cells using a transwell culture system resulted in striking dose-dependent apoptosis of SK-BR-3 evidenced by increased caspace-3 detection by western blot analysis shown in FIG. 10A and MCF-7, but not MDA-MB-231, BC cells as seen in FIG. 11A. In contrast, apoptosis was relatively insignificant in BC cells co-cultured with CD4⁺ T-cells sensitized by immature DCs (iDC H and iDC B) or control Class II peptide (BRAF)-pulsed DC1s (DC1 B's) as seen in FIGS. 10A and 11A. Quantification of Th1 cytokines elaborated in these co-culture supernatants by ELISA indicated significantly increased IFN-γ and TNF-α production from CD4⁺ T-cell:HER2-pulsed DC1, compared with CD4⁺:BRAF control-DC1, co-cultures as shown in FIG. 10A, corresponding with the degree of apoptosis observed.

A similarly specific apoptosis was observed in SK-BR-3 cells when incubated with supernatants from CD4⁺ T-cell:HER2-pulsed DC1 co-cultures, but not CD4⁺:HER⁺2-iDC or CD4⁺:BRAF control-DC1 co-cultures as shown in FIG. 11B. Compared with controls, HER2-specific Th1 cells resulted in a 25-fold increase in SK-BR-3 apoptosis as evidenced by DAPI staining as seen in FIG. 10B, right photograph and bar graph. Taken together, these data suggest that anti-HER2 CD4⁺ Th1 cells produce soluble factors that mediate apoptosis of high/intermediate HER2-expressing, but not low HER2-expressing, breast cancer cells.

Importantly, HER2^{high} SK-BR-3 apoptosis could be significantly rescued by neutralizing IFN-γ and TNF-α, as seen in FIG. 10A, suggesting a critical role for pleiotropic Th1 cytokines in mediating HER2-specific cellular apoptosis. To explore these observations further, the impact of IFN-γ and TNF-α treatment on BC cells were examined. Regardless of HER2 expression, human BC cells uniformly maintained IFN-γ and TNF-α receptor expression as seen in FIG. 10C. IFN-γ and TNF-α treatment resulted in significant apoptosis of HER2^{high} SK-BR-3 and HER2^{intermediate} MCF-7, but not HER2^{low} MDA-MB-231, cells as seen in FIG. 1 IC. Next, to assess if reinstatement of HER2 expression in MDA-MB-231 cells restored susceptibility to Th1 cytokine-mediated apoptosis, MDA-MB-231 cells were stably transfected with a wild-type HER2 plasmid (pcDNA-HER2) or with control empty vector (pcDNA3; kind gifts of Mark I. Greene, University of Pennsylvania) and treated with IFN-γ and TNF-α (2000 U/ml and 200 ng/ml, respectively; doses equivalent to those used against MDA-MB-231 cells in FIG. 1 IC). Significant IFN-γ/TNF-α-induced apoptosis was observed in HER2-transfected, but not vector-transfected, MDA-MB-231 cells (data not shown).

Finally, this Th1 cytokine-mediated HER2-specific apoptosis was corroborated in transgenic murine mammary carcinoma cells. Dual treatment with recombinant mouse IFN-γ and TNF-α, but not with either cytokine alone, resulted in significant apoptosis of HER2^{high} TUBO and MMC15, but not HER2^{low/neg} 4T1, cells as seen in FIG. 10D.

Th1 Response Loss in HER2^{pos}-IBC is Restored after HER2-Pulsed DC Vaccination, but not Following HER2-Targeting or Conventional Therapies

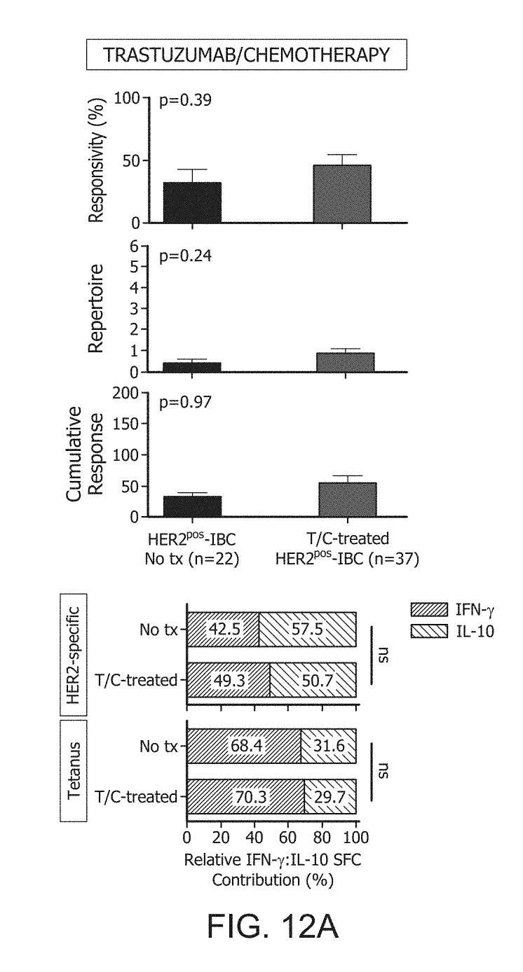

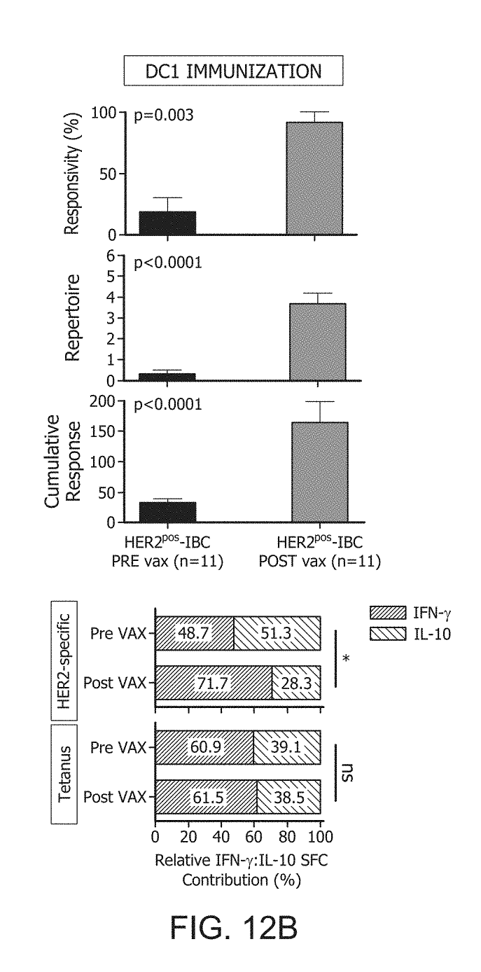

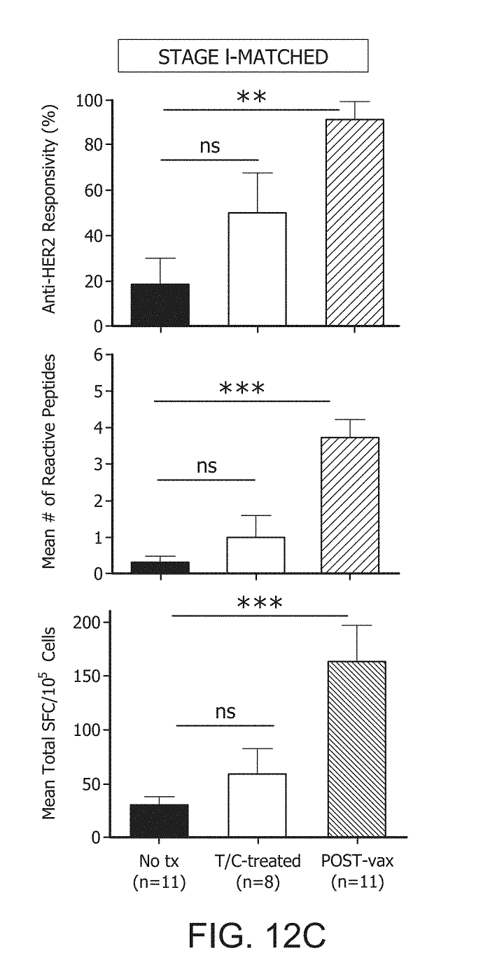

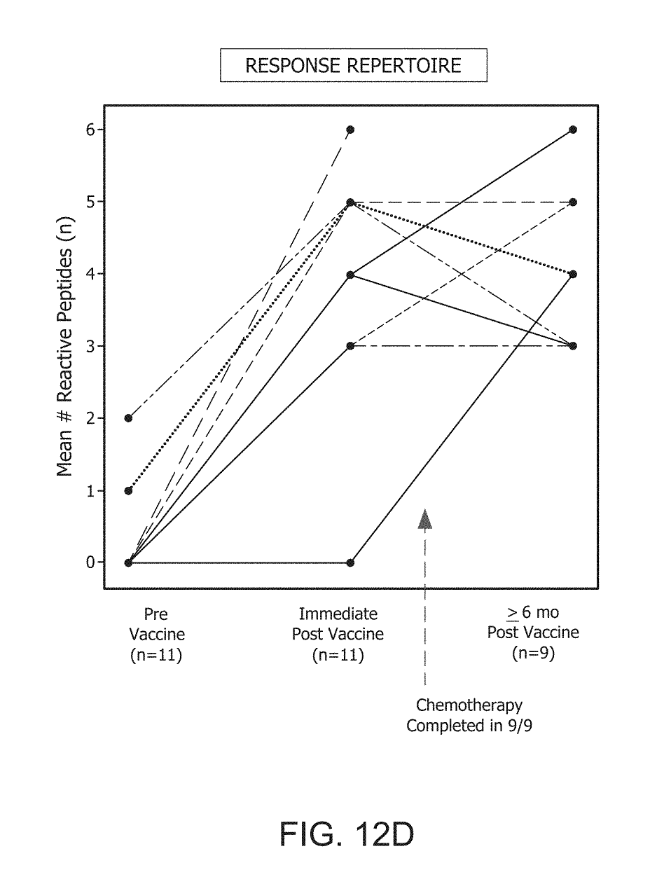

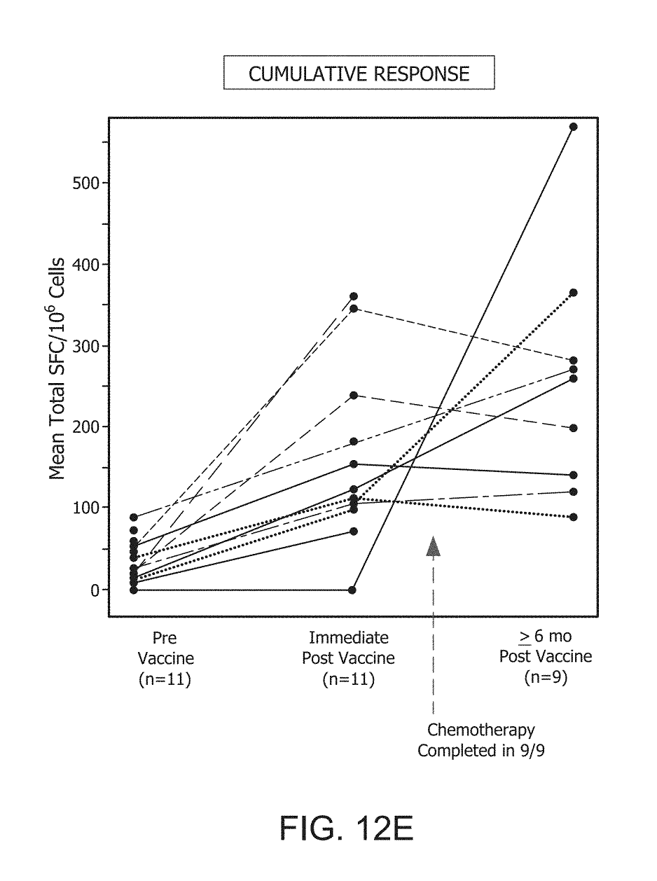

Differential effects following T/C treatment and HER2-pulsed DC1 immunization on Th1 responses in HER2^{pos}-IBC patients were analyzed and the results are shown in FIG. 12A, top panels. Treatment-naïve stage I/II HER2^{pos}-IBC patients (n=22; FIG. 1 cohort F) and T/C-treated stage I-III HER2^{pos}-IBC patients (n=37; FIG. 1 cohort G) did not differ significantly in anti-HER2 responsivity (31.6% untreated vs. 45.9% T/C-treated, p=0.39), repertoire (0.4±0.2 vs. 0.8±0.2, p=00.24), or cumulative response (32.3±5.4 vs. 54.5±12.0 SFC/10⁶, p=0.97). As shown in FIG. 12B, top panels, following HER2-pulsed DC1 vaccination in 11 Stage I HER2^{pos}-IBC patients (FIG. 1 cohort H), however, significant improvements were observed in anti-HER2 responsivity (18.2% pre-vaccine vs. 90.9% post-vaccine, p=0.0035), repertoire (0.3±0.2 vs. 3.7±0.5, p<0.0001), and cumulative response (29.7±7.9 vs. 162.8±33.7 SFC/10⁶, p<0.0001). The striking Th1 restoration effect following DC1 vaccination, but not after T/C receipt, persisted on stage-matched comparison between Stage I treatment-naïve (n=11), T/C-treated (n=8), and vaccinated (n=11) HER2^{pos}-IBC patients as seen in FIG. 12C.

Differences in relative proportions of IFN-γ^{pos}:IL-10^{pos} reactive T-cells were examined following DC1 vaccination compared with T/C treatment. In concurrently performed donor-matched comparisons, while both HER2-specific IFN-γ (196.8±56.8 post-vaccine vs. 32.1±6.1 pre-vaccine SFC/10⁶, p=0.02) and IL-10 (79.0±7.4 vs. 33.8±5.1 SFC/10⁶, p=0.001) responses were augmented following HER2-pulsed DC1 vaccination, relative IFN-γ:IL-10 response ratios shifted from 0.95:1 (relative T_{reg}/Th2-favoring) pre-vaccination to 2.5:1 (Th1-favoring) post-vaccination (p=0.008). However, relative IFN-γ:IL-10 response ratios did not indicate a significant shift toward a Th1-favoring phenotype following T/C treatment (0.97:1) compared with treatment-naïve (0.74:1, p=0.78) HER2^{pos}-IBC patients. See, FIGS. 12A and 12B, lower horizontal bar graphs.

Longitudinal Th1 immune evaluation ≧6 months' post-vaccination was possible for nine (81.8%) patients. As shown in FIGS. 12D and 12 E, despite completion of postoperative chemotherapy following vaccination in all patients, durable anti-HER2 Th1 reactivity was observed at a median duration of 16 (range 6-60) months vs. pre-vaccination baseline: anti-HER2 responsivity (100%≧6 mo post-vaccine vs. 22.2% pre-vaccine, p=0.008), repertoire (4.0±0.4 vs. 0.3±0.2, p<0.0001), cumulative response (255.1±49.2 vs. 33.8±9.2 SFC/10⁶, p=0.006).

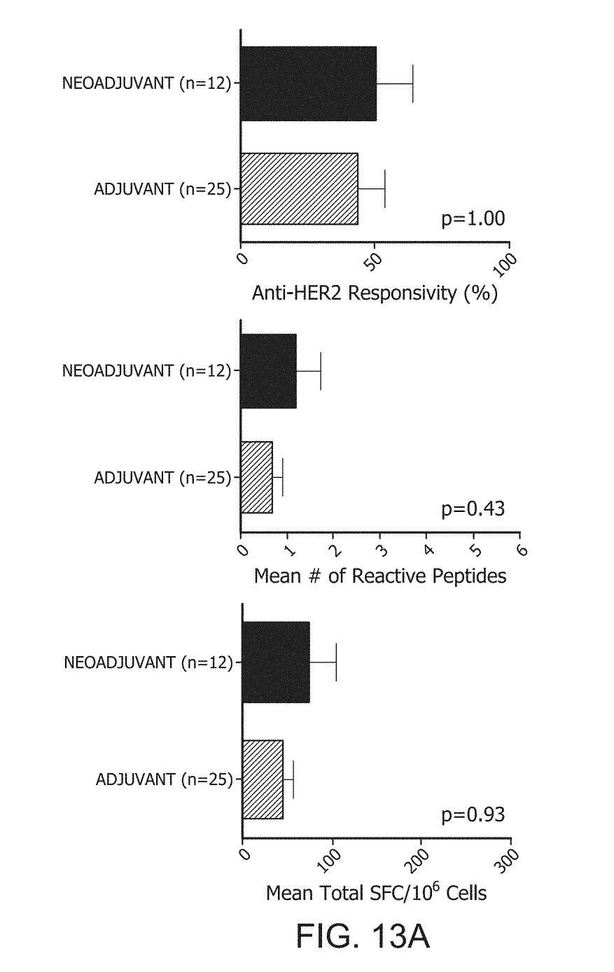

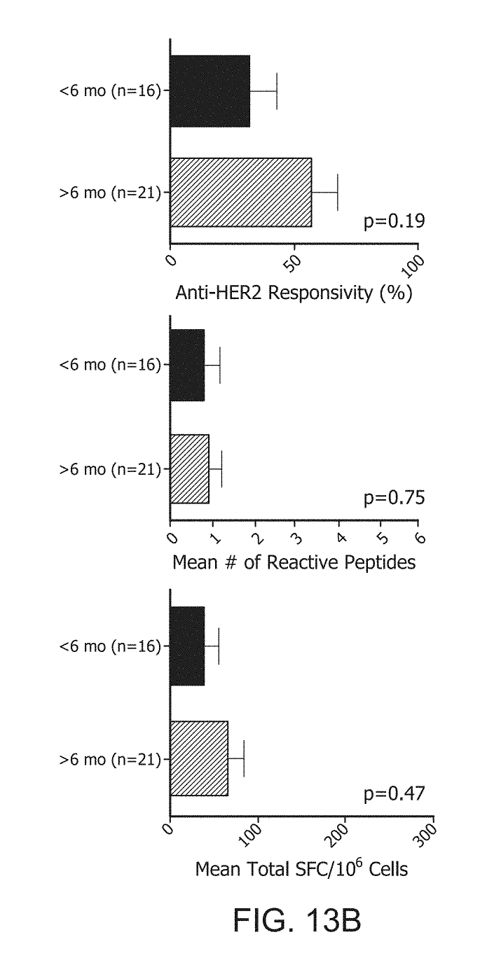

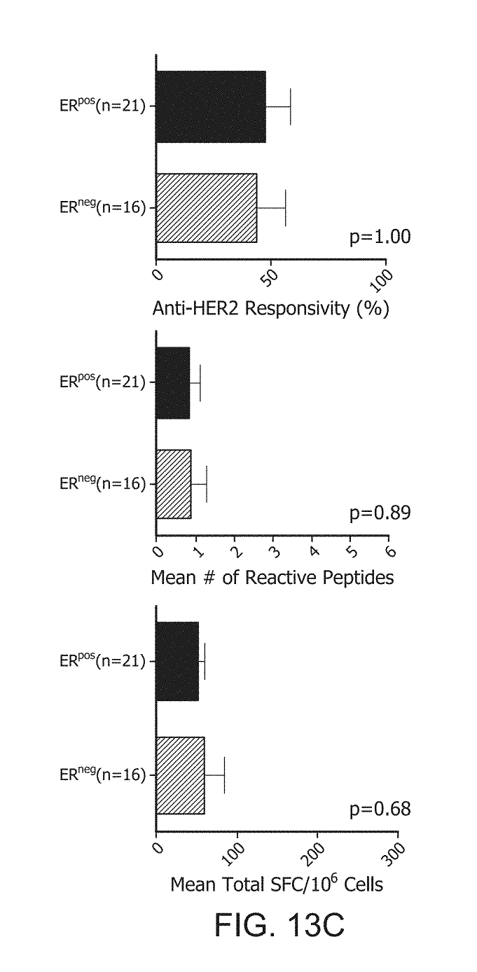

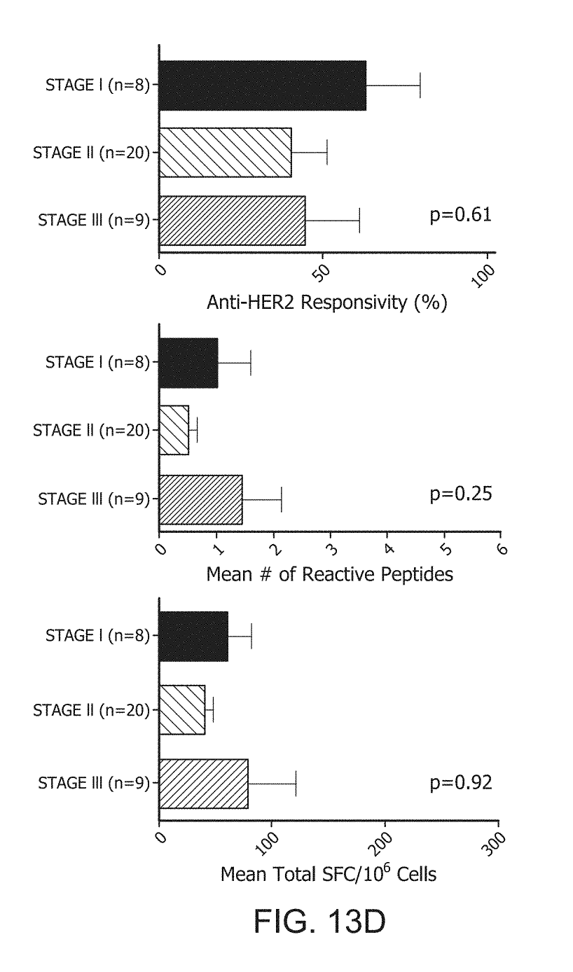

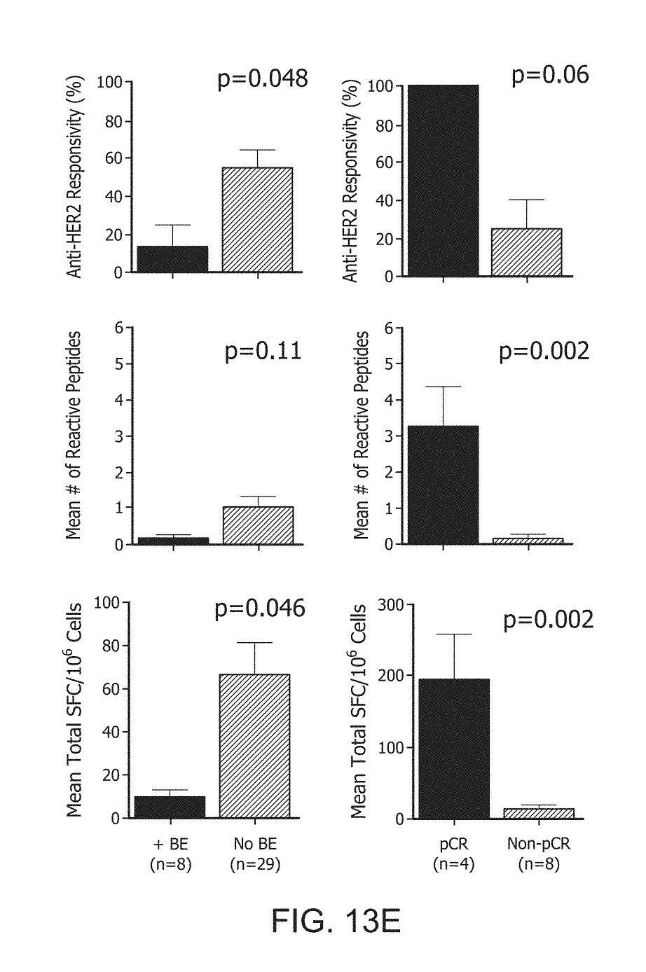

Subgroup analysis of the T/C-treated cohort was performed in order to investigate variations in Th1 reactivity by sequence of chemotherapy (neoadjuvant or adjuvant); time from completion of prescribed trastuzumab to study enrollment (< or ≧6 months); estrogen-receptor status (ER^{pos} or ER^{neg}); and pathologic stage (I-III). FIG. 13A shows chemotherapy sequence (neoadjuvant [n=12] vs. adjuvant [n=25]), FIG. 13B shows time from trastuzumab completion (<6 [n=16] vs. 26 months [n=21];), or FIG. 13C shows ER status (ER^{pos} [n=21] vs. ER^{neg} [n=16];) did not impact anti-HER2 Th1 responsivity, repertoire, or cumulative response (all p=NS). Importantly, FIG. 13D shows AJCC stage I (n=8), stage II (n=20), or stage III (n=9) T/C-treated patients did not differ by any Th1 metric, suggesting that the observed anti-HER2 Th1 deficit in HER2^{pos}-IBC was independent of disease burden. Moreover, these data collectively suggest that dominant Th1 reactivity profiles of particular subgroups are not responsible for the lack of immune restoration observed globally in T/C-treated HER2^{pos}-IBC patients.

Depressed Anti-HER2 Th1 Responses Correlate with Adverse Clinicopathologic Outcomes

To assess the translational relevance of these findings, an evaluation was made to determine if Th1 response variations in T/C-treated HER2^{pos}-IBC patients were associated with the development of subsequent breast events (“BE;” defined as any locoregional/distant recurrence). Median follow-up was 33.5 (interquartile range “IQR” 25.5-45.8) months. As shown in Table 2 below (showing demographic and clinical characteristics of HER2^{pos}-IBC patients incurring subsequent breast cancer events (defined as any locoregional or systemic recurrence) following trastuzumab and chemotherapy treatments), eight patients (21.6%) suffered BEs following T/C treatment at a median duration of 29 (IQR 16.2-36) months. FIG. 13E, left panels, show that compared with patients without BEs, BE-incurring patients had significantly depressed anti-HER2 responsivity (top) (12.5%+BE vs. 55.2% no BE; p=0.048) and cumulative responses (bottom) (9.4±3.6 vs. 66.9±14.5 SFC/10⁶; p=0.046), but not response repertoire (middle) (1.03±0.3 vs. 0.13±0.1, p=−0.11).

TABLE 2

| | Age at | | Location, | Stage at | | Time to |
|---|---|---|---|---|---|---|
| | study | | if distant | initial | Timing | recur- |
| Pt | entry | Type of | recur- | diag- | of T/C | rence |
| no. | (yrs) | recurrence | rence | nosis | receipt | (months) |
| 1 | 64 | Systemic | Bone, | 3 | Adjuvant | 26 |
| | | | brain | | | |
| 2 | 63 | Locoregional | — | 3 | Adjuvant | 31 |
| 3 | 53 | Locoregional | — | 2 | Adjuvant | 21 |
| 4 | 43 | Locoregional | — | 2 | Adjuvant | 12 |
| 5 | 49 | Locoregional, | Bone | 2 | Adjuvant | 31 |
| | | Systemic | | | | |
| 6 | 67 | Locoregional | — | 1 | Adjuvant | 102 |
| 7 | 85 | Locoregional | — | 3 | Adjuvant | 36 |
| 8 | 34 | Locoregional | — | 3 | Neo- | 14 |
| | | | | | adjuvant | |

In 12 (32.4%) T/C-treated HER2^{pos}-IBC patients receiving neoadjuvant T/C, anti-HER-2 Th1 responses were compared between pathologic complete responders (“pCR”; defined as no evidence of residual invasive BC on postoperative pathology) and non-pCR patients. The results in FIG. 13E, right panels, show pCR, achieved in 4 patients (33.3%), was associated with significantly higher anti-HER2 repertoire (3.3±1.1 vs. 0.13±0.0.13, p=0.002) (middle) and cumulative response (193.1±64.9 vs. 13.6±4.6, p=0.002) (bottom) compared with non-pCR patients; anti-HER2 responsivity (100% vs. 25%, p=0.06) (top) did not reach statistical significance.

### Discussion

The advent of checkpoint inhibitors (Topalian, S. L., et al., N. Eng. J. Med. 366:2443-54 (2012)), and use of immune-modulating strategies such as vaccines (Kantoff, P. W., et al., N. Eng. J. Med. 363:411-22 (2010)), toll-like receptor agonists, or adoptive T-cell therapies against tissue-specific epitopes (Kalos, M., et al., Sci. Transl. Med. 3(95):95ra 73 (2011) and Rosenberg, S. A., Nature Reviews Clinical Oncology 8:577-85 (2011)) have set the stage for more effective cancer immunotherapies. Most of these therapies are geared toward broad-based immune modulation. In parallel with these discoveries, genomic profiling has identified specific molecular drivers of tumorigenesis, including v-raf murine sarcoma viral oncogene homolog-B1 (“BRAF”), epidermal growth factor receptor (“EGFR”), hepatocyte growth factor receptor (“c-MET”), and HER2. While therapies targeting such “oncodrivers” achieve encouraging response rates, their success is relatively short-lived because most tumors ultimately recur or become therapy-resistant (Pohlmann, et al. and Flaherty, K. T., et al., N. Eng. J. Med. 363:809-19 (2010)). Identifying oncodriver-specific immune deficits during tumor development may provide therapeutic opportunities tailored to specific cancer subtypes. Herein described is believed to be the first study that identifies a CD4⁺ Th1 immune deficit in tumorigenesis specific to the molecular oncodriver of a defined BC phenotype, namely HER2/neu.

The decay in anti-HER2 CD4⁺ Th1 immunity commences in the premalignant DCIS phase, and becomes progressively lost in early invasive disease states. Moreover, Th1 immunity appears to be lost specifically in HER2-overexpressing phenotypes. Utilizing a broad tumorigenic continuum, it has been demonstrated herein that anti-HER2 Th1 responses in HER2^{neg}-DCIS and HER2^{neg}-IBC patients (IHC 0/1+) closely resembled those seen in HD/BD donors, and were significantly higher than Th1 responses seen in HER2^{pos}(IHC 3+ or 2+/FISH positive) DCIS and IBC patients, respectively; additionally, Th1 immunity appears to be lost in equivocal HER2-expressing (IHC 2+/FISH negative) individuals and resembled those seen in HER2^{pos}-IBC patients. Particularly, the maintenance of HER2-specific CD4⁺ immunity in HER2^{neg}-IBC patients may, in part, explain their improved clinical outcome after vaccination with HER2 peptides aimed at activating CD8⁺ T-cells. See, Benavides, L. C., et al., Clin. Can. Res. 15:2895-904 (2009).

It is somewhat surprising that HD/BDs maintained a readily identifiable population of circulating anti-HER2 Th1 cells. Since HER2 is normally a membrane constituent in branching breast ductal cells during pregnancy and lactation (Press, M, F., et al., Oncogene 5:953-62 (1990)), it is plausible that pre-existing CD4⁺ T-cell responses in HD/BDs are generated as a result of HER2-epitope presentation by antigen-presenting cells (“APCs”) within the breast. Indeed, although independent of age, race, or menopausal status, pre-existing anti-HER2 Th1 immunity in HD/BDs was higher in gravid compared with non-gravid donors; notably, the latter is a population at increased risk for BC development. Furthermore, the striking pro-apoptotic effect of HER2-specific Th1-via cytokines IFN-γ and TNF-α-in HER2^{high}, but not HER2^{low}, BC cell lines expressing IFN-γ/TNF-α receptors in vitro, imply that anti-HER2 Th1 may be instrumental in controlling or eliminating HER2-overexpressing cells during physiologic processes such as breast involution. Thus, a pre-existing anti-HER2 Th1 immunity in HDs may confer protection against tumorigenic events, while abrogation of anti-HER2 Th1 function may represent a tumor-driven mechanism to evade immune surveillance during HER2^{pos} tumorigenesis. Interestingly, recent evidence suggests that preferential death programming of circulating tumor-associated antigen (e.g., MAGE6, EphA2)-specific CD4⁺ Th1 may contribute to the immune dysfunction observed in melanoma patients with active disease (Wesa, A. K., et al., Front. Oncol. 4:266 (2014). Similar mechanisms may be involved in the loss of anti-HER2 CD4⁺ Th1 immunity observed in the present study—deciphering, and targeting, such mechanisms may be critical for the development of immune interventions aimed at primary BC prevention. These mechanisms, as well as the functional significance of anti-HER2 Th1 cells in breast homeostasis, warrant further investigation.

Although antecedent HER2-Th1 immunity was maintained in HD/BDs, HER2-reactive humoral responses were not. In the healthy breast, priming of CD4⁺ Th1 cells by APCs in a non-inflammatory setting, while contributing to homeostasis of HER2-expressing cells via IFN-γt/TNF-α secretion, may not drive antibody production. In HER2^{pos}-DCIS, however, a relative increase in HER2-reactive IgG1/IgG4 was associated with intermediate, but not absent, Th1 responses. Appearance of HER2 antigenic stimulus on evolving tumors, and its subsequent presentation by APCs to remaining Th1 cells in an inflammatory environment, may allow for transient antibody production. Ultimately, in HER2^{pos}-IBC, waning of CD4⁺ T-cell help may erode the continued production of antibodies, resulting in their eventual disappearance. This dissipation of both arms of adaptive immunity could render these patients incapable of primary tumor prevention and control.

In addition to those discussed above, the loss of anti-HER2 Th1 immunity may reflect other mechanisms—for instance, chronic T-cell exhaustion or peripheral tolerance with a contributory role for co-inhibitory signals (e.g., TIMs, PD-L1, CTLA-4, etc.), or alterations in HER2-reactive immune phenotypes. Indeed, although overall IL-10 responses are maintained across the tumorigenic continuum, HER2-specific responses functionally shift from strongly Th1-favoring (in HD/BDs) toward a relatively Th2/T_{reg}-favoring (in HER2^{pos}-IBC) phenotype when evaluated by antigen-specific IFN-γ:IL-10 ratios. The intact, albeit muted, Th1 responsivity in 7/22 (32%) HER2^{pos}-IBC patients, therefore, may reflect an ongoing balance between Th1 antitumor immune defense and tolerogenic T_{reg}/Th2 contributions²⁸ (Levings, M, K, et al., Blood 105:1162-9 (2005) during tumorigenesis.

Nonetheless, the loss of anti-HER2 Th1 immunity was not attributable to absolute increases in circulating immunosuppressive populations in HER2^{pos}-IBC patients. Although previous studies have reported higher levels of circulating T_{reg} and/or MDSCs in advanced (Stage III/IV) BC (Liyanage, U. K., et al., J. Immunol. 169:2756-61 (2002)) and other solid tumors (Zhang, B., et al., PLOS ONE 8(2): e57114 (2013), in this study, early-stage (Stage I/II) IBC patients appear to have comparable immunosuppressive profiles to HDs. The dramatic decline in anti-HER2 Th1 responses in these patients, therefore, is even more compelling. Furthermore, this decline in peripheral blood anti-HER2 IFN-γ^{pos} CD4⁺ T-cell subsets was unrelated to (i) immune sculpting, since a bias was not observed towards selective HER2 peptide reactivity with progressive tumorigenesis; or (ii) discrepantly greater CD4⁺ T-cell trafficking to invasive tumors. The latter finding should be interpreted with caution, however, since these data do not address sequestration or depletion of HER2-specific CD4⁺ TILs in the tumor microenvironment. Finally, the anti-HER2 Th1 immune depression could not be explained by generalized host-level T-cell anergy in IBC patients; however, the present study cannot completely exclude antigen-specific cellular-level anergy as a possible explanation for this phenomenon.

Importantly, this anti-HER2 Th1 depression was associated with an increased risk of locoregional or distant recurrence in T/C-treated HER2^{pos}-IBC patients. In contrast, anti-HER2 Th1 preservation correlated with pCR following neoadjuvant T/C. Taken together, these data suggest that monitoring anti-HER2 Th1 immune reactivity following HER2-directed therapies may identify vulnerable subgroups at risk of clinical or pathologic failure. Moreover, the association of an anti-HER2 Th1 deficit with unfavorable clinicopathologic outcomes warrants a search for therapeutic strategies that might reverse such an immune deficit.

Even after controlling for disease burden (i.e. pathologic stage), the depressed anti-HER2 Th1 responses in HER2^{pos}-IBC patients remained globally unaffected by surgery, radiation, chemotherapy, or HER2-targeted trastuzumab. Several studies have demonstrated the ability of trastuzumab to reduce growth and induce apoptosis in HER2^{pos} tumors (Dogan, I., et al., Mol. Cell. Biochem. 347:41-51 (2011), as well as to sensitize HER2^{pos} cells to the tumoricidal effects of cytotoxic chemotherapy (Henson, E. S., et al., Clin. Cancer Res. 12:645-53 (2006)). Despite these benefits, the use of trastuzumab did not appreciably restore HER2-specific Th1 immunity in a majority of patients, including those with Stage I disease. In addition, an almost universal resistance to these HER2-targeted therapies is observed in advanced disease states. Pohlman, et al. Additional strategies targeting HER2, therefore, are required.

One such strategy, described herein, may be autologous DC1 immunization with HER2-derived Class II peptides. Following neoadjuvant HER2-pulsed DC1 vaccination in HER2^{pos}-IBC patients (followed by surgery), durable restoration of anti-HER2 Th1 immunity was observed up to 60 months post-vaccination. Altogether, these data suggest that (i) this HER2-specific CD4⁺ Th1 immune deficit is not immunologically “fixed,” since it can be corrected with appropriate immunologic interventions; and (ii) combination of vaccination (or other immune-modulating strategies) with existing humoral-based HER2-targeted therapies may improve long-term outcomes in this disease. Indeed, in murine models, the collaboration of cellular (IFN-γ-producing CD4⁺, but not CD8⁺, T-cells (Sakai, Y., et al., Cancer Res. 64:8022-8 (2004)) and humoral HER2-directed immunity is essential for eradication of HER2^{pos} tumors (Reilly, R. T., et al., Cancer Res. 61:880-3 (2001)).

Collectively, the present findings have implications for immune monitoring and therapy selection in HER2^{pos}-BC patients. As discussed, they justify addition of anti-HER2 immunizations to standard HER2-targeted therapies in high-risk populations with HER2-driven BC; indeed, trials have been initiated testing such combinations in HER2^{pos}-IBC patients with residual disease after neoadjuvant T/C, and those with advanced disease following adjuvant therapy. Moreover, while conventional surveillance strategies (radiographic imaging, IHC/FISH profiling of breast biopsy specimens, etc.) offer only an isolated snapshot of a tumor's evolution, monitoring high-risk patients for real-time fluctuations in their anti-HER2 Th1 immunity may provide a glimpse into the natural history and immune repercussions of a tumor. Judicious incorporation of CD4⁺ Th1 immune detection protocols into future BC clinical trial design appears justified.

In summary, it is believed that herein is the first description, to our knowledge, of the progressive and specific loss of CD4⁺ Th1 immunity to a molecular oncodriver during breast tumorigenesis. Glimpses into the unfavorable clinical and pathologic outcomes associated with depressed anti-HER2 Th1 immunity imply that immune restoration with vaccination or other immune modulating strategies may be worth pursuing in these high-risk patients to mitigate tumor progression or prevent recurrence. Additional studies are warranted to determine whether anti-HER2 CD4⁺ responses are lost in other HER2^{pos} cancers (i.e. ovarian, gastric, etc.), and if there is a generalized loss in Th1 immunity to other molecular oncodrivers during tumorigenesis.

### Experimental Example 1

### Anti-HER2 CD4 Th1 Response is a Novel Immune Correlate to Pathologic Response Following Neoadjuvant Therapy in HER2-Positive Breast Cancer

In contemporary practice, patients with larger resectable tumors often benefit from neoadjuvant administration of trastuzumab and chemotherapy (T/C), with nearly 40%-60% achieving pathologic complete response (“pCR”). See, Gianni, L., et al., Lancet 375:377-84 (2010); Untch, M., et al., J. Clin. Oncol. 28:2024-31 (2010); Untch, M., et al., J. Clin. Oncol. 29:3351-7 (2011). Compared with evidence of residual disease at surgery (“<pCR”), attainment of pCR following neoadjuvant T/C is an established surrogate for decreased recurrence and improved long-term survival.

The above Reference Example demonstrated a progressive loss in anti-HER2 CD4⁺ T-helper type-1 (“Th1”) immunity across a tumorigenic continuum in HER2^{pos}-breast cancer. Of particular interest, this HER2-specific Th1 response is preserved in healthy volunteers as well as patients harboring HER2^{neg} (0-1+) invasive breast cancer (“IBC”). In HER2^{pos}-IBC patients, this anti-HER2 Th1 deficit does not appear to be impacted by standard therapies—surgical resection, radiation, or T/C treatment—but instead can be “restored” following HER2-pulsed type-1-polarized dendritic cell (DC1) vaccinations. Moreover, also shown was that depressed anti-HER2 Th1 responses predict an increased risk of subsequent recurrence in adjuvant T/C-treated patients. These observations prompted a study of whether similar depressed anti-HER2 Th1 responses are observed in another known harbinger of recurrence, namely, <pCR status following neoadjuvant T/C (Kim, M. M., et al., Ann. Oncol. 24:1999-2004 (2013)); conversely, it was hypothesized that preservation/restoration of anti-HER2 Th1 responses may be associated with pCR. Therefore, differences in anti-HER2 Th1 responses between pCR and <pCR patients were examined to identify modifiable immune correlates to pathologic response.

Anti-HER2 CD4: Th1 responses were analyzed prospectively for 87 HER2^{pos}-IBC patients (3⁺ or 2⁺/FISH-positive) and responses were compared between stage I/II HER2^{pos}-IBC (n=22) and stage I-III T/C-treated HER2^{pos}-IBC patients (n=65). In the T/C-treated cohort—anti-HER2 Th1 responses were generated following completion of adjuvant trastuzumab—responses were stratified by timing of chemotherapy (i.e., neoadjuvant vs. adjuvant), and further sub-stratified by pCR and <pCR status within the neoadjuvant cohort. pCR was defined as absence of residual invasive cancer on pathologic examination of the resected breast specimen and sampled lymph nodes (i.e., ypT0/Tis ypN0).

Four patients in the <pCR cohort were recruited to join an adjuvant HER2-pulsed type-1-polarized DC (DC1) vaccination trial (NCT02061423); anti-HER2 Th1 responses in these patients were analyzed pre- and post-immunization.

### Methods

As described in the Reference Example, circulating anti-HER2 CD4⁺ Th1 responses were examined in unexpanded PBMCs pulsed ex vivo with six HER2-derived class II peptides (peptide 42-56, peptide 98-114, peptide 328-345, peptide 776-790, peptide 927-941, and peptide 1166-1180) (SEQ ID NOS: 1-6), by measuring IFN-γ production via enzyme-linked immunosorbent spot (ELISPOT) assays. ELISPOT was performed as described in the Reference Example. PBMCs from HLA-A2.1^{pos} donors were stimulated with two HER2-derived class I peptides: peptide 369-377 (SEQ ID NO: 7) and peptide 689-697 (SEQ ID NO: 8) with PMA (50 ng/ml) and ionomycin (1 μg/ml; Sigma-Aldrich) serving as positive control.

An empiric method of determining antigen-specific response was employed. A positive response to an individual HER2 peptide was defined as: (1) threshold minimum of 20 SFC/2×105 cells in experimental wells after subtracting unstimulated background; and (2) ≧two-fold increase of antigen-specific SFCs over background. Th1 response metrics were anti-HER2 responsivity, number of reactive peptides (repertoire), and cumulative response across 6 peptides (SFC/106 cells) as described in the Reference Example. Th1 responses of <pCR patients (n=4) receiving adjuvant HER2-pulsed type-1-polarized dendritic cell (DC1) vaccination were analyzed pre-/post-immunization.

### Results

The study comprised 87 patients. Depressed anti-HER2 Th1 responses in treatment-naïve HER2^{pos}-IBC patients (n=22) did not improve globally after T/C treatment (n=65). Compared with adjuvant-T/C, neoadjuvant-T/C (61.5%) was associated with higher Th1 repertoire (1.5 vs. 0.8, p=0.048). While pCR (n=16) and <pCR (n=24) patients did not differ in demographic/clinical characteristics, pCR patients were more likely to have ER^{neg} tumors. pCR patients demonstrated dramatically higher anti-HER2 responsivity (94% vs. 33%, p=0.0002), repertoire (3.3 vs. 0.3, p<0.0001), and cumulative response (148.2 vs. 22.4, p<0.0001) compared with <pCR patients. This disparity was mediated by CD4⁺ T-bet⁺IFN-γ⁺ phenotypes, and not attributable to <pCR patients' immune incompetence, host-level T-cell anergy, or increased immunosuppressive populations. In four<pCR patients, Th1 repertoire (3.7 vs. 0.5, p=0.014) and cumulative responses (192.3 vs. 33.9, p=0.014) improved significantly following HER2-pulsed DC1 vaccination.

### Conclusion

Anti-HER2 Th1 response is a novel immune correlate to pathologic response following neoadjuvant-T/C. In <pCR patients HER-2 expressing patients receiving neoadjuvant therapy, depressed Th1 responses can be restored with HER2-Th1 immune interventions and may improve pCR or recurrence rates.

Thus, addition of HER2-targeted Th1 immune interventions to neoadjuvant T/C regimens and/or in the adjuvant setting for high-risk<pCR subgroups may be justified. Moreover, in light of the demonstration in the Reference Example that depressed anti-HER2 Th1 immunity correlates with subsequent recurrence in adjuvant T/C-treated patients, monitoring high-risk<pCR patients for real-time fluctuations in anti-HER2 Th1 immunity may complement existing radiographic surveillance, and help identify critical windows in which to intervene therapeutically.

In summary, this believed to be the first description of a critical association between anti-HER2 CD4⁺ Th1 immunity and pCR following neoadjuvant T/C in HER2^{pos}-IBC patients. Although causality cannot be confirmed, the dramatic IFN-γ⁺ anti-HER2 Th1 deficit observed in <pCR patients following neoadjuvant TIC raises the possibility that immune rescue with HER2-Th1 interventions may complement standard HER2-targeted strategies in improving outcomes in these high-risk patients.

### Experimental Example 2

### Depressed Anti-HER2 CD4+ T-Helper Type 1 Response is Associated with Recurrence in Completely Treated HER2-Positive Breast Cancer Patients—New Role for Immune Monitoring

As shown herein in the Reference Example using a prospective cohort, there is a progressive loss in anti-HER2 CD4⁺ T-helper type-1 (“Th1”) immunity across a tumorigenesis continuum in HER2^{pos}-BC, extending from healthy donors, through HER2^{pos}-DCIS, and ultimately to HER2^{pos}-invasive BC patients. See, Datta, J., et al., Oncolmmunology 4:8 e1022301 (2015) DOI:10. 1080/2162402X.2015. 1022301 (“Datta, J., et al. OncoImmunology). Additionally, in Experimental Example 1 herein anti-HER2 Th1 response is shown to be a novel immune correlate to pathologic response following neoadjuvant trastuzumab/chemotherapy (“T+C”) in HER2^{pos}-BC, whereas depressed anti-HER2 Th1 immunity correlated with residual disease at surgery—an unfavorable clinicopathologic outcome. See, Datta, J., et al., Breast Cancer Res. 17(1):71 (2015) (“Datta, et al. Breast Cancer Res.”).

In light of these observations, it was hypothesized that anti-HER2 Th1 immunity may also be associated with locoregional and/or distant recurrence in completely treated HER2^{pos}-BC patients. In an exploratory cohort, an examination was made of differences in circulating anti-HER2 Th1 immunity between recurrent and disease-free HER2^{pos}-BC patients in order to identify immune correlates to disease recurrence.

### Methods

### Study Design

After approval by the Institutional Review Board of the University of Pennsylvania, 95 HER2^{pos}-BC patients were recruited in a non-biased fashion after informed consent was obtained. Eligible patients had histologically-confirmed invasive breast cancer (“IBC”), HER2/neu overexpression (IHC 3+ or 2+/FISH-positive), and were not receiving immunosuppressive medications. Anti-HER2 CD4⁺ Th1 responses in treatment-naïve (i.e., no definitive therapy at enrollment) stage I-III HER2^{pos}-IBC patients (n=22) were compared with Th1 responses in stage I-IV HER2^{pos}-IBC patients who had completed T+C treatment (n=73; i.e., either neoadjuvant or adjuvant T+C plus definitive surgery). In T+C-treated patients, analyses were stratified by recurrence status.

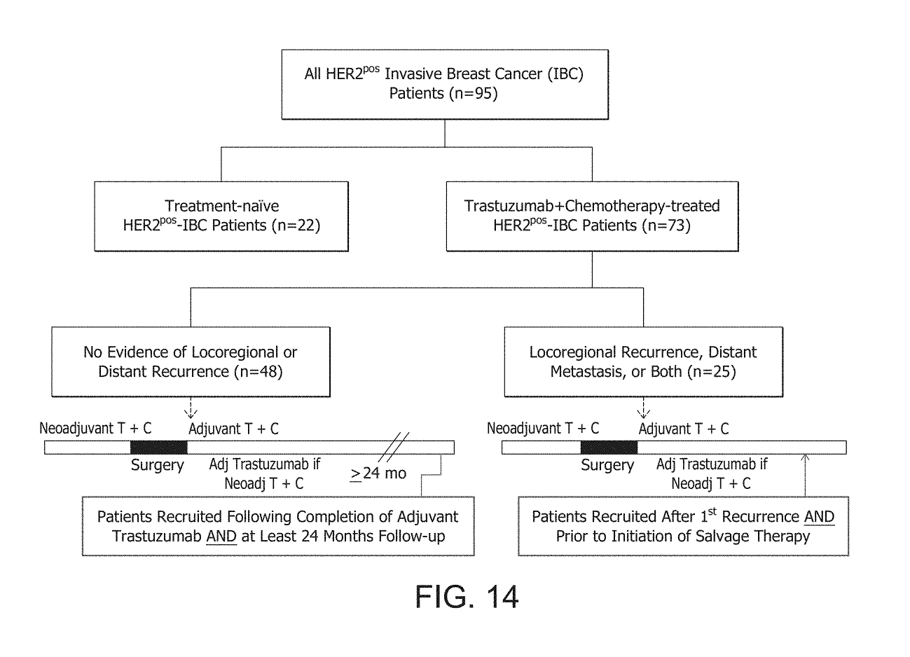

HER2^{pos}-confirmed recurrences were defined as any locoregional (in-breast/chest wall recurrence, axillary failure) or distant (lung, bone, brain metastasis, etc.) breast event, or both. All patients incurring recurrence were enrolled prior to initiation of additional chemotherapy, HER2-targeted therapy, or experimental (e.g., HER2-pulsed dendritic cell vaccination) protocols. Non-recurrent patients were eligible for analysis only if they were disease-free at a minimum follow-up of 24 months. FIG. 14 is a flow diagram of the study populations for this Experimental Example.

### Scheduling and Dosing of Trastuzumab and Chemotherapy Regimens

T+C-treated patients received one of the following regimens, either preoperatively or post-operatively: (1) AC/TH: Adriamycin/Cytoxan (every 2 weeks for 4 cycles) followed by Taxol and concurrent trastuzumab (weekly for 12 weeks); (2) TCH: Taxotere/carboplatin with concurrent trastuzumab (every 3 weeks for 6 cycles), or (3) TC-H: Taxotere/Cytoxan with concurrent trastuzumab (every 3 weeks for 4 cycles). All patients received additional trastuzumab alone (every 3 weeks) to complete a full year of therapy.

### Immune Response Detection

As described above in the Reference Example, peripheral blood anti-HER2 CD4⁺ Th1 responses were examined in unexpanded PBMCs pulsed ex vivo with six HER2-derived MHC class II— peptides (42-56, 98-114, 328-345, 776-790, 927-941, 1166-1180), by measuring IFN-γ production via ELISPOT; see also, Datta, J., et al., Oncolmmunology; Datta, J., et al., Breast Cancer Res.; and Koski, et al. Briefly, between 25 and 30 mL of whole blood was collected from each study participant in 5 heparinized collection tubes (BD Bioscience). Shortly after phlebotomy (<4-6 hours), PBMCs were isolated using density gradient centrifugation (i.e., Ficoll-Paque method; Fisher Scientific) according to manufacturer's instructions, and cryopreserved at 10×10⁶ cells/mL in 10% DMSO in human serum at −80° C. All PBMCs were utilized within 4 weeks of cryopreservation. Viability upon thaw was between 60 and 80%. PVDF membrane plates (Mabtech Inc.) were coated overnight with anti-IFN-γ capture antibody. Cryopreserved PBMCs were thawed into pre-warned DMEM+5% human serum, and prepared at 1×10⁶ PBMCs/mL in said medium. After plates were washed and blocked, PBMCs were plated in triplicate (2×10⁵ cells/well), and incubated at 37° C. for 24-48 hours with either HER2 peptide(s) (4 μg; Genscript, Piscataway, N.J.); media alone (unstimulated control); or positive control (anti-human CD3/CD28 antibodies [0.5 μg/mL; BD Pharmingen]).

In evaluable patients. Th1 responses to 1:100-diluted recall stimuli Candida albicans (Allermed Laboratories) and tetanus toxoid (Santa Cruz Biotechnology) were examined as described in the Reference Example. HER2-specific IL-4 and IL-10 production, surrogates for T-helper type-2 (Th2) and regulatory T-cell (T_{reg}) function, was measured by ELISPOT as described by Datta, J., et al., Breast Cancer Res.

Anti-HER2 Th1 reactivity was determined using empiric methodology as described in the Reference Example. A positive/reactive response to an individual HER2 peptide was defined as ≧20 spot-forming cells [SFC]/2×10⁵ PBMCs in experimental wells after subtracting unstimulated background. Three metrics of anti-HER2 Th1 response were measured: (a) responsivity (proportion of patients responding to at least one of 6 peptides), (b) repertoire (mean number of reactive peptides), and (c) cumulative response across 6 peptides (SFC/10⁶ cells).

### Flow Cytometry

PBMCs were suspended in FACS buffer (PBS+1% FCS+0.01% azide). PE/FITC/Cy5-conjugated mouse anti-human CD3, CD4, CD25, T-bet, GATA-3, IFN-γ, or subclass-matched controls (BD Bioscience) were used to determine relative PBMC immunophenotype as described by Datta, J., et al., Breast Cancer Res. Intracellular staining with anti-FoxP3 using FoxP3 fixation/permeabilization kit (Biolegend) was performed according to manufacturer's instructions. Analysis was performed using BD LSR-II cytometer, and datasets analyzed using CellQuest Pro software.

Functional Contribution of Th1 Vs. Th2 Subtypes

PBMCs were resuspended at 1.2×10⁶ cells/mL in DMEM+5% human serum in 24-well plates, and pulsed with HER2-class II peptide mix (24 μg/mL). Unstimulated and anti-CD3/CD28 antibody-pulsed PBMCs from each donor served as negative and positive control, respectively. Following incubation for 6 hours at 37° C., protein transport inhibitor Brefeldin-A (Sigma Aldrich; 10 μg/ml) was added to each sample, and incubated overnight. Following washing, cells were stained with anti-human CD4 for 30 min at room temperature. Cells were subsequently washed twice, fixed and permeabilized using Foxp3 fixation/permeabilization kit (Biolegend) according to manufacturer's instructions, and stained with anti-T-bet, anti-GATA-3 and anti-IFN-γ (Biolegend) for 30 min. After incubation, cells were washed and analyzed on a BD LSR-II cytometer.

### Statistical Analysts

Descriptive statistics summarized distributions of patient characteristics and immune response variables. Comparisons between non-recurrent and recurrent HER2^{pos}-IBC cohorts were performed as indicated: (a) 2-group/univariate testing—unpaired Student's t-test (parametric continuous), Wilcoxon rank-sum test (non-parametric continuous), and t-tests (categorical); and (b) >2-group testing—one-way ANOVA with post-hoc Bonferroni testing. Variables with p<0.1 on univariate testing were entered into a forward, stepwise multivariable logistic regression model (p<0.05 for entry) to determine independent correlates to recurrence (binary outcome yes/no).

Univariate disease-free survival (“DFS”) estimates were examined by Kaplan-Meier methodology, stratifying by anti-HER2 Th1 responsivity and other covariates. Observations of non-recurrent patients (minimum 24-month follow-up) were censored at last known follow-up. To analyze the instantaneous hazard of all variables and control for varied follow-up, Cox proportional hazards modeling was performed. The assumptions of the Cox model were assessed, including interactions and proportionality of hazards over time. P<0.05 was considered statistically significant. All tests were two-sided. Analyses were performed using SPSS v22 (IBM Corp. Armonk, N.Y.).

### Results

### Patient Characteristics

Demographic and tumor-related characteristics of the overall cohort (n=95) are detailed in Table 3 below. In T+C-treated patients (n=73), median age was 49 (range, 24-85) years, and a majority were white (87.5%). A majority of patients had estrogen/progesterone receptor-positive (ER/PR^{pos}) tumors (58.9%), and presented with locally advanced/node-positive disease (clinical stage I: 9.6%, II: 47.9%, III: 42.5%). Neoadjuvant T+C was administered in 37 (50.7%) patients. Simple or modified radical mastectomy was performed most frequently (53.4%), and Adriamycin/Cytoxan/Taxol/Herceptin was the commonly utilized T+C regimen (57.5%).

Twenty-five (34.2%) patients incurred either locoregional recurrence, distant recurrence, or both as shown in Table 4 below; Th1 responses in a majority (n=21; 84%) of these patients were determined at the time of diagnosis of recurrence.

TABLE 4
Demographic, tumor-related and treatment characteristics of HER2-overexpressing breast cancer
patients incurring recurrence following trastuzumab and chemotherapy (T + C).

| | | Stage at | Timing | | | Initial | | | Time to | |
|---|---|---|---|---|---|---|---|---|---|---|
| Pt | Age | initial | of T + C | ER/ | Initial | HER2 | Type of | Location, | recurrence | Salvage therapy |
| no. | (yrs) | diagnosis | receipt | PR | operation | agent | recurrence | if distant | (months) | following recurrence |
| 1 | 48 | 3 | Neoadj | + | L MRM, | Trast | Distant | Bone | 22 | Chemo, Lapatinib |
| | | | | | R simple | | | | | |
| 2 | 51 | 2 | Adj | + | L BCS | Trast | Locoregional | — | 16 | Trastuzumab, Al |
| 3 | 24 | 2 | Neoadj | − | R BCS | Trast | Locoregional | — | 49 | Chemo, Trastuzumab |
| 4 | 52 | 2 | Neoadj | − | L BCS | Trast | Locoregional, | Lung | 16 | Chemo, Trastuzumab |
| | | | | | | | Distant | | | |
| 5 | 35 | 3 | Neoadj | − | R MRM | Trast | Distant | Lung, | 27 | Chemo, Trastuzumab, |
| | | | | | | | | liver, | | Pertuzumab |
| | | | | | | | | bone | | |
| 6 | 58 | 2 | Neoadj | + | L MRM | Trast | Distant | Lung, | 43 | Fulvestrant, Chemo, |
| | | | | | | | | Bone | | Trastuzumab, |
| | | | | | | | | | | Pertuzumab |
| 7 | 80 | 2 | Neoadj | + | Bilateral | Trast | Distant | Bone | 48 | Trastuzumab, |
| | | | | | BCS | | | | | Pertuzumab |
| 8 | 39 | 2 | Neoad | + | L BCS + | Trast | Locoregional, | Lung | 42 | Chemo, Trastuzumab, |
| | | | | | ALND | | Distant | | | Pertuzumab |
| 9 | 47 | 3 | Adj | − | R BCS | Tract | Locoregional | — | 64 | Chemo, Trastuzumab |
| 10 | 41 | 3 | Adj | + | L BCS + | Trast | Locoregional | — | 113 | Chemo, Trastuzumab |
| | | | | | ALND | | | | | |
| 11 | 52 | 1 | Adj | + | R BCS + | Trast | Locoregional | — | 82 | Chemo, Al, |
| | | | | | ALND | | | | | Trastuzumab |
| 12 | 30 | 2 | Adj | + | L BCS | Trast | Locoregional | — | 43 | HER2-DC1 vaccine |
| 13 | 50 | 3 | Adj | − | R BCS + | Trast | Locoregional | — | 42 | Resection alone |
| | | | | | ALND | | | | | |
| 14 | 55 | 2 | Adj | + | R MRM | Trast | Locoregional | — | 26 | Chemo, Trastuzumab, |
| | | | | | | | | | | Pertuzumab, Lapatinib |
| 15 | 49 | 3 | Adj | − | L MRM, | Trast, | Locoregional | — | 13 | Chemo, Lapatinib, |
| | | | | | R simple | Lap | | | | TDM-1 |
| 16 | 54 | 3 | Adj | + | R MRM | Trast, | Locoregional | — | 19 | Resection, HER2-DC1 |
| | | | | | | TDM-1 | | | | vaccine |
| 17 | 42 | 2 | Adj | + | R MRM, | Trast, | Distant | Lung | 51 | Chemo, Trastuzumab, |
| | | | | | L simple | TDM-1 | | | | HER2-DC1 vaccine |
| 18 | 45 | 3 | Adj | − | L MRM | Trast, | Distant | Lung, | 60 | Al, Trastuzumab, |
| | | | | | | Lap | | Brain | | Pertuzumab, HER2- |
| | | | | | | | | | | DC1 vaccine |
| 19 | 34 | 1 | Adj | + | L BCS | Trast | Locoregional | — | 47 | Resection + HER2- |
| | | | | | | | | | | DC1 vaccine |
| 20 | 41 | 2 | Adj | − | MRM | Trast, | Distant | Liver | 16 | Chemo, Trastuzumab, |
| | | | | | | Lap | | | | Lapatinib, Kadcycla, |
| | | | | | | | | | | CDK 4/6 inhibitor |
| 21 | 42 | 0 | Adj | + | L MRM | Trast | Locoregional, | Bone, | 23 | Al, Trastuzumab, |
| | | | | | | | Distant | Brain | | TDM-1 |
| 22 | 51 | 3 | Adj | + | L MRM, | Trast | Distant | Lung, | 58 | Chemo, Al, |
| | | | | | R BCS | | | Bone | | Trastuzumab, |
| | | | | | | | | | | Lapatinib, Peituzumab |
| 23 | 54 | 3 | Adj | + | L BCS + | Trast | Distant | Bone | 53 | Chemo, Trastuzumab, |
| | | | | | ALND | | | | | Pertuzumab |
| 24 | 44 | 2 | Adj | + | R MRM | Trast | Locoregional, | Lung | 10 | Chemo, Trastuzumab, |
| | | | | | | | Distant | | | Pertuzumab, TDM-1 |
| 25 | 62 | 2 | Adj | + | R BCS | Trast | Distant | Liver, | 46 | Chemo, Trastuzumab, |
| | | | | | | | | Lung | | Pertuzumab |

Abbreviations: T+C: trastuzumab and chemotherapy; ER: estrogen receptor, PR: progesterone receptor; Neoadj: Neoadjuvant; Adj: Adjuvant; MRM: Modified radical mastectomy; Simple: Simple mastectomy; BCS: Breast conservation surgery; ALND: Axillary Lymph Node Dissection; Trast: Trastuzumab; AI: Aromatase Inhibitor DC1: Type 1-polarized dendritic cell vaccine.
Anti-HER2 CD4⁺ Th1 Responses are Depressed in HER2^{pos}-IBC Patients Incurring Recurrence

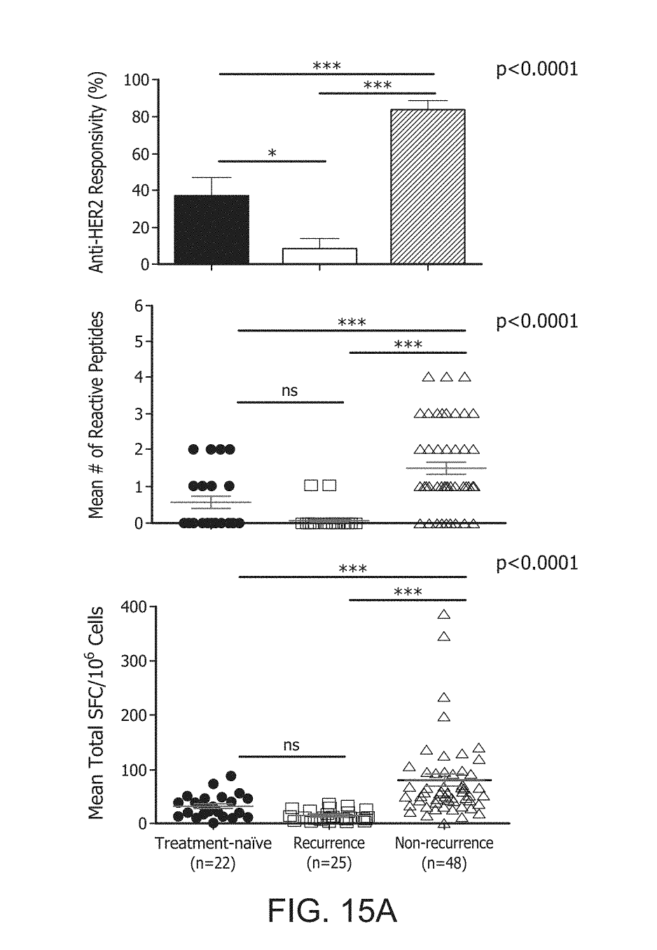

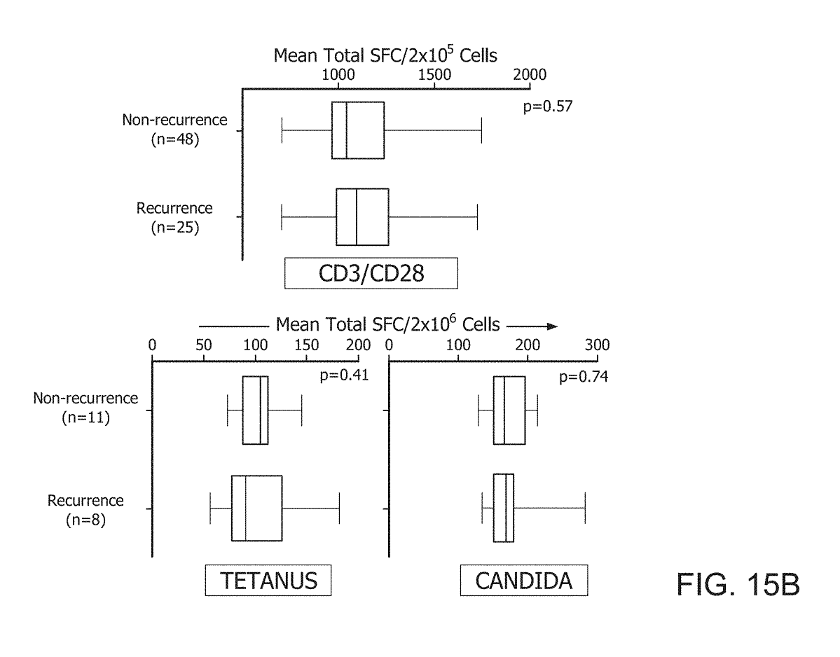

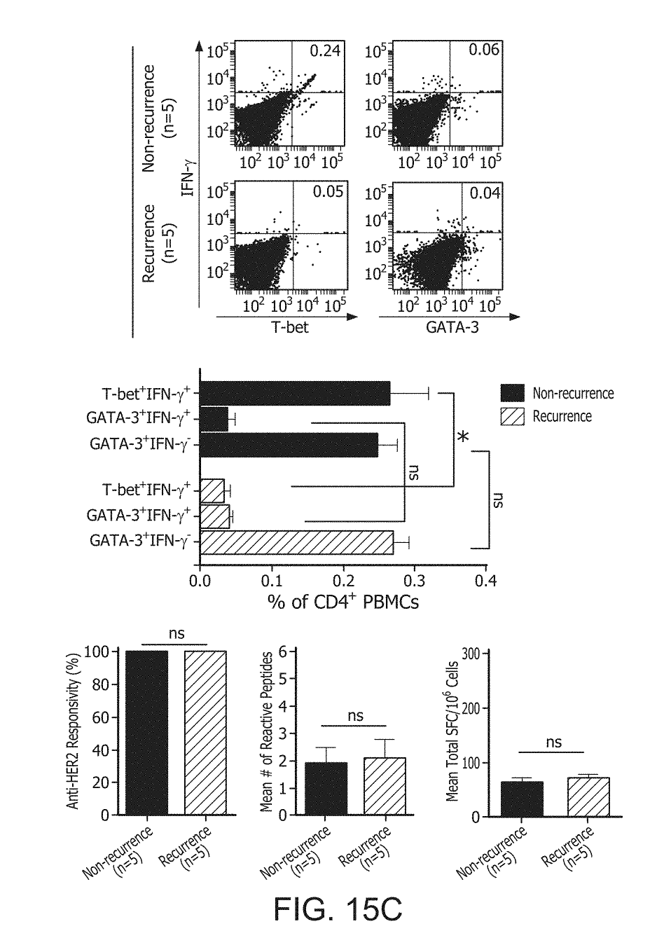

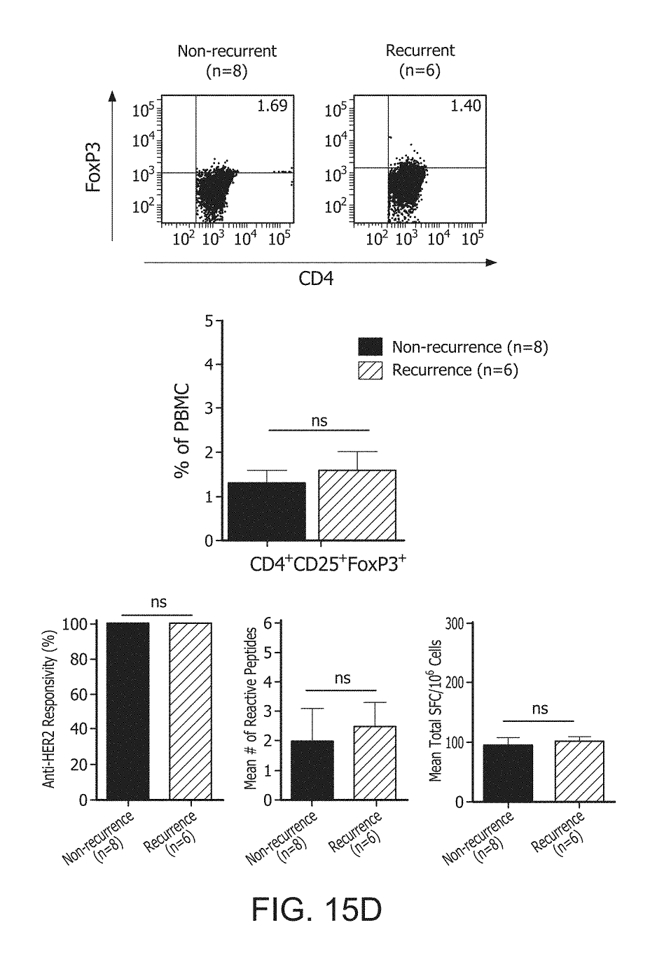

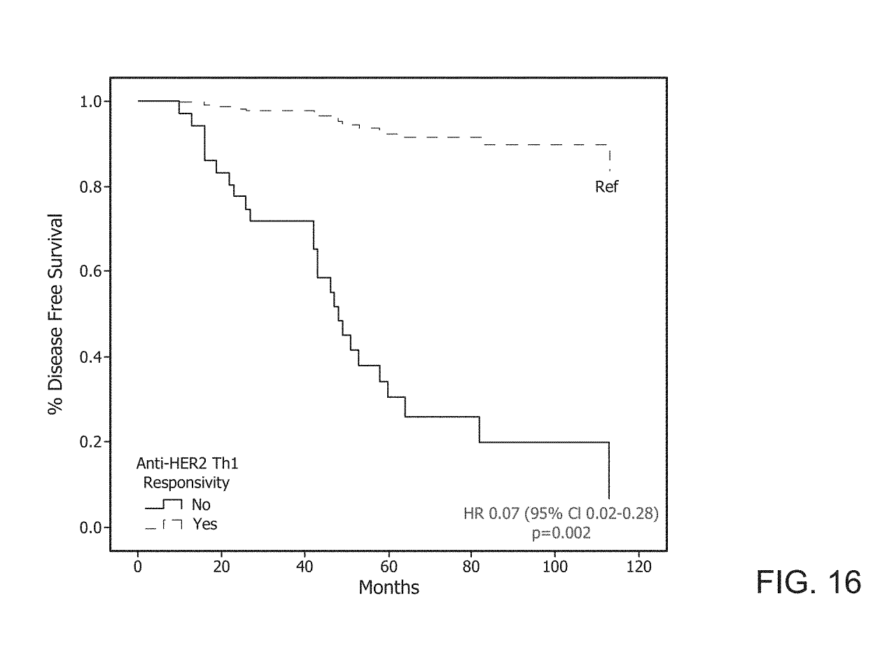

In FIG. 15A anti-HER2 Th1 responsivity, repertoire, and cumulative responses were compared between treatment-naïve (n=22), T+C-treated non-recurrent (n=48), and T+C-treated recurrent (n=25) patients. Using anti-HER2 Th1 responses from treatment-naïve patients as an immunologic “baseline,” significantly depressed anti-HER2 Th1 responsivity (treatment-naïve: 36.4% vs. recurrence: 8.0% vs. non-recurrence: 83.3%, P<0.0001) (top), repertoire (0.6±0.2 vs. 0.1±0.1 vs. 1.5±0.2, P<0.0001) (middle), and cumulative response (32.8±4.7 vs. 14.8±2.0 vs. 80.2±11.0, P<0.0001) (bottom) were observed in HER2^{pos}-IBC patients incurring recurrence compared with patients without recurrence, respectively.

In FIG. 15B IFN-γγ responses to anti-CD3/anti-CD28 stimulation (P=0.57), tetanus toxoid (P=0.41), and Candida (P=0.74) did not differ significantly between non-recurrence and recurrence cohorts, respectively, suggesting that the observed anti-HER2 Th1 disparity is not attributable to immune incompetence or host-level T-cell anergy in patients incurring recurrence.

### Depressed Anti-HER2 T-Cell Responses in Recurrent Patients is Attributable to Th1, but not Th2 or T_{reg}, Phenotypes

FIG. 15C shows HER2-stimulated PBMCs that were assessed for co-expression of T-bet (Th1 transcription factor (see, Szabo, S. J., et al., Science 295(5553):338-342 (2002)) or GATA-3 (Th2 transcription factor (see, Zheng, W., et al., Cell 89(4):587-596 (1997)), and intracellular IFN-γ by flow cytometry (top). Significantly decreased proportions of HER2-specific CD4⁺ T-bet⁺IFN-γ⁺ (0.25±0.1% vs. 0.02±0.01%, p=0.039), but not CD4⁺GATA-3⁺IFN-γ⁺ (or CD4⁺GATA-3⁺IFN-γ⁻, phenotypes were observed in the recurrence compared with non-recurrence cohort, respectively (middle). HER2-stimulated IL-4⁺ responsivity (P=1.00), repertoire (P=0.84), and cumulative response (P=0.46)—measures of Th2 function—did not differ between non-recurrence and recurrence cohorts (bottom).

In addition, FIG. 15D shows the absolute proportion of circulating T_{reg} (i.e., CD4⁺CD25⁺Foxp3⁺) phenotypes (1.3±0.3 vs. 1.6±0.4, P=0.61) did not differ between non-recurrence and recurrence cohorts, respectively; HER2-stimulated IL-10⁺ responsivity (P=1.00), repertoire (P=0.74), and cumulative response (P=0.66)—measures of T_{reg} function—were similar between the two cohorts.

Anti-HER2 Th1 Responsivity is Independently Associated with Disease-Free Survival

Independence of the association between anti-HER2 Th1 response and recurrence was examined by controlling for confounding from relevant demographic/clinicopathologic characteristics. Upon univariate testing, recurrence and non-recurrence cohorts did not differ by age, menopausal status, race, body mass index, comorbidity, clinical stage, hormone receptor status, presence of LVI, nuclear grade, or need for mastectomy. However, recurrence was more frequent among patients receiving adjuvant T+C (P=0.021) and those with residual disease following neoadjuvant therapy (P=0.006). When controlling for these associated variables via multivariable regression, anti-HER2 Th1 responsivity (P<0.0001), but not sequence of T+C therapy (P=0.304, remained independently associated with recurrence as seen in Table 5 below

TABLE 5
Univariate comparison of demographic and tumor-related characteristics between HER2^{pos}-IBC patients
with or without disease recurrence. Variables with p < 0.10 were entered into a multivariable logistic
regression to identify independent correlates to disease recurrence

| | Non-recurrence | Recurrence | | | |
|---|---|---|---|---|---|
| | n (%) or median | n (%) or median | Univariate | | Multivariable |
| Characteristic | (IQR) | (IQR) | p-value | OR (95% CI) | p-value |
| Overall population | 48 (65.8) | 25 (34.2) | — | — | — |
| Age | | | | | |
| Median (years) | 49.5 (39-57) | 48.0 (41-53) | 0.518 | | |
| BMI | | | | | |
| Median (kg/m²) | 27 (25-32) | 27.3 (24.2-31.0) | 0.506 | | |
| Race | | | | | |
| White | 39 (81.3) | 18 (72.0) | 0.365 | | |
| Black/Asian/Hispanic | 9 (18.8) | 7 (28.0) | | | |
| Charison comorbidity index | | | | | |
| 0 | 27 (56.3) | 12 (48.0) | 0.532 | | |
| ≧1 | 21 (43.7) | 13 (52.0) | | | |
| Menopausal status | | | | | |
| Pre-menopausal | 24 (50.0) | 13 (52.0) | 0.871 | | |
| Post-menopausal | 24 (50.0) | 12 (48.0) | | | |
| AJCC clinical stage^{‡} | | | | | |
| I | 5 (10.4) | 2 (8.0) | 0.941 | | |
| II | 23 (47.9) | 12 (48.0) | | | |
| III | 20 (41.7) | 11 (44.0) | | | |
| ER/PR status^{‡} | | | | | |
| Negative | 22 (45.8) | 8 (32.0) | 0.254 | | |
| Positive | 26 (54.2) | 17 (68.0) | | | |
| Lymphovascular invasion* | | | | | |
| Absent | 14 (34.1) | 11 (55.0) | 0.120 | | |
| Present | 27 (65.9) | 9 (45.0) | | | |
| Nuclear grade | | | | | |
| Low/intermediate | 15 (31.3) | 11 (44.0) | 0.280 | | |
| High | 33 (68.8) | 14 (56.0) | | | |
| Chemotherapy sequence | | | | | |
| Neoadjuvant T + C | 29 (60.4) | 8 (32.0) | 0.021 | 2.11 (0.51-8.79) | 0.304 |
| Adjuvant T + C | 19 (39.6) | 17 (68.0) | | | |
| Pathologic response (if | | | | | |
| neoadjuvant) ** | | | | | |
| Incomplete response | 13 (44.8) | 8 (100.0) | 0.006 | — | — |
| Complete response | 16 (55.2) | 0 (0.0) | | | |
| Operative approach^{‡} | | | | | |
| BCS + XRT | 23 (47.9) | 11 (44.0) | 0.750 | | |
| Mastectomy ± XRT | 25 (52.1) | 14 (56.0) | | | |
| Anti-HER2 Th1 responsivity | | | | | |
| Non-responsive | 8 (16.7) | 23 (92.0) | <0.0001 | 0.02 (0.00-0.10) | <0.0001 |
| Responsive | 40 (83.3) | 2 (8.0) | | | |

** Not included in multivariable logistic regression analysis since variable only known in patients receiving neoadjuvant T + C.
^{‡}Included in multivariable logistic regression analysis to determine independent correlates to recurrence.
Abbreviations:
BMI: Body mass index;
AJCC: American Joint Committee on Cancer;
ER: estrogen receptor;
PR: progesterone receptor;
IQR: interquartile range;
AC/TH: Adriamycin/Cytoxan/Taxol/Herceptin;
BCS: Breast-conserving surgery;
XRT: radiotherapy

FIG. 16 shows that when stratifying the analytic cohort by anti-HER2 Th1 responsivity at median follow-up of 44 (IQR 31) months, Th1-non-responsive patients demonstrated a significantly worse disease free survival (“DFS”) (median 47 vs. 113 months, P<0.0001) compared with Th1-responsive patients; this association was corroborated by Cox modeling—hazard ratio for Th1 non-responsive cohort: 15.2, 95% CI 3.5-66.7 (P<0.001).

### Discussion

In this analysis, circulating anti-HER2 CD4⁺ Th1 response is shown to be a novel immune correlate to breast cancer recurrence in completely treated HER2^{pos}-IBC patients. While not attributable to immune incompetence or host-level T-cell anergy, depressed anti-HER2 T-cell responses in patients incurring recurrence were driven predominantly by Th1, but not Th2 or T_{reg}, phenotypes. When controlling for relevant demographic/clinicopathologic factors, anti-HER2 Th1 responsivity was independently associated with recurrence, on risk-adjusted analysis, Th1-non-responsive patients demonstrated significantly worse DFS compared with Th1-responsive patients. It appears this is the first demonstration of an association between disease recurrence and a host-level immune correlate specific to a known oncodriver in breast tumorigenesis—namely HER2/neu.

These data are especially intriguing in the light of recent evidence suggesting that benefit from trastuzumab-both in the adjuvant (Perez, E. A., et al., J. Clin. Oncol. 33(7):701-708 (2015) DOI: 10.1200/JCO.2014.57.6298) (“Perez, et al.”) or neoadjuvant (Gianni, L., et al., Cancer Res. 72 (24 Supplement):S6-7 (2012)) setting—may be restricted to a cohort of immune-enriched tumors, with Th1 genes IFN-γ/TNF-α imparting a particularly dominant role. See, Perez, et al. Taken together, it appears that absent tumor-level Th1 gene expression as well as deficient circulating anti-HER2 Th1 immunity may presage failure of HER2-targeted therapy. These observations may be explained in part by in vitro evidence indicating that Th1 cytokines IFN-γ/TNF-α are critically necessary in facilitating HER2-specific CD8⁺ T-cell-mediated targeting of trastuzumab-treated HER2-overexpressing breast cancers. See, Datta, J., et al., Yale J. Biol. Med. 87(4):491-518 (2014); Datta, J., et al., Front. Immunol. 6:271 (2015); and Datta, J., et al., Cancer Immunol. Res. 3(5):455-463 (2015). Monitoring completely treated HER2^{pos} s-BC patients for real-time fluctuations in anti-HER2 Th1 immunity, therefore, may reveal vulnerable populations at risk of clinicopathologic failure. Such immune monitoring may not only complement existing radiographic surveillance, but can also identify critical opportunities for therapeutic intervention, such as anti-HER2 Th1-directed immune interventions. See, Datta, et al. Breast Cancer Res.

Since immune responses were examined at the time of disease recurrence, it remains unclear whether anti-HER2 Th1 immunity in these patients remained suppressed following completion of index trastuzumab therapy or declined contemporaneously with the development of recurrence. Longitudinal surveillance of anti-HER2 Th1 responses prior to and following completion of HER2-targeted regimens in the context of future clinical trials appears warranted. Other limitations deserve to be mentioned. First, due to the nature of the study design for this Experimental Example, the findings herein should be interpreted as hypothesis-generating and warrant large-scale validation. Second, this study cannot address anti-HER2 Th1 immune repercussions following treatment with alternative HER2-targeted agents (e.g., lapatinib, pertuzumab, etc.). Finally, while the case for anti-HER2 Th1 immune monitoring in these patients is compelling, this study is unable to establish numerical thresholds for such monitoring. One skilled in the art can appreciate however that further studies will lead to the establishment of numerical thresholds that medical practitioners will be able to follow to monitor with blood tests as described herein and perhaps treat patients with anti-HER2-boosting therapies, such as the dendritic cell-based anti-HER2 vaccines described herein to restore anti-HER2 responsivity.

In sum, depressed anti-HER2 CD4⁺ Th1 response is a novel immune correlate to recurrence in HER2^{pos}-IBC patients following completion of HER2-targeted therapy. The data in this Experimental Example underscore a role for immune monitoring in completely treated HER2^{pos}-IBC patients to complement existing radiographic surveillance and identify vulnerable populations at risk of clinicopathologic failure.

In overall summary, circulating anti-HER2 Th1 immunity has been shown herein to be a dynamic correlate to relapse following trastuzumab therapy, with promising implications for immune monitoring and therapy selection in completely treated HER2^{pos}-BC patients. HER2-positive breast cancer patients will be able to have their immune status monitored with blood tests before, during, and after treatment, to allow physicians and other medical practitioners to gauge the risk of recurrence, and to reduce the risk of recurrence with therapies that boost anti-HER2 immunity such as, for example, DC vaccines or other cancer vaccines.

### Experimental Example 3

Anti-HER2 Th1 Response is Superior to Breast MRI in Assessing Response to Neoadjuvant Chemotherapy in Patients with HER2 Positive Breast Cancer

In HER2 positive breast cancer (“HER2⁺BC”) neoadjuvant chemotherapy (“nCT”) achieves complete pathologic response (“pCR”) ranging from 40-67%. Yuan Y, et al., Am. J. Radiol. 195(1):260-268 (2010). Post-treatment breast magnetic resonance imaging (pMRI) is currently considered the gold standard with high specificity (90.7%) but lower sensitivity (63.1%). Hylton, N. M., et al., Radiology 263(3):663-72 (2012 June). Anti-HER2 Th1 response is associated with pathologic response following nCT therapy in patients with HER2⁺BC. Datta, J., et al., Breast Cancer Res. 17:71. doi: 10.1186/s 13058-015-0584-1(2015 Nay 23) Identifying those patients with pCR can help tailor subsequent therapy. It critical to develop a sensitive tool to guide post neo-adjuvant treatment. In this study post treatment MRI was compared to anti-HER2 Th1 response for assessment of complete pathologic response in patients with HER2⁺BC.

### Methods

At the University of Pennsylvania 30 patients were retrospectively identified with histologically confirmed IBC and HER2/neu overexpression was confirmed. There was no evidence of distant metastasis in any of the patients and none were receiving immunosuppressive medications. Patients had anti-HER2 TH1 analysis performed in a non-biased fashion. Original post-treatment MRI reports were collected and imaging was reviewed by a breast radiologist who was blinded to pCR and immune response. Imaging-based tumor response was evaluated based on standard RECIST criteria, which was modified to include non-mass enhancement evaluated similar to solid lesions. Complete pathologic response was defined as an absence of residual invasive cancer on pathologic examination of resected breast specimen(s) and sampled lymph nodes (i.e., ypT0/Tis ypN0). As described in the Reference Example, circulating anti-HER2 CD4⁺ Th1 responses were examined in unstimulated PBMCs that were pulsed ex vivo with six HER2-derived MHC class II peptides (peptide 42-56, peptide 98-114, peptide 328-345, peptide 776-790, peptide 927-941, and peptide 1166-1180) (SEQ ID NOS: 1-6), by measuring IFN-γ production via enzyme-linked immunosorbent spot (ELISPOT) assays to determine cumulative responses. ELISPOT was performed as described in the Reference Example. MRI and anti-HER2Th1 responses were correlated with pathologic response and standard diagnostic metrics were computed.

The most commonly utilized treatment regimen was Adriamycin/Cyclophosphamide/Taxol/Herceptin. The most common surgical intervention was mastectomy. Patient characteristics are shown below:

| Patient Characteristics | |
|---|---|
| Age, years, mean ± standard error | 45.3 ± 2.1 |
| Age, years, range | 24-80 |
| BMI, mean ± standard error (kg/m2) | 2.3 ± 2.1 |
| Race/ethnicity, number (%) | |
| Caucasian | 35 (87.5) |
| African-American | 3 (7.5) |
| Asian | 1 (2.5) |
| Hispanic | 1 (2.5) |
| AJCC stage at diagnosis, number (%) | |
| Stage 1 | 0 (0) |
| Stage 2 | 21 (52.5) |
| Stage 3 | 19 (47.5) |
| Hormone receptor status, number (%) | |
| ER/PRpos | 18 (45.0) |
| ER/PRneg | 22 (55.0) |
| Lymphovascular Invasion Present, number (%) | 7 (17.5) |
| Time from completion of Trastuzumab to study | |
| enrollment, number (%) | |
| <6 months | 18 (45.0) |
| ≧6 months | 22 (55.0) |

### Results

The distribution of HER2 cumulative responses of the subjects by complete pathological responses (“PathCR”) was 13 patients had PathCR (Mean±SD=167.0±115.6 SFC/10⁶ cells, min=53.0 SFC/10⁶, max=418.0 SFC/10⁶ cells) and 17 patients did not have PathCR (Mean±SD=23.9±15.2 SFC/10⁶ cells, min=0.4 SFC/10⁶, max=53.3 SFC/10⁶ cells). Patients were dichotomized to a cutpoint of 50 SFC/10⁶ cells (“low” <50, “high” ≧50).

Diagnostic properties of MRI and HER2 cumulative response in HER2 patients are shown below. PPV=Positive Predictive Value; NPV=Negative Predictive Value.
1. MRI results versus Pathology results (gold standard), N=30 patients
   a. 13 had PathCR
   b. 17 no PathCR

| | No PathCR | PathCR | Total |
|---|---|---|---|
| MRI No | 11 | 8 | 19 |
| PathCR | | | |
| MRI PathCR | 6 | 5 | 11 |
| Total | 17 | 13 | 30 |

| | | |
|---|---|---|
| i. | Sensitivity | 5/13 = 38.5% |
| ii. | Specificity | 11/17 = 64.7% |
| iii. | PPV | 6/11 = 54.5% |
| iv. | NPV | 11/19 = 57.9% |
| v. | Overall accuracy | 16/30 = 53.3% |

2. ER negative, N=11 patients
   a. 8 had PathCR
   b. 3 no PathCR

| | No PathCR | PathCR | Total |
|---|---|---|---|
| MRI No | 2 | 6 | 8 |
| PathCR | | | |
| MRI PathCR | 1 | 2 | 3 |
| Total | 3 | 8 | 11 |

| | | |
|---|---|---|
| i. | Sensitivity | 2/8 = 25.0% |
| ii. | Specificity | 2/3 = 66.7% |
| iii. | PPV | 2/3 = 66.7% |
| iv. | NPV | 2/8 = 25.0% |
| v. | Overall accuracy | 4/11 = 36.3% |

3. ER positive, N=19 patients
   a. 5 had PathCR
   b. 14 no PathCR

| | No PathCR | PathCR | Total |
|---|---|---|---|
| MRI No | 9 | 2 | 11 |
| PathCR | | | |
| MRI PathCR | 5 | 3 | 8 |
| Total | 14 | 5 | 19 |

| | | |
|---|---|---|
| i. | Sensitivity | 3/5 = 60.0% |
| ii. | Specificity | 9/14 = 64.3% |
| iii. | PPV | 3/8 = 37.5% |
| iv. | NPV | 9/11 = 81.8% |
| v. | Overall accuracy | 12/19 = 63.2% |

4. Binary HER2_cumulative response results versus Pathology results (gold standard), N=30 patients
   a. 13 had PathCR
   b. 17 no PathCR
   c. 16 categorized as “low response” <50
   d. 14 categorized as “high response” ≧50

| | No PathCR | PathCR | Total |
|---|---|---|---|
| HER2 response | 16 | 0 | 16 |
| low | | | |
| HER2 response | 1 | 13 | 14 |
| high | | | |
| Total | 17 | 13 | 30 |

| | | |
|---|---|---|
| i. | Sensitivity | 13/13 = 100.0% |
| ii. | Specificity | 16/17 = 94.1% |
| iii. | PPV | 13/14 = 92.9% |
| iv. | NPV | 16/16 = 100.0% |
| v. | Overall accuracy | 29/30 = 96.7% |

5. ER negative, N=11 patients
   a. 8 had PathCR
   b. 3 no PathCR
   c. 3 categorized as “low response” <50
   d. 8 categorized as “high response” ≧50

| | No PathCR | PathCR | Total |
|---|---|---|---|
| HER2 response | 3 | 0 | 3 |
| low | | | |
| HER2 response | 0 | 8 | 8 |
| high | | | |
| Total | 3 | 8 | 11 |

| | | |
|---|---|---|
| i. | Sensitivity | 8/8 = 100.0% |
| ii. | Specificity | 3/3 = 100.0% |
| iii. | PPV | 8/8 = 100.0% |
| iv. | NPV | 3/3 = 100.0% |
| v. | Overall accuracy | 11/11 = 100.0% |

6. ER positive, N=19 patients
   a. 5 had PathCR
   b. 14 no PathCR
   c. 13 categorized as “low response” <50
   d. 6 categorized as “high response” ≧50

| | No PathCR | PathCR | Total |
|---|---|---|---|
| HER2 response | 13 | 0 | 13 |
| low | | | |
| HER2 response | 1 | 5 | 6 |
| high | | | |
| Total | 14 | 5 | 19 |

| | | |
|---|---|---|
| i. | Sensitivity | 5/5 = 100.0% |
| ii. | Specificity | 13/14 = 92.9% |
| iii. | PPV | 5/6 = 83.3% |
| iv. | NPV | 13/13 = 100.0% |
| v. | Overall accuracy | 18/19 = 94.7% |

7. Newly Reviewed MRI (“new MRI”) results versus Pathology results (gold standard), N=30 patients
   a. 13 had PathCR
   b. 17 no PathCR

| | No PathCR | PathCR | Total |
|---|---|---|---|
| New MRI No | 11 | 7 | 18 |
| PathCR | | | |
| New MRI | 6 | 6 | 12 |
| PathCR | | | |
| Total | 17 | 13 | 30 |

| | | |
|---|---|---|
| i. | Sensitivity | 6/13 = 46.2% |
| ii. | Specificity | 11/17 = 64.7% |
| iii. | PPV | 6/12 = 50.0% |
| iv. | NPV | 11/18 = 61.1% |
| v. | Overall accuracy | 17/30 = 56.7% |

8. ER negative, N=11 patients
   a. 8 had PathCR
   b. 3 no PathCR

| | No PathCR | PathCR | Total |
|---|---|---|---|
| New MRI No | 2 | 5 | 7 |
| PathCR | | | |
| New MRI | 1 | 3 | 4 |
| PathCR | | | |
| Total | 3 | 8 | 11 |

| | | |
|---|---|---|
| i. | Sensitivity | 3/8 = 37.5% |
| ii. | Specificity | 2/3 = 66.7% |
| iii. | PPV | 3/4 = 75.0% |
| iv. | NPV | 2/7 = 78.6% |
| v. | Overall accuracy | 5/11 = 45.5% |

9. ER positive, N=19 patients
   a. 5 had PathCR
   b. 14 no PathCR

| | No PathCR | PathCR | Total |
|---|---|---|---|
| New MRI eeNo | 9 | 2 | 11 |
| PathCR | | | |
| New MRI | 5 | 3 | 8 |
| PathCR | | | |
| Total | 14 | 5 | 19 |

| | | |
|---|---|---|
| i. | Sensitivity | 3/5 = 60.0% |
| ii | Specificity | 9/14 = 64.3% |
| iii. | PPV | 3/8 = 37.5% |
| iv. | NPV | 9/11 = 81.1% |
| v. | Overall accuracy | 12/19 = 63.1% |

The table below summarizes the data for all patients:

| | Sensitivity | Specificity | PPV | NPV | Accuracy |
|---|---|---|---|---|---|
| All Patients | | | | | |
| Original pMRI | 38.50% | 64.70% | 54.50% | 57.90% | 53.30% |
| report | | | | | |
| (Number 1. | | | | | |
| Data Above) | | | | | |
| Blinded re- | 46.20% | 64.70% | 50.00% | 61.10% | 56.70% |
| review pMRI | | | | | |
| (Number 8 Data | | | | | |
| Above) | | | | | |
| Anti-HER2 TH1 | 100.00% | 94.10% | 92.90% | 100.00% | 96.70% |
| Response | | | | | |
| (Number 5 Data | | | | | |
| Above) | | | | | |

Original pMRI had much lower diagnostic outcomes for pCR compared to anti-HER2 Th1 response as shown in the above table. In the subset of 28 patients with blinded review pMRI, pCR diagnostic outcomes remained noticeably inferior to anti-HER2 Th1 response (sensitivity 46.2% vs 100.0%, specificity 64.7% vs 94.1%, overall accuracy 56.7% vs 96.7%). Similar findings were observed when patients were stratified by estrogen receptor status, see, data for Numbers 2, 3, 4, 7, and 9 above.

Conclusion

Anti-HER2 Th1 immune response demonstrated strikingly accurate diagnostic metrics compared with post-treatment MRI. The presence of“high” anti-HER2 Th1 response is superior to the use of post-treatment MRI in the assessment of pCR in HER2⁺BC. Those having ordinary skill in the art will appreciate the advantages of monitoring patient immune responses in assessing response to neoadjuvant chemotherapy in HER2⁺BC patients using the methods presented herein.

### Experimental Example 4

### Improvements to the Dendritic Cell Activation Platform

Dendritic cells supply T cells with several signals that are required for their activation and inducement to fight the infection. The first signal (signal 1) is antigenic. The dendritic cells show, or “present” the microbial proteins to the T cell (this process is called antigen presentation). The second signal (signal 2) is costimulation. Dendritic cells produce their own transmembrane proteins called costimulatory molecules (including proteins named CD40, CD80 and DC86). These proteins interact with their counter-receptor on the T cell called CD28. Together, these signals activate the T lymphocyte, and are the minimal requirements to induce the T cell to go on a search-and-destroy mission against any target bearing the presented antigens (i.e. the invading microbe or potentially a cancerous cell). Dendritic cells, depending upon the precise circumstances of their own activation, can also conditionally produce a class of so-called “third signals” (signal 3). These tend to be members of a set of soluble immune system modifier peptides called cytokines. There are scores of known cytokines, and each possesses distinct functions. A subset of these cytokines can act as third signals to T cells during the process of antigen presentation. These 3^{rd} signals can be instrumental in influencing which functional capabilities will be acquired by the T cell to fight the infection. The dendritic cell essentially, upon contact with the invading microbe, makes a determination as to what type of T cell response will be best suited to eliminate the threat. The DC then produces the appropriate 3^{rd} signal cytokine that will inform the T cell which type of weapons it should bring to bear in the invader.

DCs sense infection in a number of ways. Their surfaces are studded with special transmembrane receptors called pattern recognition receptors. The best studied of these is a family called the Toll-like receptors (TLRs), of which there are 10 known members in humans. TLRs are triggered by biochemical structures common to broad classes of microbes that are correspondingly absent on normal healthy mammalian tissues. Each TLR is responsible for a different set of microbial structures. Some examples of these biochemical structures are lipopolysaccharide (LPS) and lipoteichoic acid (LTA), which are components of the cell walls of bacteria and are detected by the DC through TLR4 and TLR2, respectively. There are also synthetic compounds designed to trigger these receptors, such as R848, which is stimulatory to TLR8 and manufactured by 3M corporation. DCs are also capable of detecting evidence of infection through other means than TLRs. For example, the compound adenosine triphosphate (ATP) can be secreted by bacteria, or can be released by mammalian cells when they are damaged. DCs also possess purinergic receptors that can detect this extracellular ATP. Finally, DCs also possess receptors for a variety of cytokines. These cytokines can be produced by other immune system cells that have directly sensed infection, and their soluble nature allows the DC to be alerted indirectly, and at a distance. DCs must therefore be able to process large numbers of biochemical signals that provide them with information regarding the nature of the microbial threat they are facing. Many of these signals individually can induce DCs to enhance their capacity to produce signals 1 and 2 to T cells. However, they must integrate all of the many signals in the environment of an infection to make a decision as to which 3^{rd} signals to produce, since these will most profoundly affect T cell function, and the organism's ultimate ability to control and eliminate the infection.

T cells can develop into several different functional types, depending upon which third signals are present during their initial activation. There are 2 central types. The first is called a Th1 cell. Th1 T cells are identified by their tendency to produce very high amounts of the cytokine Interferon-gamma (IFN-γ). Th1 cells are adapted for dealing with microbes that have the ability to live inside certain cells of the body. They are also thought to be excellent for dealing with cancerous tumors. The dendritic cell produced 3^{rd} signal that induces T cells to acquire the properties of a Th1 is interleukin-12 (IL-12). A second type of T cell is the Th17. Th17 do not produce large amounts of IFN-γ but instead produce Interleukin-17 (IL-17) and Interleukin-22 (IL-22). These T cells appear adapted for dealing with certain infections that appear on mucous membranes. Th17 may play an anti-cancer role. The 3^{rd} signals that induce the Th17 phenotype are the DC-produced cytokines IL-23, IL-6, and IL-1β.

Because the correct “type” of T cells is so critical for fighting individual threats, and because dendritic cells help the T cell decide which type to become, immunologists interested in developing vaccines against infections and cancer have focused on dendritic cells, because by controlling the dendritic cell one can theoretically control key qualitative features (i.e., phenotype) of the T cell response. One issue is how to control which 3^{rd} signals DCs will produce, and thereby gain influence over T cell function in the developing immune response in order to produce maximal therapeutic DC vaccines, of which the herein embodiment of a pulsed HERs DC1 vaccine is an example.

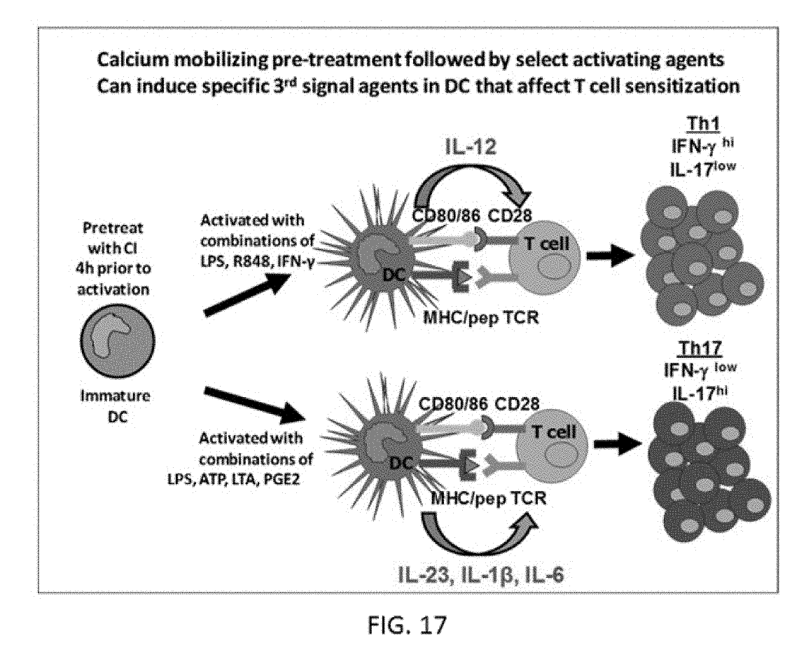

In a present embodiment, highly effective DCs are generated that produce strong, anti-tumor responses via calcium mobilizing co-treatment in combination with other DC activation regimens that enhance different and specific 3^{rd} signal agents in DC that affect T cell sensitization. Calcium ionophore paired with a combination of agents known to induce production of one or more of IL-23, IL-1β and IL-6 from DCs greatly enhances production of one or more of IL-23, IL-1β, and IL-6 and otherwise amplifies and improves other DC characteristics For example, FIG. 17 schematically shows pretreatment of immature DCs with calcium ionophore for 4 hours which causes calcium mobilization which pretreatment step is then paired with activation treatment combinations selected from TLR agonist LPS, adenosine triphosphate (ATP), bacterial lipoteichoic acid (LTA) and prostaglandin E2 (PGE2) to cause the third-signal agents IL-23, IL-6, and IL-1β to be amplified, leading an immune response dominated by IL-17 and IL-22-secreting Th17 cells in the virtual absence of IL-12. Without wishing to be bound by any particular theory, this combination drives development of the Th17 phenotype instead of Th1. Thus in a preferred embodiment DCs activated in this manner to induce specific 3^{rd} signal agents can then be pulsed with the HER2 MHC class II binding peptides and optionally with the HER2 MHC class I binding peptides (in the case the patient donor has a HLA A2.1 blood type) as described above.

In one exemplary embodiment, the DCs are suspended in human serum and approximately 10% DMSO (v/v). Alternatively, other serum types, such as human AB serum and fetal calf serum, may be used. The suspended cells can be aliquoted into smaller samples, such as in 1.8 ml vials, and stored at approximately −70° C. or lower. In other embodiments, the cryomedium may include about 20% serum and about 10% DMSO, and suspended cells can be stored at about −180° C. Still further embodiments may include medium containing about 55% oxypolygelatine, which is a plasma expander, about 6% hydroxyethylstarch, and about 5% DMSO. Other exemplary cryomediums may include about 12% DMSO and about 25-30% serum.

Similarly, while the present embodiments as described herein may include specific concentrations of DMSO, those skilled in the art should recognize that DMSO may be entirely absent in some embodiments, while in other embodiments, concentrations from about 5% to as high as about 20% may be used in the cryomedium and included within the cryopreservation methods described herein. Generally, lower concentrations of DMSO are preferred, such as between about 5% to about 10%. However, any concentration of DMSO that results, after thawing, in cell viability of at least 50% and a cell recovery of at least 50%, and preferably a cell viability and recovery of at least 60%, more preferably about 70%, more preferably about 80% and even more preferably about 90% and higher, may be used.

In one embodiment, the cryomedia comprises about 55% Plasma-lyte, about 40% human serum albumin, and about 5% DMSO.

In a particularly preferred embodiment the physical, chemical and pharmaceutical properties and formulation of the HER2-pulsed DC1 vaccine described in detail above are as follows. The product is preferably supplied as cryopreserved cell therapy. The product comprises autologous type I polarized DC pulsed with MHC class II peptides derived from HER-2 neu protein (SEQ ID NOS: 1-6) and optionally pulsed with MHC class I peptides derived from HER-2 neu protein in the case where donors have HLA A2.1 blood type (SEQ ID NOS: 7-8), or all 6 MHC class II and 2 MHS class I peptides may be pulsed onto the type I polarized DC. The product is cryopreserved in Plasma-Lyte, human AB serum, and contains 5% dimethyl sulfoxide (DMSO). Each vial contains between 10-20 million viable activated HER2-pulsed DC1 cells in 1 ml volume that are activated to secreted large quantities of Th1 cytokines such as, but not limited to IL-12, IL-6, and IL-23 and chemokines such as, but not limited to IP10 (CXCL10), MIG (CXCL 9), and RANTES (CCL5). The DC1 cells express varying amounts of costimulatory molecules, such as, but not limited to CD80, CD86, CCR7, CD83 on the cell surface. Cytokine and chemokine production have been shown to be maintained for up to 24-36-hours post-thaw.

In a randomized selection design trial, it was confirmed that neoadjuvant DC1 vaccination is a safe and immunogenic treatment in ductal carcinoma in situ (DCIS) and early invasive breast cancer (IBC), inducing a tumor-specific T-cell response in the peripheral blood and the sentinel lymph nodes independent of the route of vaccination (intralesional, intranodal, or both intralesional and intranodal). The pathologic complete response (pCR) rate was similar across all three injection routes, but was higher in patients with DCIS than in patients with stage I IBC. In DCIS patients, immune responses detected in the sentinel lymph nodes, but not peripheral blood, were associated with pCR. These findings suggest that (1) vaccines appear to be potentially more effective in DCIS and warrant further evaluation in DCIS or other minimal disease settings, and (2) the local regional sentinel lymph node may serve as a more meaningful immunologic endpoint.

The cryopreservation aspect of the embodiments allows for the generation of an FDA-approved injectable multi-dose antigen-pulsed dendritic cell vaccine. An advantage is that the multi-dose antigen-pulsed dendritic cells retain their ability to produce signals critical to T cell function after thawing. As contemplated herein, the present embodiments include a variety of cryopreservation techniques and cryomedia, as would be understood by those skilled in the art. Accordingly, the embodiments provide the ability to produce the multi-dose antigen-pulsed dendritic cell vaccine of the embodiments at a centralized area comprising an initial immunizing dose and multiple “booster” doses. Therefore, the multi-dose HER2-pulsed DC1 vaccine can be shipped to remote medical centers for serial administration to the patient with no special FDA quality control/quality assurance requirements at the administration site.

The disclosures of each and every patent, patent application, and publication cited herein are hereby incorporated herein by reference in their entirety.

This disclosure has been presented for purposes of illustration and description but is not intended to be exhausting or limiting. Many modifications and variations will be apparent to those of ordinary skill in the art. The embodiments were chosen and described in order to explain principles and practical application, and to enable others of ordinary skill in the art to understand the disclosure for various embodiments with various modifications as are suited to the particular use contemplated.

Although illustrative embodiments have been described herein with reference to the accompanying drawings, it is to be understood that the embodiments are not limited to those particular descriptions, and that various other changes and modifications may be devised therein by one skilled in the art without departing for the scope or spirit of the disclosure. The appended claims are intended to be construed to include all such embodiments and equivalent variations.

What is claimed is:

1. A method for diagnosing or treating a mammalian subject having, or at risk of developing cancer, comprising:
generating a circulating anti-cancer CD4⁺ Th1 response from antigen-presenting cells (“APCs”) or their precursors and CD4⁺ T-cells from a sample of said subject's blood which causes secretion of interferon-gamma (“IFN-γ”); and
detecting said anti-cancer CD4⁺ Th1 response to determine if said response is depressed.

2. The method of claim 1, wherein said generating step further comprises:
isolating unexpanded peripheral blood mononuclear cells (“PBMCs”) from said blood sample; and
pulsing said PBMCs and APC-precursor monocytes therein with a composition comprising immunogenic MHC class II binding peptides based on the type of cancer that afflicts said subject, thereby activating CD4⁺ Th1 cells in said PBMC's to secrete IFN-γ; and
said detection step comprises detecting said secreted IFN-γ.

3. The method of claim 1, wherein said generating step further comprises:
co-culturing purified CD4⁺ T-cells from said subject sample with APC immature or mature dendritic cells (“DCs”) from said subject sample pulsed with a composition comprising immunogenic MHC class II binding peptides based on the type of cancer that afflicts said subject, thereby activating said CD4⁺ T-cells to secrete IFN-γ; and
said detection step comprises detecting said secreted IFN-γ.

4. The method of claim 1, wherein said cancer is selected from the group consisting of breast, brain, bladder, esophagus, lung, pancreas, liver, prostate, ovarian, colorectal, and gastric cancer or any combination thereof.

5. The method of claim 4 wherein said cancer is HER2-expressing.

6. The method of claim 2, wherein said cancer is HER2-positive breast cancer, said subject is a human female, and said immunogenic MHC class II binding peptides are based on the HER2 molecule.

7. The method of claim 3, wherein said cancer is HER2-positive breast cancer, said subject is a human female, and said immunogenic MHC class II peptides are based on the HER2 molecule.

8. The method of claim 6 wherein said composition further comprises HER2 MHC class II binding peptides which comprise:

| | |
|---|---|
| Peptide 42-56: | |
| HLDMLRHLYQGCQVV; | (SEQ ID NO: 1) |
| Peptide 98-114: | |
| RLRIVRGTQLFEDNYAL; | (SEQ ID NO: 2) |
| Peptide 328-345: | |
| TQRCEKCSKPCARVCYGL; | (SEQ ID NO: 3) |
| Peptide 776-790: | |
| GVGSPYVSRLLGICL; | (SEQ ID NO: 4) |
| Peptide 927-941: | |
| PAREIPDLLEKGERL; | (SEQ ID NO: 5) |
| and | |
| Peptide 1166-1180: | |
| TLERPKTLSPGKNGV. | (SEQ ID NO: 6) |

9. The method of claim 7 wherein said composition further comprises HER2 MHC class II binding peptides which comprise:

| | |
|---|---|
| Peptide 42-56: | |
| HLDMLRHLYQGCQVV; | (SEQ ID NO: 1) |
| Peptide 98-114: | |
| RLRIVRGTQLFEDNYAL; | (SEQ ID NO: 2) |
| Peptide 328-345: | |
| TQRCEKCSKPCARVCYGL; | (SEQ ID NO: 3) |
| Peptide 776-790: | |
| GVGSPYVSRLLGICL; | (SEQ ID NO: 4) |
| Peptide 927-941: | |
| PAREIPDLLEKGERL; | (SEQ ID NO: 5) |
| and | |
| Peptide 1166-1180: | |
| TLERPKTLSPGKNGV. | (SEQ ID NO: 6) |

10. The method of claim 1 wherein said IFN-γ secretion is measured by IFN-γ enzyme-linked immunospot assay (“ELISPOT”).

11. A method for restoring HER2-specific CD4⁺ Th1 immune response in a HER2-positive breast cancer patient in need thereof, comprising:
administering to said patient a therapeutically effective amount of a DC vaccine comprising autologous DCs pulsed with immunogenic HER2 MHC class II binding peptides (“DC vaccination”) to elevate said patient's anti-HER2 CD4⁺ Th1 response; and
measuring said anti-HER2 CD4⁺Th1 response of said patient pre- and post-DC vaccination according to the method of claim 8 to determine the amount of increase in said response.

12. A method for restoring HER2-specific CD4⁺ Th1 immune response in a HER2-positive breast cancer patient in need thereof, comprising:
administering to said patient a therapeutically effective amount of a DC vaccine comprising autologous DCs pulsed with immunogenic HER2 MHC class II binding peptides (“DC vaccination”) to elevate said patient's anti-HER2 CD4⁺ Th1 response; and
measuring said anti-HER2 CD4⁺Th1 response of said patient pre- and post-DC vaccination according to the method of claim 9 to determine the amount of increase in said response.

13. The method of claim 11, further comprising:
measuring the status of said anti-HER2 CD4⁺Th1 response restoration of said patient post-DC vaccination by conducting the method of claim 8 at one or more additional time intervals to monitor said response restoration.

14. The method of claim 12, further comprising:
measuring the status of said anti-HER2 CD4⁺Th1 response restoration of said patient post-DC vaccination by conducting the method of claim 9 at one or more additional time intervals to monitor said response restoration.

15. A method for screening individuals for breast or other cancer, comprising:
detecting anti-HER2 CD4⁺ Th1 responses of said individuals according to the method of claim 1 to determine if said responses are depressed as compared to healthy individuals.

16. A method for screening individuals at risk for developing breast or other cancer, comprising:
detecting anti-HER2 CD4⁺ Th1 responses of said individuals according to the method of claim 1 to determine if said responses are depressed as compared to healthy individuals.

17. A method for predicting whether a patient with HER-positive breast cancer will respond well to standard non-immune therapy such as chemotherapy and trastuzumab, comprising:
detecting the anti-HER2 CD4⁺Th1 response of said patient according to the method of claim 1.

18. A method of predicting new breast events in HER2-positive-invasive breast cancer (“HER2^{pos}-IBC”) patients treated with trastuzumab and chemotherapy, comprising:
measuring the anti-HER2 CD4⁺Th1 response of said patient according to the method of claim 1 to determine if said response is depressed.

19. A method of predicting pathologic response of HER2-positive breast cancer following neoadjuvant trastuzumab and chemotherapy (“T/C”) therapy in a HER2-positive breast cancer patient, comprising:
measuring the degree of anti-HER2 CD4⁺ Th1 responsiveness in said patient post-T/C treatment according to the method of claim 1 to determine if said response is a significantly higher anti-HER2 CD4⁺ Th1 response associated with neoadjuvant pathological complete response (no residual invasive breast cancer on postoperative pathology) or a lower response associated with non-pathological complete response.

20. The method of claim 19, wherein in the case of a non-pathological complete response in said patient, the anti-HER2 CD4⁺ Th1 response of said patient is restored by DC vaccination according to the method of claim 11.

21. A method for diagnosing or treating a mammalian subject having, or at risk of developing cancer, comprising:
obtaining blood from said subject;
performing a blood test thereon which measures suppression in anti-cancer CD4+ Th1 response, and in the case of suppression;
administering to said subject a cancer medicament in an effective amount selected from the group consisting of DC vaccine, targeted cancer therapy such as trastuzumab, conventional cancer therapy such as chemotherapy, surgery, and radiation.

22. A method of monitoring a HER2^{pos}-IBC patient following completion of a targeted breast cancer therapy plus chemotherapy to assess the risk of recurrence of said cancer, comprising: measuring the degree of anti-HER2 CD4⁺ Th1 responsiveness in said patient post-therapy according to the method of claim 1 to determine if said response is a significantly depressed anti-HER2 CD4⁺ Th1 response that correlates with recurrence of said cancer or a higher anti-HER2 CD4⁺ Th1 response that correlates with non-recurrence.

23. The method of claim 22, wherein said targeted therapy comprises administration of the drug trastuzumab to said patient.

24. An immune therapy for restoration of the pre-existing component of a subject's immunity that is lost upon the development of cancer in said subject.

25. The immune therapy of claim 24, wherein said pre-existing component of immunity of said subject is anti-HER2 TH1 immune response.

26. The immune therapy of claim 25, wherein said subject has HER2^{pos} DCIS or HER2^{pos} IBC breast cancer.

27. The immune therapy of claim 26, wherein said subject's anti-HER2 TH1 immune response is restored via administration of HER2-pulsed DC1 vaccine.
